(19) United States
(12) Patent Application Publication
(10) Pub. No.: US 2009/0221829 A1
(43) Pub. Date: Sep. 3, 2009

(54) 3,4-Dihydroisoquinolinium Salt Derivatives

(76) Inventors: Jungho Kim, Daejeon (KR); Sangphil Lee, Daejeon (KR); Junyoung Choi, Daejeon (KR); Young-Ki Paik, Seoul (KR); Yousuk Lee, Seoul (KR); Hyungmin Joo, Daejeon (KR)

Correspondence Address: THE NATH LAW GROUP, 112 South West Street, Alexandria, VA, 22314, US

(21) Appl. No.: 11/921,041

(22) PCT Filed: Jun. 1, 2006
(86) PCT No.: PCT/KR2006/002113

§ 371 Date: Feb. 13, 2009

(30) Foreign Application Priority Data
Jun. 1, 2005 (KR) 10-2005-0046749

(51) Int. Cl. C07D 217/04; C07D 217/00

(52) U.S. Cl. 546/146; 546/149

(57) ABSTRACT

The present invention relates to 3,4-dihydroisoquinolinium salt derivatives. More specifically, the present invention relates to 3,4-dihydroisoquinolinium salt derivatives of the following chemical formula (I).

### TECHNICAL FIELD

The present invention relates to 3,4-dihydroisoquinolinium salt derivatives. More specifically, the present invention is directed to 3,4-dihydroisoquinolinium salt derivatives of the following chemical formula

(I):

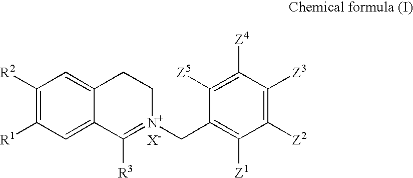

wherein R¹ and R² which may be the same or different from each other, represent a hydrogen, halogen or alkoxy group or R¹ and R² together represent a methylenedioxy group, C₁-C₂ alkoxycarbonylamino or C₁-C₃ alkylamino group;

R³ represents a hydrogen, alkyl group, C₁-C₁ alkenyl group, phenyl, substituted phenyl, benzyl or arylalkyl group;

Z¹, Z², Z³, Z⁴ and Z⁵ which may be the same or different from each other, represent a hydrogen, halogen, C₁-C₅ alkyl group, trifluoromethyl, phenyl, substituted phenyl, nitro, C₁-C₄ alkoxy group, trifluoromethoxy, hydroxy, phenoxy, substituted benzyloxy, methoxycarboxyl group, C₁-C₄ alkoxycarbonyl group or ammonium group;

X⁻ represents inorganic acid ion, organic acid ion or a halide.

### BACKGROUND ART

Fungal infection may be classified into dermatomycosis and systemic mycosis. Researches have been focused on the development of new type of antifungal agents for systemic mycosis since the systemic mycosis may cause a fatal effect on human body. Particularly, some pathogenic fungal germs, such as Aspergillus and Candida which may occur under the specific conditions of immune deficiency, are substantial cause of human death. That is, patients who have immune deficiency such as a person with AIDS, lead to death by fungal infection on tissue or blood.

In order to inhibit inhabitation and growth of fungus in human body, it is very important to control lipid metabolism of fungus. The Ergosterol which is a typical lipid in fungal cell, is a vital constituent of cell membrane and has great effect on cell division, growth and metabolic movement.

Antifungal drug has also been studied for a long time upon focusing on inhibiting the synthesis of sterol since the growth of fungus depends on biosynthesis of sterol.

Polyene and azole compounds have been known and used generally as an antifungal drug.

The azole antifungal drug controls fungus by inhibiting sterol 14-α demethylase which is required for a process for biosynthesis of sterol of a mold. However, the azole antifungal may cause side effects such as hepatotoxicity and nephrotoxicity since it also inhibits sterol 14-α demethylase which exists in human body.

The polyene antifungal drug such as Amphotericin B which inhibits a process for biosynthesis of ergosterol of fungus also has a difficulty for using clinically since they may cause side effects such as severe rigor, myalgia and nephrotoxicity on human.

Accordingly, it has been required to develop an effective antifungal which has less side effects, hardly develops resistance even though it is administered to a patient for a long period.

### DISCLOSURE OF INVENTION

### Technical Problem

It is an primary object of the present invention to provide 3,4-dihydroisoquinolinium salt derivatives of the following chemical formula (I):

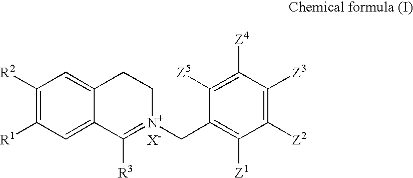

wherein R¹ and R² which may be the same or different from each other, represent a hydrogen, halogen or alkoxy group or R¹ and R² together represent a methylenedioxy group, C₁-C₂ alkoxycarbonylamino or C₁-C₃ alkylamino group;

R³ represents a hydrogen, alkyl group, C₁-C₁₈ alkenyl group, phenyl, substituted phenyl, benzyl or arylalkyl group;

Z¹, Z², Z³, Z⁴ and Z⁵ which may be the same or different from each other, represent a hydrogen, halogen, C₁-C₅ alkyl group, trifluoromethyl, phenyl, substituted phenyl, nitro, C₁-C₄ alkoxy group, trifluoromethoxy, hydroxy, phenoxy, substituted benzyloxy, methoxycarboxyl group, C₁-C₄ alkoxycarbonyl group or ammonium group;

X⁻ represents inorganic acid ion, organic acid ion or a halide.

Another object of the present invention is to provide isoquinolinium salt derivatives of the following chemical formula (II).

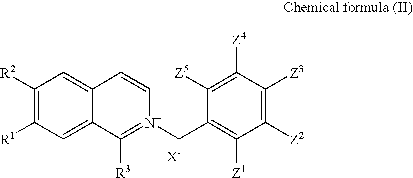

In the above chemical formula (II), R¹ and R² which may be the same or different from each other, represent a hydrogen, halogen or alkoxy group or R¹ and R² together represent methylenedioxy group, C₁-C₂ alkoxycarbonylamino or C₁-C₃ alkylamino group;

R³ represents a hydrogen, alkyl group, C₁-C₁₈ alkenyl group, phenyl, substituted phenyl, benzyl or arylalkyl group;

Z¹, Z², Z³, Z⁴ and Z⁵ which may be the same or different from each other, represent a hydrogen, halogen, C₁-C₅ alkyl group, trifluoromethyl, phenyl, substituted phenyl, nitro, C₁-C₄ alkoxy, trifluoromethoxy, hydroxy, phenoxy, substituted benzyloxy, methoxycarboxyl group, C₁-C₄ alkoxycarbonyl group or ammonium group;

X⁻ represents inorganic acid ion, organic acid ion or a halide.

Yet another object of the present invention is to provide a pharmaceutical composition containing pharmaceutically effective amount of 3,4-dihydroisoquinolinium salt derivatives of the chemical formula (I).

A still another object of the present invention is to provide pharmaceutical composition containing pharmaceutically effective amount of isoquinolinium salt derivatives of the chemical formula (If).

A further another object of the present invention is to provide a process for preparing 3,4-dihydroisoquinolinium salt derivatives which comprises 3) a step for preparing the following chemical formula (VI) by reacting a compound of the following chemical formula (VI) with the following chemical formula (IV); □) a step for preparing the following chemical formula (VII) by reacting a compound of the chemical formula (VI) which is obtained from the above step □) with acyl halide; and □) a step for reacting a compound of the chemical formula (VII) which is obtained from the above step □) in the presence of a catalyst:

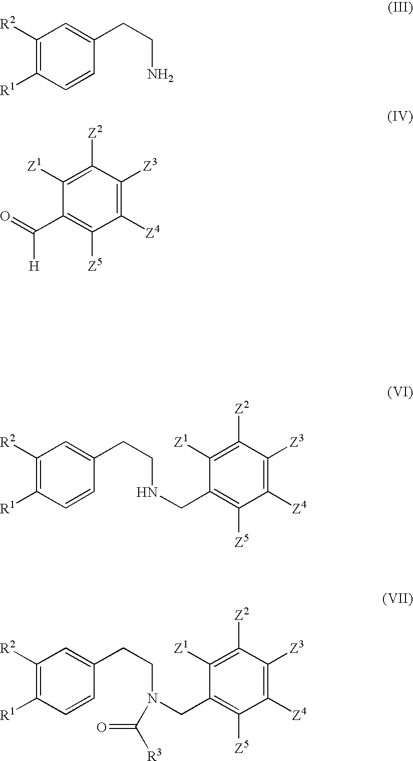

wherein, R¹, R², R³, Z¹, Z², Z³, Z⁴, Z⁵ and X⁻ are as defined above.

A still another object of the present invention is to provide a process for preparing 3,4-dihydroisoquinolinium salt derivatives which comprises □) a step for preparing the following chemical formula (VI) by reacting a compound of the following chemical formula (III) with a compound of the following chemical formula (V); □) a step for preparing the following chemical formula (VII) by reacting a compound of the chemical formula (VI) which is obtained from the above step □) with acyl halide; and □) a step for reacting a compound of the chemical formula (VII) which is obtained from the above step □) in the presence of a catalyst:

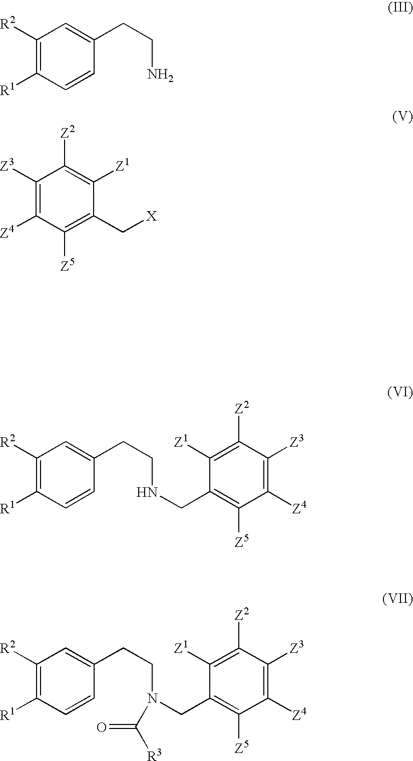

wherein, R¹, R², R³, Z¹, Z², Z³, Z⁴, Z⁵ and X⁻ are as defined above.

A further another object of the present invention is to provide a process for preparing 3,4-dihydroisoquinolinium salt derivatives which comprises □) a step for preparing a compound of the following chemical formula (□) by reacting a compound of the following chemical formula (III) with acyl halide; □) a step for preparing a compound of the following chemical formula (IX) by reacting a compound of the chemical formula (□) which is obtained from the above step □) in the presence of a catalyst; □) a step for reacting a compound of the chemical formula (IX) which is obtained from the above step □) with a compound of the following chemical formula (V):

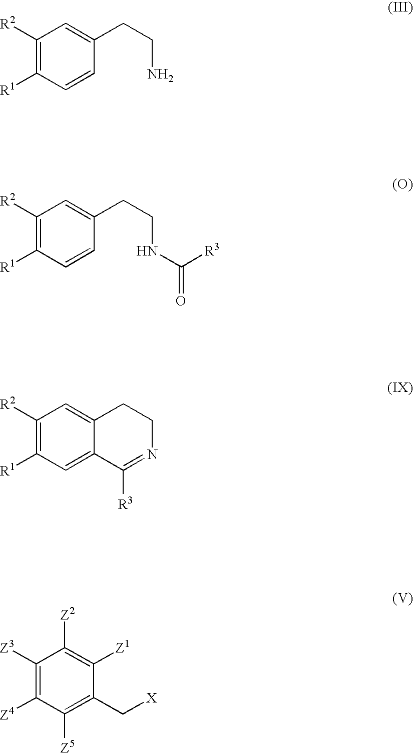

wherein, R¹, R², R³, Z¹, Z², Z³, Z⁴, Z⁵ and X⁻ are as defined above.

A still another object of the present invention is to provide a process for preparing isoquinolinium salt derivatives by reacting a compound of the following chemical formula (X) with a compound of the following chemical formula (V):

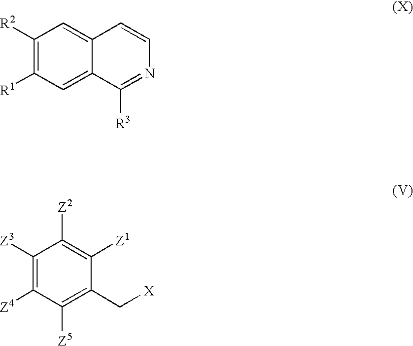

wherein, R¹, R², R³, Z¹, Z², Z³, Z⁴, Z⁵ and X⁻ are as defined above.

### Technical Solution

The above objects of the present invention may be achieved by providing the 3,4-dihydroisoquinlolinium salt derivatives of the following chemical formula (I).

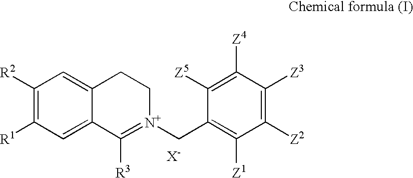

In the above chemical formula (I), R¹ and R² which may be the same or different from each other, represent a hydrogen, halogen or alkoxy group or R¹ and R² together represent a methylenedioxy group, C₁-C₂ alkoxycarbonylamino or C₁-C₃ alkylamino group;

R³ represents a hydrogen, alkyl group, C₁-C₁₈ alkenyl group, phenyl, substituted phenyl, benzyl or arylalkyl group;

Z¹, Z², Z³, Z⁴ and Z⁵ which may be the same or different from each other, represent a hydrogen, halogen, C₁-C₅ alkyl group, trifluoromethyl, phenyl, substituted phenyl, nitro, C₁-C₄ alkoxy group, trifluoromethoxy, hydroxy, phenoxy, substituted benzyloxy, methoxycarboxyl group, C₁-C₄ alkoxycarbonyl group or ammonium group;

X⁻ represents inorganic acid ion, organic acid ion or a halide.

Another object of the present invention may be achieved by providing iso-quinolinium salt derivatives of the following chemical formula (II).

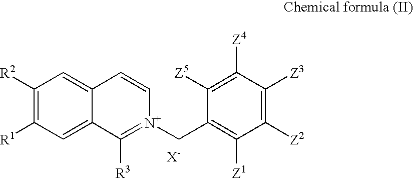

In the above chemical formula (II), R¹ and R² which may be the same or different from each other, represent a hydrogen, halogen or alkoxy group or R¹ and R² together represent a methylenedioxy group, C₁-C₂ alkoxycarbonylamino or C₁-C₃ alkylamino group;

R³ represents a hydrogen, alkyl group, C₁-C₁₈ alkenyl group, phenyl, substituted phenyl, benzyl or arylalkyl group;

Z¹, Z², Z³, Z⁴ and Z⁵ which may be the same or different from each other, represent a hydrogen, halogen, C₁-C₅ alkyl group, trifluoromethyl, phenyl, substituted phenyl, nitro, C₁-C₄ alkoxy group, trifluoromethoxy, hydroxy, phenoxy, substituted benzyloxy, methoxycarboxyl group, C₁-C₄ alkoxycarbonyl group or ammonium group;

X⁻ represents inorganic acid ion, organic acid ion or a halide.

For example, the above 3,4-dihydroisoquinolinium salt derivatives and iso-quinolinium salt derivatives are represented by Table 1.

Novel compounds represented by the above chemical formula (I), and the relative activities in accordance with the agar dilution method in Sabouraud dextrose agar media, Czapek agar media and Yeast Extract-Peptone-Dextrose agar media for Candida albicans(KCTC 1940), Aspergillus niger (ATCC 9642) and Saccharomyces cerevisiae are described respectively in following Table 1.

The relative activities of novel compounds are evaluated and expressed as follows: the relative activity is 4 in case that the control drug, i.e., Miconazole exhibits the fungistatic activity in agar media at a certain concentration; the relative activity of the novel compound is 4 in case that the novel compound exhibit the fungicidal activity at the concentration same as that of Miconazole; the relative activities of the novel compound are 3, 2, 1 respectively in case that the novel compound exhibits the fungicidal activity at 2, 4, 8 times higher concentration than that of Miconazole; the relative activities of the novel compound are 5, 6, 7 respectively in case that the novel compound exhibits the fungicidal activity at ½, ¼, ⅛ times lower concentration than that of Miconazole.

TABLE 1

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 1 | CH₃O | CH₃O | H | Cl | F | H | H | H | H | 6 |
| 2 | CH₃O | CH₃O | CH₃ | Cl | F | H | H | H | H | 6 |
| 3 | CH₃O | CH₃O | CH₂Cl | Cl | F | H | H | H | H | 6 |
| 4 | CH₃O | CH₃O | Et | Cl | F | H | H | H | H | 6 |
| 5 | CH₃O | CH₃O | n-Pr | Cl | F | H | H | H | H | 5 |
| 6 | CH₃O | CH₃O | i-Pr | Cl | F | H | H | H | H | 5 |
| 7 | CH₃O | CH₃O | (CH₂)₃Cl | Cl | F | H | H | H | H | 5 |
| 8 | CH₃O | CH₃O | CH₂CH(CH₃)₂ | Cl | F | H | H | H | H | 5 |
| 9 | CH₃O | CH₃O | n-Pentyl | Cl | F | H | H | H | H | 4 |
| 10 | CH₃O | CH₃O | n-Hexyl | Cl | F | H | H | H | H | 4 |
| 11 | CH₃O | CH₃O | n-Heptyl | Cl | F | H | H | H | H | 4 |
| 12 | CH₃O | CH₃O | (CH₂)₁₀CH₃ | Cl | F | H | H | H | H | 2 |
| 13 | CH₃O | CH₃O | (CH₂)₁₄CH₃ | Cl | F | H | H | H | H | 2 |
| 14 | CH₃O | CH₃O | 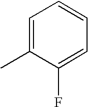 | Cl | F | H | H | H | H | 6 |
| 15 | CH₃O | CH₃O | 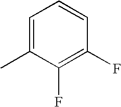 | Cl | F | H | H | H | H | 6 |

TABLE 2

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 16 | CH₃O | CH₃O | 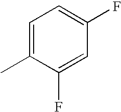 | Cl | F | H | H | H | H | 6 |
| 17 | CH₃O | CH₃O | 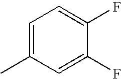 | Cl | F | H | H | H | H | 6 |
| 18 | CH₃O | CH₃O | 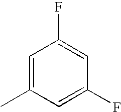 | Cl | F | H | H | H | H | 6 |
| 19 | CH₃O | CH₃O | 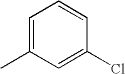 | Cl | F | H | H | H | H | 6 |
| 20 | CH₃O | CH₃O | 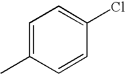 | Cl | F | H | H | H | H | 6 |
| 21 | CH₃O | CH₃O | 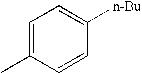 | Cl | F | H | H | H | H | 6 |
| 22 | CH₃O | CH₃O | 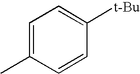 | Cl | F | H | H | H | H | 6 |
| 23 | CH₃O | CH₃O | 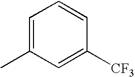 | Cl | F | H | H | H | H | 6 |
| 24 | CH₃O | CH₃O | 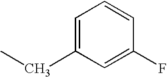 | Cl | F | H | H | H | H | 6 |
| 25 | CH₃O | CH₃O | 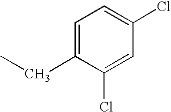 | Cl | F | H | H | H | H | 6 |
| 26 | CH₃O | CH₃O | CH₃ | Cl | H | H | CF₃ | H | H | 6 |
| 27 | CH₃O | CH₃O | n-Pentyl | Cl | H | H | CF₃ | H | H | 4 |
| 28 | CH₃O | CH₃O | n-Heptyl | Cl | H | H | CF₃ | H | H | 4 |
| 29 | CH₃O | CH₃O | (CH₂)₁₀CH₃ | Cl | H | H | CF₃ | H | H | 1 |
| 30 | CH₃O | CH₃O | (CH2)₁₄CH₃ | Cl | H | H | CF₃ | H | H | 1 |
| 31 | CH₃O | CH₃O | 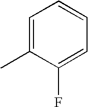 | Cl | H | H | CF₃ | H | H | 6 |

TABLE 3

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 32 | CH₃O | CH₃O | 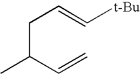 | Cl | H | H | CF₃ | H | H | 6 |
| 33 | CH₃O | CH₃O | CH₃ | Cl | H | H | OCH₃ | H | H | 6 |
| 34 | CH₃O | CH₃O | n-Pentyl | Cl | H | H | OCH₃ | H | H | 4 |
| 35 | CH₃O | CH₃O | n-Hexyl | Cl | H | H | OCH₃ | H | H | 4 |
| 36 | CH₃O | CH₃O | n-Heptyl | Cl | H | H | OCH₃ | H | H | 3 |
| 37 | CH₃O | CH₃O | (CH₂)₁₀CH₃ | Cl | H | H | OCH₃ | H | H | 2 |
| 38 | CH₃O | CH₃O | (CH₂)₁₄CH₃ | Cl | H | H | OCH₃ | H | H | 1 |
| 39 | CH₃O | CH₃O | CH₃ | Cl | H | F | F | H | H | 6 |
| 40 | CH₃O | CH₃O | i-Pr | Cl | H | F | F | H | H | 6 |
| 41 | CH₃O | CH₃O | n-Pentyl | Cl | H | F | F | H | H | 6 |
| 42 | CH₃O | CH₃O | n-Hexyl | Cl | H | F | F | H | H | 4 |
| 43 | CH₃O | CH₃O | n-Heptyl | Cl | H | F | F | H | H | 3 |
| 44 | CH₃O | CH₃O | (CH₂)₁₀CH₃ | Cl | H | F | F | H | H | 1 |
| 45 | CH₃O | CH₃O | n-Hexyl | Cl | H | H | CF₃ | H | H | 4 |
| 46 | CH₃O | CH₃O | (CH₂)₁₄CH₃ | Cl | H | F | F | H | H | 1 |

TABLE 4

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 47 | CH₃O | CH₃O | 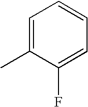 | Cl | H | F | F | H | H | 6 |
| 48 | CH₃O | CH₃O | 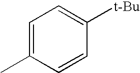 | Cl | H | F | F | H | H | 6 |
| 49 | CH₃O | CH₃O | CH₃ | Cl | Cl | H | H | H | H | 6 |
| 50 | CH₃O | CH₃O | n-Pentyl | Cl | Cl | H | H | H | H | 6 |
| 51 | CH₃O | CH₃O | n-Hexyl | Cl | Cl | H | H | H | H | 4 |
| 52 | CH₃O | CH₃O | n-Heptyl | Cl | Cl | H | H | H | H | 3 |
| 53 | CH₃O | CH₃O | (CH₂)₁₀CH₃ | Cl | Cl | H | H | H | H | 2 |
| 54 | CH₃O | CH₃O | (CH₂)₁₄CH₃ | Cl | Cl | H | H | H | H | 1 |
| 55 | CH₃O | CH₃O | 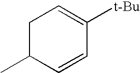 | Cl | Cl | H | H | H | H | 6 |
| 56 | CH₃O | CH₃O | CH₃ | Cl | F | H | H | H | F | 6 |
| 57 | CH₃O | CH₃O | n-Pentyl | Cl | F | H | H | H | F | 6 |
| 58 | CH₃O | CH₃O | n-Hexyl | Cl | F | H | H | H | F | 4 |
| 59 | CH₃O | CH₃O | (CH₂)₁₀CH₃ | Cl | F | H | H | H | F | 1 |
| 60 | CH₃O | CH₃O | (CH₂)₁₄CH₃ | Cl | F | H | H | H | F | 1 |

TABLE 5

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 61 | CH₃O | CH₃O | CH₃ | Cl | F | H | H | H | F | 4 |
| 62 | CH₃O | CH₃O | 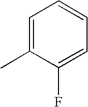 | Cl | F | H | H | H | F | 6 |
| 63 | CH₃O | CH₃O | 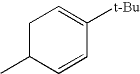 | Cl | F | H | H | H | F | 6 |
| 64 | CH₃O | CH₃O | CH₃ | Cl | Cl | H | H | H | F | 6 |
| 65 | CH₃O | CH₃O | i-Pr | Cl | Cl | H | H | H | F | 5 |
| 66 | CH₃O | CH₃O | n-Pentyl | Cl | Cl | H | H | H | F | 5 |
| 67 | CH₃O | CH₃O | n-Heptyl | Cl | Cl | H | H | H | F | 4 |
| 68 | CH₃O | CH₃O | (CH₂)₁₀CH₃ | Cl | Cl | H | H | H | F | 2 |
| 69 | CH₃O | CH₃O | 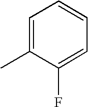 | Cl | Cl | H | H | H | F | 6 |
| 70 | CH₃O | CH₃O | 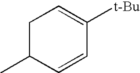 | Cl | Cl | H | H | H | F | 6 |
| 71 | CH₃O | CH₃O | CH₃ | Cl | NO₂ | H | H | H | H | 6 |
| 72 | CH₃O | CH₃O | i-Pr | Cl | NO₂ | H | H | H | H | 6 |
| 73 | CH₃O | CH₃O | n-Heptyl | Cl | NO₂ | H | H | H | H | 5 |
| 74 | CH₃O | CH₃O | (CH₂)₁₀CH₃ | Cl | NO₂ | H | H | H | H | 2 |
| 75 | CH₃O | CH₃O | 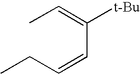 | Cl | NO₂ | H | H | H | H | 6 |

TABLE 6

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 76 | CH₃O | CH₃O | n-Heptyl | Cl | H | NO₂ | OH | H | H | 4 |
| 77 | CH₃O | CH₃O | (CH₂)₁₀CH₃ | Cl | H | NO₂ | OH | H | H | 2 |
| 78 | CH₃O | CH₃O | CH₃ | Cl | H | Br | OCH₃ | OCH₃ | H | 6 |
| 79 | CH₃O | CH₃O | n-Heptyl | Cl | H | Br | OCH₃ | OCH₃ | H | 5 |
| 80 | H | CH₃O | CH₃ | Cl | F | H | H | H | H | 6 |
| 81 | H | CH₃O | Et | Cl | F | H | H | H | H | 6 |
| 82 | H | CH₃O | n-Pr | Cl | F | H | H | H | H | 6 |
| 83 | H | CH₃O | i-Pr | Cl | F | H | H | H | H | 6 |
| 84 | H | CH₃O | CH₂CH(CH₃)₂ | Cl | F | H | H | H | H | 6 |
| 85 | H | CH₃O | n-Pentyl | Cl | F | H | H | H | H | 5 |
| 86 | H | CH₃O | n-Hexyl | Cl | F | H | H | H | H | 5 |
| 87 | H | CH₃O | n-Heptyl | Cl | F | H | H | H | H | 5 |
| 88 | H | CH₃O | (CH₂)₁₀CH₃ | Cl | F | H | H | H | H | 2 |
| 89 | H | CH₃O | (CH₂)₁₄CH₃ | Cl | F | H | H | H | H | 1 |
| 90 | H | CH₃O | 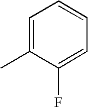 | Cl | F | H | H | H | H | 6 |
| 91 | H | CH₃O | 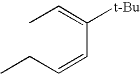 | Cl | F | H | H | H | H | 6 |

TABLE 7

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 76 | CH₃O | CH₃O | n-Heptyl | Cl | H | NO₂ | OH | H | H | 4 |
| 77 | CH₃O | CH₃O | (CH₂)₁₀CH₃ | Cl | H | NO₂ | OH | H | H | 2 |
| 78 | CH₃O | CH₃O | CH₃ | Cl | H | Br | OCH₃ | OCH₃ | H | 6 |
| 79 | CH₃O | CH₃O | n-Heptyl | Cl | H | Br | OCH₃ | OCH₃ | H | 5 |
| 80 | H | CH₃O | CH₃ | Cl | F | H | H | H | H | 6 |
| 81 | H | CH₃O | Et | Cl | F | H | H | H | H | 6 |
| 82 | H | CH₃O | n-Pr | Cl | F | H | H | H | H | 6 |
| 83 | H | CH₃O | i-Pr | Cl | F | H | H | H | H | 6 |
| 84 | H | CH₃O | CH₂CH(CH₃)₂ | Cl | F | H | H | H | H | 6 |
| 85 | H | CH₃O | n-Pentyl | Cl | F | H | H | H | H | 5 |
| 86 | H | CH₃O | n-Hexyl | Cl | F | H | H | H | H | 5 |
| 87 | H | CH₃O | n-Heptyl | Cl | F | H | H | H | H | 5 |
| 88 | H | CH₃O | (CH₂)₁₀CH₃ | Cl | F | H | H | H | H | 2 |
| 89 | H | CH₃O | (CH₂)₁₄CH₃ | Cl | F | H | H | H | H | 1 |
| 90 | H | CH₃O | 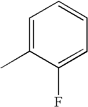 | Cl | F | H | H | H | H | 6 |
| 91 | H | CH₃O | 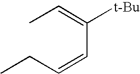 | Cl | F | H | H | H | H | 6 |

TABLE 8

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 107 | —OCH₂O— | | CH₃ | Cl | H | H | CF₃ | H | H | 6 |
| 108 | —OCH₂O— | | (CH₂)₆CH₃ | Cl | H | H | CF₃ | H | H | 4 |
| 109 | —OCH₂O— | | (CH₂)₁₄CH₃ | Cl | H | H | CF₃ | H | H | 1 |
| 110 | —OCH₂O— | | 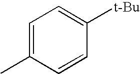 | Cl | H | H | CF₃ | H | H | 6 |
| 111 | —OCH₂O— | | CH₃ | Cl | H | H | F | F | H | 6 |
| 112 | —OCH₂O— | | (CH₂)₆CH₃ | Cl | H | H | F | F | H | 4 |
| 113 | —OCH₂O— | | (CH₂)₁₄CH₃ | Cl | H | H | F | F | H | 1 |
| 114 | —OCH₂O— | | 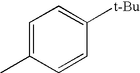 | Cl | H | H | F | F | H | 6 |
| 115 | CH₃OCONH | | (CH₂)₁₀CH₃ | Br | F | H | t-Bu | H | H | 2 |
| 116 | EtOCO | CH₃O | (CH₂)₁₀CH₃ | Br | H | H | t-Bu | H | H | 2 |
| | NCH₃ | | | | | | | | | |
| 117 | EtOCO | H | (CH₂)₁₀CH₃ | Br | H | H | CF₃ | H | H | 4 |
| | NCH₃ | | | | | | | | | |
| 118 | EtOCO | H | (CH₂)₁₀CH₃ | Br | H | H | H | t-Bu | H | 2 |
| | NCH₃ | | | | | | | | | |
| 119 | CH₃NH | CH₃O | (CH₂)₁₀CH₃ | Br | H | H | H | t-Bu | H | 1 |

| | | | | | | | | | |
|---|---|---|---|---|---|---|---|---|---|
| 120 | CH₃O | CH₃O | (CH₂)₁₀CH₃ | Cl | H | H | H | t-BuOCO | H |
| | | | | | | | | NH | |

TABLE 9

| | | | | | | | | | | Rela- |
|---|---|---|---|---|---|---|---|---|---|---|
| | | | | | | | | | | tive |
| Com- | | | | | | | | | | Ac- |
| pound | | | | | | | | | | tiv- |
| No. | R¹ | R² | R³ | X | Z¹ | Z² | Z³ | Z⁴ | Z⁵ | ity |
| 121 | CH₃O | CH₃O | (CH₂)₁₀CH₃ | Cl | H | H | H | NH₃ + | H | 2 |
| | | | | | | | | Cl— | | |
| 122 | CH₃O | CH₃O | (CH₂)₁₀CH₃ | Cl | H | H | NHCO₂Et | NHCO₂Et | H | 2 |
| 123 | CH₃O | CH₃O | n-Pr | Cl | H | H | t-Bu | H | H | 6 |
| 124 | CH₃O | CH₃O | 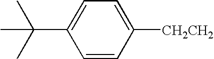 | Cl | F | H | H | H | H | 2 |
| 125 | CH₃O | CH₃O | 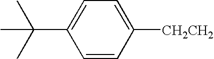 | Cl | H | H | OMe | OMe | H | 2 |
| 126 | CH₃O | CH₃O | (CH₂)₅CH₃ | Cl | H | H | t-Bu | H | H | 4 |
| 127 | CH₃O | CH₃O | (CH₂)₁₀CH₃ | Cl | H | H | t-Bu | H | H | 1 |
| 128 | CH₃O | CH₃O | (CH₂)₁₄CH₃ | Cl | H | H | t-Bu | H | H | 1 |
| 129 | CH₃O | CH₃O | 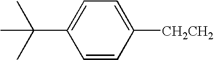 | Br | H | H | CF₃ | H | H | 4 |
| 130 | CH₃O | CH₃O | 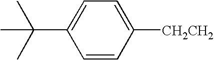 | Cl | F | F | F | F | F | 4 |
| 131 | CH₃O | CH₃O | 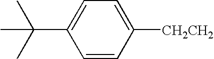 | Cl | F | F | CF₃ | F | F | 4 |
| 132 | CH₃O | CH₃O | 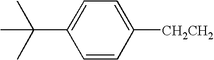 | Cl | 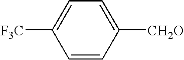 | OMe | H | H | H | 1 |
| 133 | CH₃O | CH₃O | 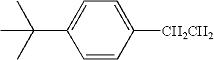 | Cl | 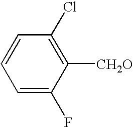 | OMe | H | H | H | 1 |
| 134 | CH₃O | CH₃O | 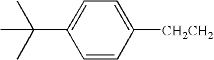 | Cl | O(CH₂)₇CH₃ | OMe | H | H | H | 1 |
| 135 | CH₃O | CH₃O | 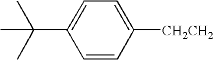 | Cl | 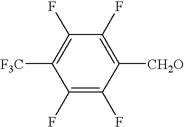 | OMe | H | H | H | 2 |
| 136 | CH₃O | CH₃O | 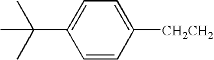 | Cl | 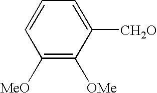 | OMe | H | H | H | 4 |
| 137 | CH₃O | CH₃O | (CH₂)₁₀CH₃ | Cl | H | H | CF₃ | H | H | 1 |
| 138 | CH₃O | CH₃O | CH₃ | Cl | 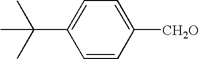 | OMe | H | H | H | 6 |

TABLE 10

| Compound | | | | | | | | | | Relative |
|---|---|---|---|---|---|---|---|---|---|---|
| No. | R¹ | R² | R³ | X | Z¹ | Z² | Z³ | Z⁴ | Z⁵ | Activity |
| 139 | CH₃O | CH₃O | n-Pr | Cl | F | H | H | H | Cl | 6 |
| 140 | CH₃O | CH₃O | Me | Cl | F | H | H | H | Cl | 6 |
| 141 | CH₃O | CH₃O | H | Cl | F | H | H | H | Cl | 6 |
| 142 | CH₃O | CH₃O | H | Cl | H | H | t-Bu | H | H | 6 |
| 143 | CH₃O | CH₃O | Me | Cl | H | H | t-Bu | H | H | 6 |
| 144 | CH₃O | CH₃O | n-Pr | Cl | H | H | t-Bu | H | H | 6 |
| 145 | CH₃O | CH₃O | (CH₂)₅CH₃ | Cl | H | H | t-Bu | H | H | 4 |
| 146 | CH₃O | CH₃O | (CH₂)₁₀CH₃ | Cl | H | H | t-Bu | H | H | 2 |
| 147 | CH₃O | CH₃O | (CH₂)₁₄CH₃ | Cl | H | H | t-Bu | H | H | 1 |
| 148 | CH₃O | CH₃O | 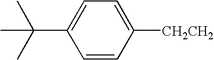 | Cl | O(CH₂)₇(CH)₃ | OMe | H | H | H | 1 |
| 149 | CH₃O | CH₃O | 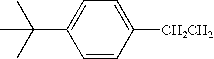 | Cl | 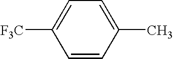 | OMe | H | H | H | 1 |
| 150 | CH₃O | CH₃O | 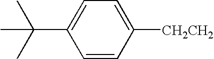 | Cl | F | H | H | H | Cl | 1 |
| 151 | CH₃O | CH₃O | 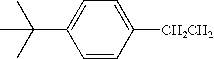 | Cl | F | F | CF₃ | F | F | 1 |

The compounds represented by the above chemical formula (I), wherein R¹ and R² represent each independently methoxy group; R³ represents C₇-C₁₅ alkyl group; Z represents substituted benzyl group, are preferred in an aspect of the pharmaceutical efficacy.

Yet another object of the present invention, is to provide a pharmaceutical composition which contains pharmaceutically effective amount of 3,4-dihydroisoquinolinium salt derivatives of above chemical formula (I).

A still another object of the present invention, is to provide a pharmaceutical c composition which contains pharmaceutically effective amount of isoquinolinium salt derivatives of above chemical formula (II).

Such compositions may be prepared to tablet, syrup, injection or ointment, and may also be administered by oral delivery, injection, vaginal delivery, dermal application. The effective dosage may be varied within the activity range for antifungal or the activity range for hypercholesterolemia and hyperlipidemia depend on the sort or the amount of the above useful excipient or vehicle.

A further object of the present invention is to provide a process for preparing 3,4-dihydroisoquinolinium salt derivatives which comprises □) a step for preparing a compound of the following chemical formula (VI) by reacting a compound of the following chemical formula (III) with a compound of the following chemical formula (IV); □) a step for preparing a compound of the following chemical formula (VII) by reacting a compound of the chemical formula (VI) which is obtained from the above step □) with acyl halide; and □) a step for reacting a compound of the chemical formula (VII) which is obtained from the above step □) in the presence of a catalyst:

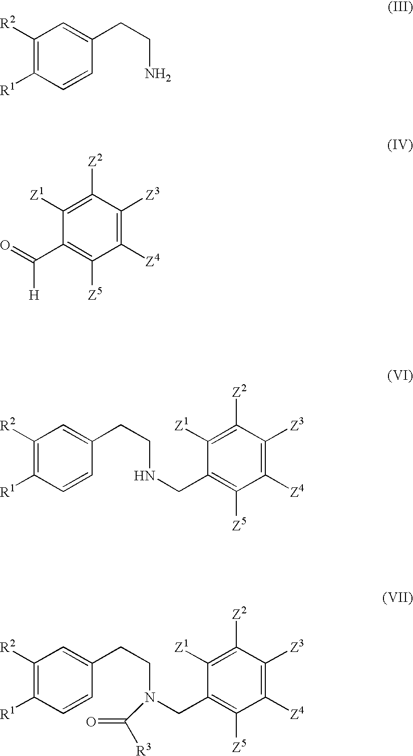

wherein R¹, R², R³, Z¹, Z², Z³, Z⁴, Z⁵ and X⁻ are as defined above.

New compounds which is indicated as above chemical formula (I) according to the present invention can be prepared by a process of the following reaction scheme 1.

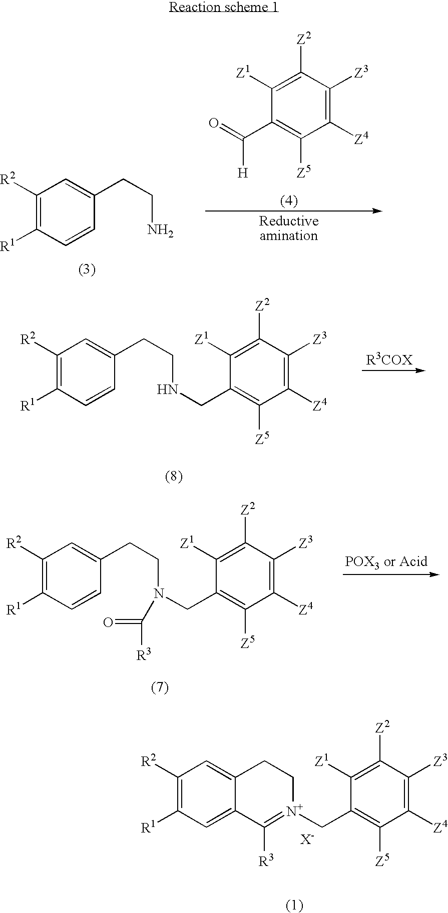

In the above reaction scheme 1, 1.0 mole of substituted phenylethylamine represented by the above chemical formula (III) in methanol solvent and 1.0 mole of substituted benzaldehyde of chemical formula (IV) were heated and cooled to room temperature. Then, 0.5˜1.2 mole of sodium borohydride (NaBH₄) was added to the resulting product, thus reductive amination reaction occurs to prepare a secondary amine represented by the chemical formula (VI). The above secondary amine thus obtained was reacted with 1.0-1.2 mole of acyl halide (R³COX) in an organic solvent to prepare the amide presented by chemical formula (VII). Then, the resulting mixture was reacted in a presence of phosperousoxyhalide, inorganic acid, or Lewis acid to prepare the compounds represented by the above chemical compound (I).

A still further object of the present invention is to provide a process for preparing 3,4-dihydroisoquinolinium salt derivatives which comprises □) a step for preparing a compound of the following chemical formula (VI) by reacting a compound of the following chemical formula (III) with a compound of the following chemical formula (V); □) a step for preparing a compound the following chemical formula (VII) by reacting a compound of the chemical formula (VI) which is obtained from the above step □) with acyl halide; and □) a step for reacting a compound of the chemical formula (VII) which is obtained from the above step □) in the presence of a catalyst:

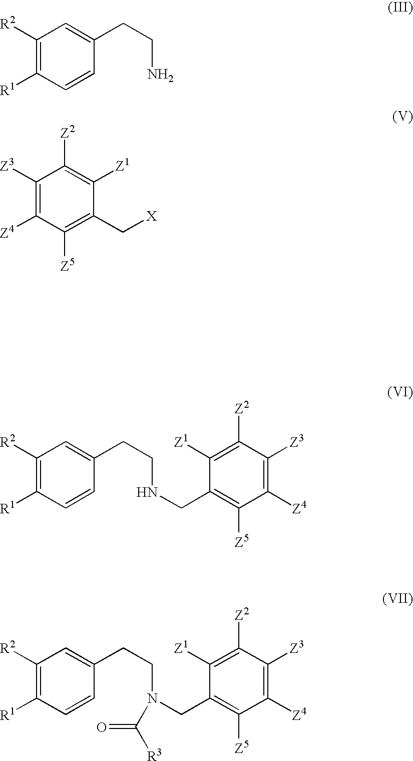

wherein, R¹, R², R³, Z¹, Z², Z³, Z⁴, Z⁵ and X⁻ are as defined above.

The compounds represented by the chemical formula (VI) according to the Sheme 1 may also be synthesized under the different reaction condition according to the Scheme 2 below.

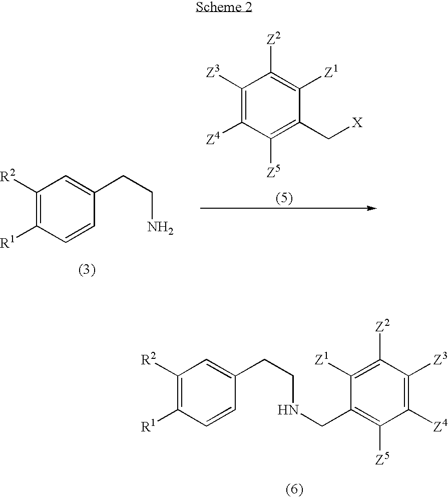

A still further object of the present invention is to provide a process for preparing 3,4-dihydroisoquinolinium salt derivatives which comprises □) a step for preparing a compound of the following chemical formula (□) by reacting a compound of the following chemical formula (III) with acyl halide; □) a step for preparing a compound the following chemical formula (IX) by reacting a compound of the chemical formula (□) which is obtained from the above step □) in the presence of a catalyst; and □) a step for reacting a compound of the chemical formula (IX) which is obtained from the above step □) with a compound of the following chemical formula (V):

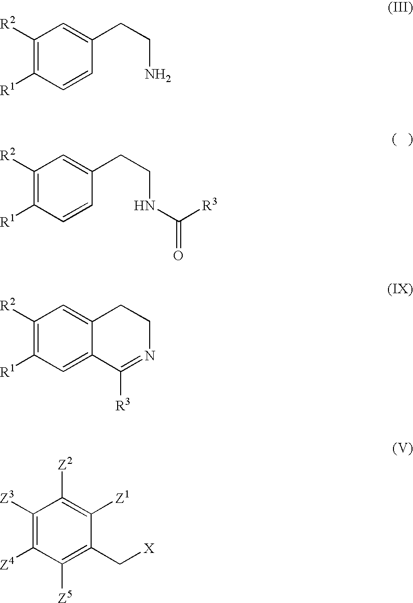

wherein, R¹, R², R³, Z¹, Z², Z³, Z⁴, Z⁵ and X⁻ are as defined above.

Novel compounds represented by the chemical formula (I) according to the present invention may also be synthesized under the different reaction condition according to the Scheme 3 below.

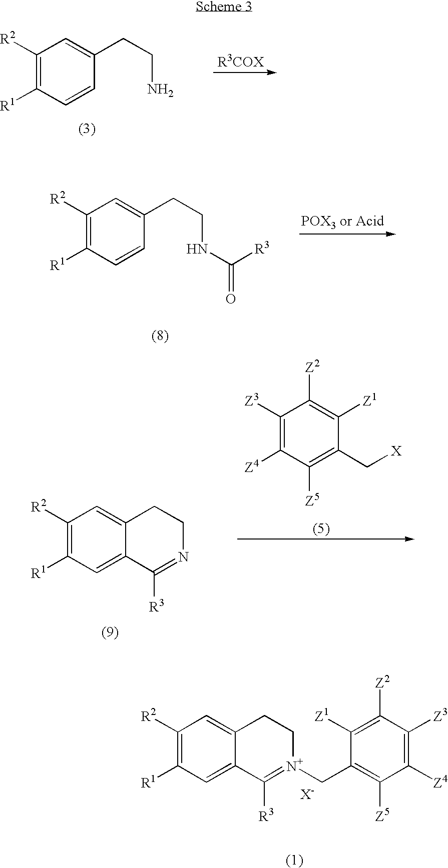

A still further object of the present invention is to provide a process for preparing isoquinolinium salt derivatives by reacting a compound of the following chemical formula (X) with a compound of the following chemical formula (V):

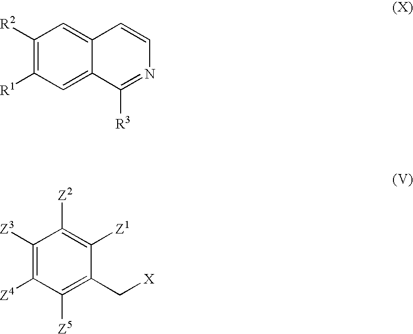

wherein, R¹, R², R³, Z¹, Z², Z³, Z⁴, Z⁵ and X⁻ are as defined above.

Novel compounds represented by the chemical formula (II) according to the present invention may also be synthesized under the different reaction condition according to the Scheme 4 below.

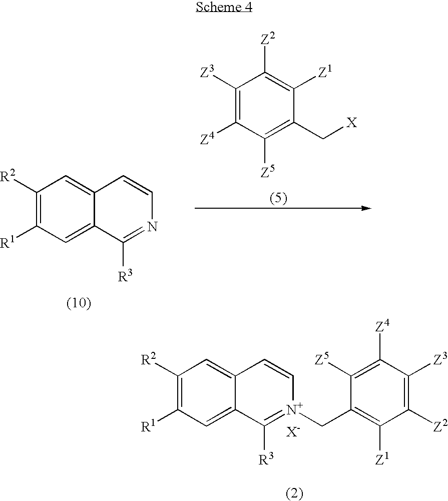

Hereinafter, the preparation processes of the compound of the present invention will be described in greater detail with reference to the following examples. The examples are given only for illustration of the present invention and not to be limiting the present invention. In the following examples, the compound Nos. indicate the Numbers of compounds described in Table 1 to Table 10.

### EXAMPLE 1

### Preparation of 2-(2-fluorophenyl)methyl-3,4-dihydro-6,7-dimethoxy-isoquinolinium chloride (Compound No. 1)

To a 250 ml of methanol solution containing 14.50 g of 3,4-dimethoxyphenethylamine, was added 10.43 g of 2-fluorobenzealdehyde and was heated for 2-3 under reflux. Then, 3.03 g of sodium borohydride was added slowly to the reaction mixture obtained in the above in ice bath, and the resulted mixture was stirred for 1 hour at room temperature and methanol was removed from the reaction mixture under reduced pressure.

The concentrated reaction mixture was suspended into 200 ml of dichloromethane and 200 ml of distilled water was added to the suspension. The phases were separated to obtain organic phase. The aqueous phase was extracted twice with 200 ml of dichloromethane, and the combined organic phase was dried over MgSO₄, filtered and then concentrated under reduced pressure to provide N-(2′-fluorophenyl)methyl-3,4-dimethoxyphenethylamine, quantitatively.

1.16 G of the amine thus obtained was dissolved in 25 ml of 1,2-dichloroethane, and 0.44 g of ethyl formate was added slowly thereto to proceed the reaction at room temperature for about 1 hour. The resulting mixture was concentrated under reduced pressure to produce the amide intermediate.

The crude intermediate was dissolved in 25 ml of acetonitrile and 0.56 ml of phosphoryl chloride was also added to the solution. Then, the mixture was heated for 8 hours under reflux and then concentrated under reduced pressure and separated through the silica-gel column chromatography eluting with dichloromethane and methanol (10:1), to obtain 0.98 g of solid compound, 2-(2-fluorophenyl)methyl-3,4-dihydro-6,7-dimethoxyisoquinolinium chloride (m.p. 188˜189° C.).

¹H-NMR (CDCl₃, 300 MHz): δ 3.18 (t, 2H), 3.94 (s, 3H), 3.98 (s, 3H), 4.01 (br t, 2H), 5.44 (s, 2H), 6.81 (s, 1H), 7.11 (t, 1H), 7.24 (t, 1H), 7.38-7.43 (m, 1H), 7.57 (s, 1H), 7.79 (t, 1H), 9.81 (s, 1H)

### EXAMPLE 2

### Preparation of 1-methyl-2-(2-fluorophenyl)methyl-3,4-dihydro-6,7-dimethoxy-isoquinolinium chloride (Compound No. 2)

To a 25 ml solution of 1,2-dichloroethane containing 1.01 g of N-(2-fluorophenyl)methyl-3,4-dimethoxyphenethylamine was added 0.50 ml of tri-ethylamine and followed by dropwise addition of 0.26 ml of acetyl chloride at 0° C. The reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.46 ml of phosphoryl chloride was added thereto, and the reaction mixture was heated for 8 hours under reflux and cooled to room temperature. The solvent was removed under reduced pressure, and the reaction mixture was separated through silica-gel column chromatography eluting with dichloromethane and methanol (10:1, to give 1.02 g of solid compound, 1-methyl-2-(2-fluorophenyl)methyl-3,4-dihydro-6,7-dimethoxyisoquinolinium chloride.

¹H-NMR (CDCl₃, 300 MHz): δ 3.03 (s, 3H), 3.08 (t, 2H), 3.96 (s, 3H), 3.99 (s, 3H), 4.01 (br t, 2H), 5.35 (s, 2H), 6.82 (s, 1H), 7.12 (dt, 1H), 7.26 (dt, 1H), 7.32 (s, 1H), 7.42-7.44 (m, 1H), 7.58 (dt, 1H)

### EXAMPLE 3

### Preparation of 1-chloromethyl-2-(2-fluorophenyl)methyl-3,4-dihydro-6,7-dimethoxy-isoquinolinium chloride (Compound No. 3)

Chloroacetyle chloride, instead of acetyl chloride of Example 2, was treated by the same process described in Example 2, to give 0.84 g of oily compound, 1-chloromethyl-2-(2-fluorophenyl)methyl-3,4-dihydro-6,7-dimethoxyisoquinolinium chloride.

¹H-NMR (CDCl₃, 300 MHz): δ 3.03 (s, 3H), 3.08 (t, 2H), 3.96 (s, 3H), 3.99 (s, 3H), 4.01 (br t, 2H), 5.35 (s, 2H), 6.82 (s, 1H), 7.12 (dt, 1H), 7.26 (dt, 1H), 7.32 (s, 1H), 7.42-7.44 (m, 1H), 7.58 (dt, 1H)

### EXAMPLE 4

### Preparation of 1-ethyl-2-(2-fluorophenyl)methyl-3,4-dihydro-6,7-dimethoxy-isoquinolinium chloride (Compound No. 4)

Propionyl chloride, instead of acetyl chloride of Example 2, was treated by the same process described in Example 2, to give 0.96 g of solid compound, 1-ethyl-2-(2-fluorophenyl)methyl-3,4-dihydro-6,7-dimethoxyiso-quinolinium chloride (m.p. 173° C.).

¹H-NMR (CDCl₃, 300 MHz): δ 3.03 (s, 3H), 3.08 (t, 2H), 3.96 (s, 3H), 3.99 (s, 3H), 4.01 (br t, 2H), 5.35 (s, 2H), 6.82 (s, 1H), 7.12 (dt, 1H), 7.26 (dt, 1H), 7.32 (s, 1H), 7.42-7.44 (m, 1H), 7.58 (dt, 1H)

### EXAMPLE 5

### Preparation of 1-propyl-2-(2-fluorophenyl)methyl-3,4-dihydro-6,7-dimethoxyisoquinolinium chloride (Compound No. 5)

Butyryl chloride, instead of acetyl chloride of Example 2, was treated by the same process described in Example 2, to give 1.12 g of oily compound, 1-propyl-2-(2-fluorophenyl)methyl-3,4-dihydro-6,7-dimethoxyisoquinolinium chloride.

¹H-NMR (CDCl₃, 300 MHz): δ 1.06 (t, 3H), 1.32 (m, 2H), 1.65 (m, 2H), 3.16 (t, 2H), 3.35 (t, 3H), 3.96 (s, 3H), 4.06 (s, 3H), 4.08 (t, 2H), 5.53 (s, 2H), 6.94 (s, 1H), 7.10 (t, 1H), 7.23-7.32 (m, 2H), 7.38-7.42 (m, 1H), 7.75 (t, 1H)

### EXAMPLE 6

### Preparation of 1-i-propyl-2-(2-fluorophenyl)methyl-3,4-dihydro-6,7-dimethoxyisoquinolinium chloride (Compound No. 6)

i-Butyryl chloride, instead of acetyl chloride of Example 2, was treated by the same process described in Example 2, to give 1.25 g of solid compound, 1-i-propyl-2-(2-fluorophenyl)methyl-3,4-dihydro-6,7-dimethoxyiso-quinolinium chloride (m.p. 122° C.).

¹H-NMR (CDCl₃, 300 MHz): δ 1.63 (s, 3H), 1.65 (s, 3H), 3.02 (t, 2H), 3.92 (br t, 2H), 3.94 (s, 3H), 4.00 (s, 3H), 5.59 (s, 2H), 6.83 (s, 2H), 7.09-7.15 (t, 1H), 7.26-7.30 (t, 1H), 7.38 (s, 1H), 7.43-7.45 (t, 1H), 7.64-7.70 (t, 1H)

### EXAMPLE 7

### Preparation of 1-(3-chloropropyl)-2-(2-fluorophenyl)methyl-3,4-dihydro-6,7-dimethoxyisoquinolinium chloride (Compound No. 7)

4-Chlorobutyryl chloride, instead of acetyl chloride of Example 2, was treated by the same process described in Example 2, to give 1.15 g of oily compound, 1-(3-chloropropyl)-2-(2-fluorophenyl)methyl-3,4-dihydro-6,7-dimethoxyisoquinolinium chloride.

¹H-NMR (CDCl₃, 300 MHz): δ 2.29 (br t, 2H), 3.18 (t, 2H), 3.68 (br t, 2H), 3.84 (t, 2H), 3.98 (s, 3H), 4.01 (s, 3H), 4.09 (t, 2H), 5.73 (s, 2H), 6.91 (s 2H), 7.11 (t, 1H), 7.24 (t, 1H), 7.39-7.44 (m, 1H), 7.49 (s, 1H), 7.74 (t, 1H)

### EXAMPLE 8

### Preparation of 1-(2-methyl)propyl-2-(2-fluorophenyl)methyl-3,4-dihydro-6,7-dimethoxyisoquinolinium chloride (Compound No. 8)

Isovaleryl chloride, instead of acetyl chloride of Example 2, was treated by the same process described in Example 2, to give 0.91 g of oily compound, 1-(2-methyl)propyl-2-(2-fluorophenyl)methyl-3,4-dihydro-6,7-dimethoxyisoquinolinium chloride.

¹H-NMR (CDCl₃, 300 MHz): δ 0.99 (s, 3H), 1.01 (s, 3H), 2.02-2.17 (m, 1H), 3.23 (br t, 2H), 3.36 (m, 2H), 3.88 (br t, 2H), 3.99 (s, 3H), 4.02 (s, 3H), 5.61 (s, 2H), 7.02 (s, 1H), 7.11 (t, 1H), 7.22-7.28 (m, 1H), 7.38 (s, 1H), 7.39-7.44 (m, 1H), 7.81 (m, 1H)

### EXAMPLE 9

### Preparation of 1-n-pentyl-2-(2-fluorophenyl)methyl-3,4-dihydro-6,7-dimethoxyisoquinolinium chloride (Compound No. 9)

Caproyl chloride, instead of acetyl chloride of Example 2, was treated by the same process described in Example 2, to give 1.10 g of oily compound, 1-n-pentyl-2-(2-fluorophenyl)methyl-3,4-dihydro-6,7-dimethoxyisoquinolinium chloride.

¹H-NMR (CDCl₃, 300 MHz): δ 0.88 (t, 3H), 1.31-1.47 (m, 4H), 1.55-1.62 (m, 2H), 3.16 (t, 2H), 3.21 (t, 2H), 3.95 (s, 3H), 4.01 (s, 3H), 4.20 (t, 2H), 5.43 (s, 2H), 6.88 (s, 1H), 7.12 (t, 1H), 7.25-7.30 (m, 2H), 7.43 (q, 1H), 7.67 (dt, 1H)

### EXAMPLE 10

### Preparation of 1-n-hexyl-2-(2-fluorophenyl)methyl-3,4-dihydro-6,7-dimethoxyisoquinolinium chloride (Compound No. 10)

Heptanoyl chloride, instead of acetyl chloride of Example 2, was treated by the same process described in Example 2, to give 1.13 g of oily compound, 1-n-hexyl-2-(2-fluorophenyl)methyl-3,4-dihydro-6,7-dimethoxy-isoquinolinium chloride.

¹H-NMR (CDCl₃, 300 MHz): δ 0.86 (t, 3H), 1.26 (m, 4H), 1.45 (m, 2H), 1.51 (m, 2H), 3.14 (br t, 2H), 3.30 (m, 2H), 3.94 (s, 3H), 3.99 (s, 3H), 4.12 (br t, 2H), 5.42 (s, 2H), 6.92 (s, 1H), 7.11 (m, 1H), 7.25-7.30 (m, 2H), 7.38-7.46 (m, 1H), 7.65 (t, 1H)

### EXAMPLE 11

### Preparation of 1-n-heptyl-2-(2-fluorophenyl)methyl-3,4-dihydro-6,7-dimethoxyisoquinolinium chloride (Compound No. 11)

Octanoyl chloride, instead of acetyl chloride of Example 2, was treated by the same process described in Example 2, to give 1.06 g of solid compound, 1-n-heptyl-2-(2-fluorophenyl)methyl-3,4-dihydro-6,7-dimethoxyisoquinolinium chloride (m.p. 112° C.).

¹H-NMR (CDCl₃, 300 MHz): δ 0.87 (t, 3H), 1.26-1.29 (m, 6H), 1.46 (m, 2H), 1.64 (m, 2H), 3.16 (t, 2H), 3.32 (t, 2H), 3.94 (s, 3H), 4.00 (s, 3H), 4.18 (t, 2H), 5.75 (s, 2H), 6.80 (s, 1H), 7.07-7.13 (dt, 1H), 7.22 (s, 1H), 7.25-7.29 (dt, 1H), 7.41-7.43 (m, 1H), 7.92-7.97 (dt, 1H)

### EXAMPLE 12

### Preparation of 1-n-undecyl-2-(2-fluorophenyl)methyl-3,4-dihydro-6,7-dimethoxyisoquinolinium chloride (Compound No. 12)

Lauroyl chloride, instead of acetyl chloride of Example 2, was treated by the same process described in Example 2, to give 1.16 g of oily compound, 1-n-undecyl-2-(2-fluorophenyl)methyl-3,4-dihydro-6,7-dimethoxyisoquinolinium chloride.

¹H-NMR (CDCl₃, 300 MHz): δ 0.88 (t, 3H), 1.24 (m, 14H), 1.42 (m, 2H), 1.59 (m, 2H), 3.17 (br t, 2H), 3.32 (br t, 2H), 3.95 (s, 3H), 4.01 (s, 3H), 4.07 (br t, 2H), 5.51 (s, 2H), 6.89 (s, 1H), 7.12 (t, 1H), 7.25-7.29 (m, 2H), 7.42-7.44 (m, 1H), 7.74 (m, 1H)

### EXAMPLE 13

### Preparation of 1-n-pentadecyl-2-(2-fluorophenyl)methyl-3,4-dihydro-6,7-dimethoxyisoquinolinium chloride (Compound No. 13)

Palmitoyl chloride, instead of acetyl chloride of Example 2, was treated by the same process described in Example 2, to give 1.07 g of oily compound, 1-n-pentadecyl-2-(2-fluorophenyl)methyl-3,4-dihydro-6,7-dimethoxy-isoquinolinium chloride.

¹H-NMR (CDCl₃, 300 MHz): δ 0.88 (t, 3H), 1.22 (m, 22H), 1.43 (m, 2H), 1.59 (m, 2H), 3.18 (br t, 2H), 3.32 (m 2H), 3.95 (s, 3H), 4.01 (s, 3H), 4.12 (br t, 2H), 5.58 (s, 2H), 6.88 (s, 1H), 7.11 (t, 1H), 7.25-7.28 (m, 2H), 7.42-7.44 (m, 1H), 7.81 (m, 1H)

### EXAMPLE 14

### Preparation of 1-(2-fluorophenyl)-2-(2-fluorophenyl)methyl-3,4-dihydro-6,7-dimethoxyisoquinolinium chloride (Compound No. 14)

2-Fluorobenzoyl chloride, instead of acetyl chloride of Example 2, was treated by the same process described in Example 2, to give 1.03 g of oily compound, 1-(2-fluorophenyl)-2-(2-fluorophenyl)methyl-3,4-dihydro-6,7-dimethoxyisoquinolinium chloride.

¹H-NMR (CDCl₃, 300 MHz): δ 3.16-3.19 (m, 1H), 3.30-3.43 (m, 1H), 3.62 (s, 3H), 4.02 (s, 3H), 4.07-4.15 (m, 1H), 4.76-4.85 (m, 1H), 5.35 (d, J=12 Hz, 1H), 5.54 (d, J=12 Hz, 1H), 6.44 (s, 1H), 6.99 (s, 1H), 7.07 (t, 1H), 7.17 (t, 1H), 7.30 (t, 1H), 7.38-7.43 (q, 1H), 7.45-7.53 (m, 2H), 7.68-7.74 (m, 1H), 7.85 (t, 1H)

### EXAMPLE 15

### Preparation of 1-(2,3-difluorophenyl)-2-(2-fluorophenyl)methyl-3,4-dihydro-6,7-dimethoxyisoquinolinium chloride (Compound No. 15)

2,3-Difluorobenzoyl chloride, instead of acetyl chloride of Example 2, was treated by the same process described in Example 2, to give 1.03 g of oily compound, 1-(2,3-difluorophenyl)-2-(2-fluorophenyl)methyl-3,4-dihydro-6,7-dimethoxyisoquinolinium chloride.

¹H-NMR (CDCl₃, 300 MHz): δ 3.18 (m, 1H), 3.36 (m, 1H), 3.64 (s, 3H), 4.03 (s, 3H), 4.16 (m, 1H), 4.68 (m, 1H), 5.30 (dd, 2H), 6.42 (s, 1H), 7.04-7.10 (m, 2H), 7.17 (t, 1H), 7.36-7.43 (m, 2H), 7.55-7.56 (m, 2H), 8.10 (m, 1H)

### EXAMPLE 16

### Preparation of 1-(2,4-difluorophenyl)-2-(2-fluorophenyl)methyl-3,4-dihydro-6,7-dimethoxyisoquinolinium chloride (Compound No. 16)

2,4-Difluorobenzoyl chloride, instead of acetyl chloride of Example 2, was treated by the same process described in Example 2, to give 1.10 g of oily compound, 1-(2,4-difluorophenyl)-2-(2-fluorophenyl)methyl-3,4-dihydro-6,7-dimethoxyisoquinolinium chloride.

¹H-NMR (CDCl₃, 300 MHz): δ 3.10 (m, 1H), 3.32 (m, 1H), 3.64 (s, 3H), 4.01 (s, 3H), 4.11 (m, 1H), 4.71 (m, 1H), 5.31 (dd, 2H), 6.42 (s, 1H), 6.97-7.06 (m, 3H), 7.17 (t, 1H), 7.29-7.37 (m, 2H), 7.46 (t, 1H), 8.43-8.45 (m, 1H)

### EXAMPLE 17

### Preparation of 1-(3,4-difluorophenyl)-2-(2-fluorophenyl)methyl-3,4-dihydro-6,7-dimethoxyisoquinolinium chloride (Compound No. 17)

3,4-Difluorobenzoyl chloride, instead of acetyl chloride of Example 2, was treated by the same process described in Example 2, to give 1.11 g of solid compound, 1-(3,4-difluorophenyl)-2-(2-fluorophenyl)methyl-3,4-dihydro-6,7-dimethoxyisoquinolinium chloride (m.p. 114˜115° C.).

¹H-NMR (CDCl₃, 300 MHz): 3.21 (t, 2H), 3.65 (s, 3H), 4.03 (s, 3H), 4.38 (t, 2H), 5.40 (s, 2H), 6.38 (s, 1H), 7.04 (s, 1H), 7.10 (t, 1H), 7.22 (t, 1H), 7.40-7.53 (m, 3H), 7.82-7.87 (m, 1H), 8.02 (t, 1H)

### EXAMPLE 18

### Preparation of 1-(3,5-difluorophenyl)-2-(2-fluorophenyl)methyl-3,4-dihydro-6,7-dimethoxyisoquinolinium chloride (Compound No. 18)

3,5-Difluorobenzoyl chloride, instead of acetyl chloride of Example 2, was treated by the same process described in Example 2, to give 0.96 g of solid compound, 1-(3,5-difluorophenyl)-2-(2-fluorophenyl)methyl-3,4-dihydro-6,7-dimethoxyisoquinolinium chloride (m.p. 118° C.).

¹H-NMR (CDCl₃, 300 MHz): δ 3.26 (br t, 2H), 3.66 (s, 3H), 4.04 (s, 3H), 4.34 (br t, 2H), 5.32 (s, 2H), 6.39 (s, 1H), 7.03-7.15 (m, 3H), 7.23 (s, 1H), 7.39-7.48 (m, 1H), 7.52-7.62 (m, 2H), 9.06 (m, 1H)

### EXAMPLE 19

### Preparation of 1-(3-chlorophenyl)-2-(2-fluorophenyl)methyl-3,4-dihydro-6,7-dimethoxyisoquinolinium chloride (Compound No. 19)

3-Chlorobenzoyl chloride, instead of acetyl chloride of Example 2, was treated by the same process described in Example 2, to give 1.21 g of 1-(3-chlorophenyl)-2-(2-fluorophenyl)methyl-3,4-dihydro-6,7-dimethoxyisoquinolinium chloride.

¹H-NMR (CDCl₃, 300 MHz): δ 3.19-3.27 (m, 2H), 3.62 (s, 3H), 4.02 (s, 3H), 4.27-4.32 (m, 1H), 4.51-4.55 (m, 1H), 5.45 (dd, 2H), 6.38 (s, 1H), 6.98 (s, 1H), 7.08 (t, 1H), 7.20 (t, 1H), 7.39-7.42 (m, 1H), 7.53 (t, 1H), 7.60-7.65 (m, 2H), 7.76 (s, 1H), 8.04-8.06 (m, 1H)

### EXAMPLE 20

### Preparation of 1-(4-chlorophenyl)-2-(2-fluorophenyl)methyl-3,4-dihydro-6,7-dimethoxyisoquinolinium chloride (Compound No. 20)

4-Chlorobenzoyl chloride, instead of acetyl chloride of Example 2, was treated by the same process described in Example 2, to give 1.23 g of 1-(4-chlorophenyl)-2-(2-fluorophenyl)methyl-3,4-dihydro-6,7-dimethoxyiso-quinolinium chloride.

¹H-NMR (CDCl₃, 300 MHz): δ 3.16 (t, 2H), 3.63 (s, 3H), 4.01 (s, 3H), 4.39 (t, 2H), 5.41 (s, 2H), 6.40 (s, 1H), 6.89 (s, 1H), 7.04 (t, 1H), 7.21 (t, 1H), 7.37-7.40 (m, 1H), 7.55-7.65 (m, 3H), 7.87-7.90 (m, 2H)

### EXAMPLE 21

### Preparation of 1-(4-n-butylphenyl)-2-(2-fluorophenyl)methyl-3,4-dihydro-6,7-dimethoxyisoquinolinium chloride (Compound No. 21)

4-n-Butylbenzoyl chloride, instead of acetyl chloride of Example 2, was treated by the same process described in Example 2, to give 1.31 g of solid compound, 1-(4-n-butylphenyl)-2-(2-fluorophenyl)methyl-3,4-dihydro-6,7-dimethoxyisoquinolinium chloride (m.p. 147˜149° C.).

¹H-NMR (CDCl₃, 300 MHz): δ 0.96 (t, 3H), 1.35-1.40 (m, 2H), 1.63-1.67 (m, 2H), 2.74 (t, 2H), 3.18 (br t, 2H), 3.60 (s, 3H), 4.00 (s, 3H), 4.41 (br t, 2H), 5.53 (s, 2H), 6.44 (s, 1H), 6.85 (s, 1H), 7.03 (t, 1H), 7.18 (t, 1H), 7.36-7.38 (m, 1H), 7.44 (d, J=6 Hz, 2H), 7.62 (t, 1H), 7.73 (d, J=6 Hz, 2H)

### EXAMPLE 22

### Preparation of 1-(4-t-butylphenyl)-2-(2-fluorophenyl)methyl-3,4-dihydro-6,7-dimethoxyisoquinolinium chloride (Compound No. 22)

4-t-Butylbenzoyl chloride, instead of acetyl chloride of Example 2, was treated by the same process described in Example 2, to give 1.45 g of solid compound, 1-(4-t-butylphenyl)-2-(2-fluorophenyl)methyl-3,4-dihydro-6,7-dimethoxyisoquinolinium chloride (m.p. 125° C.).

¹H-NMR (CDCl₃, 300 MHz): δ 1.39 (s, 9H), 3.15 (t, 2H), 3.62 (s, 3H), 4.02 (s, 3H), 4.21 (br t, 2H), 5.30 (s, 2H), 6.46 (s, 1H), 6.88 (s, 1H), 7.02-7.08 (dt, 1H), 7.17-7.22 (dt, 1H), 7.37-7.39 (m, 1H), 7.48-7.54 (dt, 1H), 7.60 (d, J=9 Hz, 2H), 7.66 (d, J=9 Hz, 2H)

### EXAMPLE 23

### Preparation of 1-(3-trifluoromethylphenyl)-2-(2-fluorophenyl) methyl-3,4-dihydro-6,7-dimethoxyisoquinolinium chloride (Compound No. 23)

4-Trifluoromethylbenzoyl chloride, instead of acetyl chloride of Example 2, was treated by the same process described in Example 2, to give 1.11 g of oily compound, 1-(3-trifluoromethylphenyl)-2-(2-fluorophenyl)methyl-3,4-dihydro-6,7-dimethoxyisoquinolinium chloride.

¹H-NMR (CDCl₃, 300 MHz): δ 3.17 (m, 1H), 3.27 (m, 1H), 3.58 (s, 3H), 4.02 (s, 3H), 4.13 (m, 1H), 4.71 (m, 1H), 5.25 (d, 1H), 5.48 (d, 1H), 6.30 (s, 1H), 6.89 (s, 1H), 7.06 (t, 1H), 7.19 (t, 1H), 7.34-7.42 (m, 1H), 7.53 (m, 1H), 7.79 (s, 1H), 7.87-8.01 (m, 2H), 8.61 (m, 1H)

### EXAMPLE 24

### Preparation of 1-(2-fluorophenyl)methyl-2-(2-fluorophenyl)methyl-3,4-dihydro-6,7-dimethoxyisoquinolinium chloride (Compound No. 24)

(2-Fluorophenyl)acetyl chloride, instead of acetyl chloride of Example 2, was treated by the same process described in Example 2, to give 1.03 g of oily compound, 1-(2-fluorophenyl)methyl-2-(2-fluorophenyl)methyl-3,4-dihydro-6,7-dimethoxyisoquinolinium chloride.

¹H-NMR (CDCl₃, 300 MHz): δ 3.23 (t, 2H), 3.79 (s, 3H), 3.99 (s, 3H), 4.20 (t, 2H), 4.99 (s, 2H), 5.70 (s, 2H), 6.77-6.84 (m, 2H), 6.93-7.10 (m, 3H), 7.16 (dt, 1H), 7.22-7.30 (m, 2H), 7.33-7.38 (m, 1H), 7.65 (dt, 1H)

### EXAMPLE 25

### Preparation of 1-(2,4-dichlorophenyl)methyl-2-(2-fluorophenyl)methyl-3,4-dihydro-6,7-dimethoxyisoquinolinium chloride (Compound No. 25)

(2,4-Dichlorophenyl)acetyl chloride, instead of acetyl chloride of Example 2, was treated by the same process described in Example 2, to give 1.24 g of solid compound, 1-(2,4-dichlorophenyl)methyl-2-(2-fluorophenyl)methyl-3,4-dihydro-6,7-dimethoxyisoquinolinium chloride (m.p. 182° C.).

¹H-NMR (CDCl₃, 300 MHz): δ 3.21 (br t, 2H), 3.81 (s, 3H), 3.97 (s, 3H), 4.21 (br t, 2H), 4.94 (s, 2H), 5.75 (s, 2H), 6.73-6.77 (m, 1H), 6.78 (s, 1H), 6.89 (t, 1H), 7.10 (t, 1H), 7.19 (t, 1H), 7.29 (s, 1H), 7.39-7.42 (m, 1H), 7.54-7.56 (m, 1H), 7.71 (t, 1H)

### EXAMPLE 26

### Preparation of 1-methyl-2-(4-trifluoromethylphenyl)methyl-3,4-dihydro-6,7-dimethoxyisoquinolinium chloride (Compound No. 26)

To a 250 ml of methanol solution containing 14.50 g of 3,4-dimethoxyphenethylamine was added 13.9 g of a,a,a-trifluoro tolualdehyde, and was heated for 2-3 under reflux. Then, 3.03 g of sodium borohydride was added slowly to the reaction mixture obtained in the above in ice bath, and the resulted mixture was stirred for 1 hour at room temperature and methanol was removed from the reaction mixture under reduced pressure

The concentrated reaction mixture was suspended into 200 ml of dichloromethane and 200 ml of distilled water was added to the suspension. The phases were separated to obtain organic phase. The aqueous phase was extracted twice with 200 ml of dichloromethane, and the organic phase was dried over MgSO₄, filtered and then concentrated under reduced pressure to prepare N-(4′-trifluoromethylphenyl)methyl-3,4-dimethoxyphenethyl amine, quantitatively.

To a 25 ml 1,2-dichloroethane solution of 1.36 g of the amine thus obtained was slowly added 0.56 ml of triethylamine. Then, 0.26 ml of acetyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure and the reaction mixture was separated through silica-gel column chromatography eluting with dichloromethane and methanol (10:1), to give 1.21 g of solid compound, 1-methyl-2-(4-trifluoromethylphenyl)methyl-3,4-dihydro-6,7-dimethoxyisoquinolinium chloride (m.p. 115˜120° C.).

¹H-NMR (CDCl₃, 300 MHz): δ 3.01 (s, 3H), 3.13 (br t, 2H), 3.95 (s, 3H), 3.99 (s, 3H), 4.06 (br t, 2H), 5.50 (s, 2H), 6.82 (s, 1H), 9.34 (s, 1H), 7.50 (d, 2H), 7.66 (d, 2H)

### EXAMPLE 27

### Preparation of 1-n-pentyl-2-(4-trifluoromethylphenyl)methyl-3,4-dihydro-6,7-dimethoxyisoquinolinium chloride (Compound No. 27)

To a 25 ml 1,2-dichloroethane solution containing 1.36 g of N-(4-trifluoromethylphenyl)methyl-3,4-dimethoxyphenethylamine was added 0.50 ml of triethylamine. Then, 0.65 g of caproyl chloride was added slowly to the mixture at 0° C., and the reaction mixture was stiffed at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 1.10 g of oily compound, 1-n-pentyl-2-(4-trifluoromethylphenyl)methyl-3,4-dihydro-6,7-dimethoxyisoquinolinium chloride.

¹H-NMR (CDCl₃, 300 MHz): δ 0.88 (t, 3H), 1.25-1.46 (m, 4H), 1.72 (m, 2H), 3.21 (t, 2H), 3.29 (t, 2H), 3.96 (s, 3H), 4.02 (s, 3H), 4.20 (t, 2H), 5.81 (s, 2H), 6.85 (s, 1H), 7.27 (s, 1H), 7.59-7.71 (dd, J=7.8 Hz, 4H)

### EXAMPLE 28

### Preparation of 1-n-heptyl-2-(4-trifluoromethylphenyl)methyl-3,4-dihydro-6,7-dimethoxyisoquinolinium chloride (Compound No. 28)

Octanoyl chloride, instead of caproyl chloride of Example 27, was treated by the same process described in Example 27, to give 1.13 g of oily compound, 1-n-heptyl-2-(4-trifluoromethylphenyl)methyl-3,4-dihydro-6,7-dimethoxyisoquinolinium chloride.

¹H-NMR (CDCl₃, 300 MHz): δ 0.81 (t, 3H), 1.16-1.22 (m, 6H), 1.25 (m, 2H), 1.34 (m, 2H), 3.16 (br t, 2H), 3.25 (q, 2H), 3.91 (s, 3H), 3.97 (s, 3H), 4.13 (br t, 2H), 5.55 (s, 2H), 6.93 (s, 1H), 7.28 (s, 1H), 7.54 (d, J=9 Hz, 2H), 7.63 (d, J=9 Hz, 2H)

### EXAMPLE 29

### Preparation of 1-n-undecyl-2-(4-trifluoromethylphenyl)methyl-3,4-dihydro-6,7-dimethoxyisoquinolinium chloride (Compound No. 29)

Lauroyl chloride, instead of caproyl chloride of Example 27, was treated by the same process described in Example 27, to give 1.16 g of oily compound, 1-n-undecyl-2-(4-trifluoromethylphenyl)methyl-3,4-dihydro-6,7-dimethoxyisoquinolinium chloride.

¹H-NMR (CDCl₃, 300 MHz): δ 0.87 (t, 3H), 1.21 (m, 14H), 1.42 (m, 2H), 1.60 (m, 2H), 3.12 (br t, 2H), 3.21 (br t, 2H), 3.93 (s, 3H), 4.00 (s, 3H), 4.04 (br t, 2H), 5.52 (s, 2H), 6.87 (s, 1H), 7.27 (s, 1H), 7.53 (m, 2H), 7.68 (m, 2H)

### EXAMPLE 30

### Preparation of 1-n-pentadecyl-2-(4-trifluoromethylphenyl)methyl-3,4-dihydro-6,7-dimethoxyisoquinolinium chloride (Compound No. 30)

Palmitoyl chloride, instead of caproyl chloride of Example 27, was treated by the same process described in Example 27, to give 1.07 g of oily compound, 1-n-pentadecyl-2-(4-trifluoromethylphenyl)methyl-3,4-dihydro-6,7-dimethoxyisoquinolinium chloride.

¹H-NMR (CDCl₃, 300 MHz): δ 0.88 (t, 3H), 1.25 (m, 22H), 1.46 (m, 2H), 1.66 (m, 2H), 3.17 (br t, 2H), 3.22 (m 2H), 3.96 (s, 3H), 4.02 (s, 3H), 4.10 (br t, 2H), 5.56 (s, 2H), 6.84 (s, 1H), 7.27 (s, 1H), 7.52-7.54 (d, J=6 Hz, 2H), 7.70-7.72 (d, J=6 Hz, 2H)

### EXAMPLE 31

### Preparation of 1-(2-fluorophenyl)-2-(4-trifluoromethylphenyl)methyl-3,4-dihydro-6,7-dimethoxyisoquinolinium chloride (Compound No. 31)

2-Fluorobenzoyl chloride, instead of caproyl chloride of Example 27, was treated by the same process described in Example 27, to give 0.98 g of oily compound, 1-(2-fluorophenyl)-2-(4-trifluoromethylphenyl)methyl-3,4-dihydro-6,7-dimethoxyisoquinolinium chloride.

¹H-NMR (CDCl₃, 300 MHz): δ 3.10 (br t, 2H), 3.61 (s, 3H), 3.99 (br t, 2H), 4.02 (s, 3H), 5.20 (d, J=15 Hz, 1H), 5.51 (d, J=15 Hz, 1H), 6.41 (s, 1H), 6.96 (s, 1H) 7.26-7.34 (m, 1H), 7.51-7.65 (m, 6H), 8.37 (s, 1H)

### EXAMPLE 32

### Preparation of 1-(4-t-butylphenyl)-2-(4-trifluoromethylphenyl)methyl-3,4-dihydro-6,7-dimethoxyisoquinolinium chloride (Compound No. 32)

4-t-Butylbenzoyl chloride, instead of caproyl chloride of Example 27, was treated by the same process described in Example 27, to give 1.32 g of solid compound, 1-(4-t-butylphenyl)-2-(4-trifluoromethylphenyl)methyl-3,4-dihydro-6,7-dimethoxyisoquinolinium chloride (m.p. 119˜124° C.).

¹H-NMR (CDCl₃, 300 MHz): δ 1.38 (s, 9H), 3.20 (br t, 2H), 3.62 (s, 3H), 4.02 (s, 3H), 4.29 (br t, 2H), 5.38 (s, 2H), 6.46 (s, 1H), 6.87 (s, 1H), 7.44 (m, 2H), 7.61-7.68 (m, 6H)

### EXAMPLE 33

### Preparation of 1-methyl-2-(4-methoxyphenyl)methyl-3,4-dihydro-6,7-dimethoxyisoquinolinium chloride (Compound No. 33)

To a 250 ml of methanol solution containing 14.50 g of 3,4-dimethoxyphenethylamine was added 11.4 g of p-anisaldehyde, and was heated for 2-3 under reflux and cooled to room temperature. Then, 3.03 g of sodium borohydride was added slowly to the reaction mixture obtained in the above in ice bath, and the resulted mixture was stirred for 1 hour at room temperature and methanol was removed from the reaction mixture under reduced pressure. The reaction mixture was suspended into 200 ml of dichloromethane and 200 ml of distilled water was added to the suspension. The phases were separated to obtain organic phase. The aqueous phase was extracted twice with 200 ml of dichloromethane, and the organic phase thus separated was dried over MgSO₄, filtered and then concentrated under reduced pressure to prepare N-(4′-methoxyphenyl)methyl-3,4-dimethoxyphenethylamine, quantitatively.

1.36 G of the amine thus obtained was dissolved in 25 ml of 1,2-dichloroethane, and 0.56 ml of triethylamine was added thereto. Then, 0.26 ml of acetyl chloride was added slowly to the mixture at 0° C., and the reaction was proceeded at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1) as an elute solvent, to give 1.06 g of solid compound, 1-methyl-2-(4-methoxyphenyl)methyl-3,4-dihydro-6,7-dimethoxyisoquinolinium chloride.

¹H-NMR (CDCl₃, 300 MHz): δ 3.04 (s, 3H), 3.06 (br t, 2H), 3.78 (s, 3H), 3.95 (s, 3H), 3.96 (s, 3H), 4.01 (br t, 2H), 5.30 (s, 2H), 6.79 (s, 1H), 6.90 (d, 2H), 7.28 (s, 1H), 7.32 (d, 2H)

### EXAMPLE 34

### Preparation of 1-n-pentyl-2-(4-methoxyphenyl)methyl-3,4-dihydro-6,7-dimethoxyisoquinolinium chloride (Compound No. 34)

To a 25 ml of 1,2-dichloroethane solution containing 1.21 g of N-(4-methoxyphenyl)methyl-3,4-dimethoxyphenethylamine was added 0.50 ml of triethylamine. Then, 0.64 g of caproyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1) as an elute solvent, to give 1.10 g of oily compound, 1-n-pentyl-2-(4-methoxyphenyl)methyl-3,4-dihydro-6,7-dimethoxyisoquinolinium chloride.

¹H-NMR (CDCl₃, 300 MHz): δ 0.89 (t, 3H), 1.34-1.47 (m, 4H), 1.67 (m, 2H), 3.14 (t, 2H), 3.31 (t, 2H), 3.81 (s, 3H), 3.96 (s, 3H), 4.00 (s, 3H), 4.09 (t, 2H), 5.40 (s, 2H), 6.90 (s, 1H), 6.92-6.95 (m, 2H), 7.29 (s, 1H), 7.33-7.36 (m, 2H)

### EXAMPLE 35

### Preparation of 1-n-hexyl-2-(4-methoxyphenyl)methyl-3,4-dihydro-6,7-dimethoxyisoquinolinium chloride (Compound No. 35)

397Heptanoyl chloride, instead of caproyl chloride of Example 34, was treated by the same process described in Example 34, to give 1.13 g of oily compound, 1-n-hexyl-2-(4-methylphenyl)methyl-3,4-dihydro-6,7-dimethoxyisoquinolinium chloride.

¹H-NMR (CDCl₃, 300 MHz): δ 0.90 (t, 3H), 1.21-1.44 (m, 6H), 1.73 (m, 2H), 3.17 (br t, 2H), 3.32 (m, 2H), 3.81 (s, 3H), 3.97 (s, 3H), 4.00 (s, 3H), 4.13 (br t, 2H), 5.52 (s, 2H), 6.84 (s, 1H), 6.93-6.95 (d, J=7.8 Hz, 2H), 7.28 (s, 1H), 7.35-7.37 (d, J=7.8 Hz, 2H)

### EXAMPLE 36

### Preparation of 1-n-heptyl-2-(4-methoxyphenyl)methyl-3,4-dihydro-6,7-dimethoxyisoquinolinium chloride (Compound No. 36)

Octanoyl chloride, instead of caproyl chloride of Example 34, was treated by the same process described in Example 34, to give 1.17 g of oily compound, 1-n-heptyl-2-(4-methylphenyl)methyl-3,4-dihydro-6,7-dimethoxyisoquinolinium chloride.

¹H-NMR (CDCl₃, 300 MHz): δ 0.87 (t, 3H), 1.21-1.25 (m, 6H), 1.47 (m, 2H), 1.67 (m, 2H), 3.15 (m, 2H), 3.30 (m, 2H), 3.82 (s, 3H), 3.96 (s, 3H), 4.01 (s, 3H), 4.13 (br t, 2H), 5.16 (s, 2H), 6.91-6.98 (m, 4H), 7.28-7.35 (m, 2H)

### EXAMPLE 37

### Preparation of 1-n-undecyl-2-(4-methoxyphenyl)methyl-3,4-dihydro-6,7-dimethoxyisoquinolinium chloride (Compound No. 37)

Lauroyl chloride, instead of caproyl chloride of Example 34, was treated by the same process described in Example 34, to give 1.16 g of oily compound, 1-n-undecyl-2-(4-methylphenyl)methyl-3,4-dihydro-6,7-dimethoxy-isoquinolinium chloride.

¹H-NMR (CDCl₃, 300 MHz): δ 0.87 (t, 3H), 1.24 (m, 14H), 1.47 (m, 2H), 1.67 (m, 2H), 3.15 (br t, 2H), 3.21 (br t, 2H), 3.81 (s, 3H), 3.95 (s, 3H), 4.00 (s, 3H), 4.12 (br t, 2H), 5.48 (s, 2H), 6.89 (s, 1H), 6.91-6.94 (d, J=8.1 Hz, 2H), 7.29 (s, 1H), 7.34-7.37 (d, J=8.1 Hz, 2H)

### EXAMPLE 38

### Preparation of 1-n-pentadecyl-2-(4-fluorophenyl)methyl-3,4-dihydro-6,7-dimethoxyisoquinolinium chloride (Compound No. 38)

Palmitoyl chloride, instead of caproyl chloride of Example 34, was treated by the same process described in Example 34, to give 1.07 g of oily compound, 1-n-pentadecyl-2-(2-fluorophenyl)methyl-3,4-dihydro-6,7-dimethoxyisoquinolinium chloride.

¹H-NMR (CDCl₃, 300 MHz): δ 0.88 (t, 3H), 1.24 (m, 22H), 1.48 (m, 2H), 1.71 (m, 3′ 2H), 3.15 (br t, 2H), 3.30 (m 2H), 3.82 (s, 3H), 3.95 (s, 3H), 4.00 (s, 3H), 4.13 (br t, 2H), 5.52 (s, 2H), 6.84 (s, 1H), 6.92-6.95 (d, J=8.8 Hz, 2H), 7.28 (s, 1H), 7.34-7.37 (d, J=8.8 Hz, 2H)

### EXAMPLE 39

### Preparation of 1-methyl-2-(3,4-difluorophenyl)methyl-3,4-dihydro-6,7-dimethoxyisoquinolinium chloride (Compound No. 39)

To a 250 ml of methanol solution containing 14.50 g of 3,4-dimethoxyphenethylamine was added 11.9 g of 3,4-difluorobenzaldehyde, and was heated for 2-3 under reflux and cooled to room temperature. Then, 3.03 g of sodium borohydride was added slowly to the reaction mixture obtained in the above in ice bath, and the resulted mixture was stirred for 1 hour at room temperature and methanol was removed from the reaction mixture under reduced pressure. The reaction mixture was suspended into 200 ml of dichloromethane and 200 ml of distilled water was added to the suspension. The phases were separated to obtain organic phase. The water phase was extracted twice with 200 ml of dichloromethane, and the organic phase thus separated was dried over MgSO₄, filtered and then concentrated under reduced pressure to prepare N-(3′,4′-difluorophenyl)methyl-3,4-dimethoxyphenethylamine, quantitatively.

1.23 G of the amine thus obtained was dissolved in 25 ml of 1,2-dichloroethane, and 0.56 ml of triethylamine was added thereto. Then, 0.26 ml of acetyl chloride was added slowly to the mixture at 0° C., and the reaction was proceeded at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 1.17 g of solid compound. 1-methyl-2-(3,4-difluorophenyl)methyl-3,4-dihydro-6,7-dimethoxyisoquinolinium chloride (m.p. 88° C.).

¹H-NMR (CDCl₃, 300 MHz): δ 3.03 (s, 3H), 3.14 (t, 2H), 3.97 (s, 3H), 4.01 (s, 3H), 4.04 (br t, 2H), 5.42 (s, 2H), 6.81 (s, 1H), 7.14-7.24 (m, 4H), 7.32 (s, 1H)

### EXAMPLE 40

### Preparation of 1-i-propyl-2-(3,4-difluorophenyl)methyl-3,4-dihydro-6,7-dimethoxyisoquinolinium chloride (Compound No. 40)

To a 25 ml of 1,2-dichloroethane solution containing 1.23 g of N-(3′,4′-difluorophenyl)methyl-3,4-dimethoxyphenethylamine was added 0.50 ml of triethylamine. Then, 0.46 ml of i-butyryl chloride was slowly added to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 0.97 g of oily compound, 1-i-propyl-2-(3,4-difluorophenyl)methyl-3,4-dihydro-6,7-dimethoxyisoquinolinium chloride.

¹H-NMR (CDCl₃, 300 MHz): δ 1.60 (s, 3H), 1.62 (s, 3H), 3.11 (br t, 2H), 3.39 (m, 1H), 3.93 (s, 3H), 4.01 (s, 3H), 4.12 (br t, 3H), 5.63 (s, 2H), 6.91 (s, 1H), 7.12-7.24 (m, 2H), 7.55-7.61 (m, 1H)

### EXAMPLE 41

### Preparation of 1-n-pentyl-2-(3,4-difluorophenyl)methyl-3,4-dihydro-6,7-dimethoxyisoquinolinium chloride (Compound No. 41)

Caproyl chloride, instead of isobutyryl chloride of Example 40, was treated by the same process described in Example 40, to give 1.10 g of oily compound, 1-n-pentyl-2-(3,4-difluorophenyl)methyl-3,4-dihydro-6,7-dimethoxyisoquinolinium chloride.

¹H-NMR (CDCl₃, 300 MHz): δ 0.88 (t, 3H), 1.25-1.48 (m, 4H), 1.69 (m, 2H), 3.20 (t, 2H), 3.38 (t, 2H), 3.97 (s, 3H), 4.02 (s, 3H), 4.15 (t, 2H), 5.68 (s, 2H), 6.93 (s, 1H), 7.16 (s, 1H), 7.22-7.30 (m, 2H), 7.40-7.46 (m, 1H)

### EXAMPLE 42

### Preparation of 1-n-hexyl-2-(3,4-difluorophenyl)methyl-3,4-dihydro-6,7-dimethoxyisoquinolinium chloride (Compound No. 42)

Heptanoyl chloride, instead of isobutyryl chloride of Example 40, was treated by the same process described in Example 40, to give 1.13 g of oily compound, 1-n-hexyl-2-(3,4-difluorophenyl)methyl-3,4-dihydro-6,7-dimethoxyisoquinolinium chloride.

¹H-NMR (CDCl₃, 300 MHz): δ 0.87 (t, 3H), 1.28 (m, 4H), 1.48 (m, 2H), 1.67 (m, 2H), 3.18 (br t, 2H), 3.32 (m, 2H), 3.96 (s, 3H), 4.02 (s, 3H), 4.12 (br t, 2H), 5.59 (s, 2H), 6.91 (s, 1H), 7.17-7.39 (m, 4H)

### EXAMPLE 43

### Preparation of 1-n-heptyl-2-(3,4-difluorophenyl)methyl-3,4-dihydro-6,7-dimethoxyisoquinolinium chloride (Compound No. 43)

Octanoyl chloride, instead of isobutyryl chloride of Example 40, was treated by the same process described in Example 40, to give 1.18 g of oily compound, 1-n-heptyl-2-(3,4-difluorophenyl)methyl-3,4-dihydro-6,7-dimethoxyisoquinolinium chloride.

¹H-NMR (CDCl₃, 300 MHz): δ 0.87 (t, 3H), 1.26 (m, 6H), 1.47 (m, 2H), 1.67 (m, 2H), 3.18 (m, 2H), 3.29 (m, 2H), 3.96 (s, 3H), 4.02 (s, 3H), 4.14 (m, 2H), 5.60 (s, 2H), 6.88 (s, 1H) 7.18-7.31 (m, 4H)

### EXAMPLE 44

### Preparation of 1-n-undecyl-2-(3,4-difluorophenyl)methyl-3,4-dihydro-6,7-dimethoxyisoquinolinium chloride (Compound No. 44)

Lauroyl chloride, instead of isobutyryl chloride of Example 40, was treated by the same process described in Example 40, to give 1.16 g of oily compound, 1-n-undecyl-2-(3,4-difluorophenyl)methyl-3,4-dihydro-6,7-dimethoxyisoquinolinium chloride.

¹H-NMR (CDCl₃, 300 MHz): δ 0.87 (t, 3H), 1.23 (m, 14H), 1.45 (m, 2H), 1.62 (m, 2H), 3.14 (br t, 2H), 3.24 (br t, 2H), 3.93 (s, 3H), 3.99 (s, 3H), 4.05 (br t, 2H), 5.43 (s, 2H), 6.87 (s, 1H), 7.12 (t, 1H), 7.22-7.27 (m, 4H)

### EXAMPLE 45

### Preparation of 1-n-hexyl-2-(4-trifluoromethylphenyl)methyl-3,4-dihydro-6,7-dimethoxyisoquinolinium chloride (Compound No. 45)

Heptanoyl chloride, instead of caproyl chloride of Example 27, was treated by the same process described in Example 40, to give 1.09 g of oily compound, 1-n-hexyl-2-(4-trifluoromethylphenyl)methyl-3,4-dihydro-6,7-dimethoxyisoquinolinium chloride.

¹H-NMR (CDCl₃, 300 MHz): δ 0.79 (t, 3H), 1.15-1.20 (m, 4H), 1.24 (m, 2H), 1.32 (m, 2H), 3.14 (br t, 2H), 3.24 (q, 2H), 3.90 (s, 3H), 3.95 (s, 3H), 4.12 (br t, 2H), 5.50 (s, 2H), 6.92 (s, 1H), 7.27 (s, 1H), 7.53 (d, J=9 Hz, 2H), 7.62 (d, J=9 Hz, 2H)

### EXAMPLE 46

### Preparation of 1-n-pentadecyl-2-(3,4-difluorophenyl)methyl-3,4-dihydro-6,7-dimethoxyisoquinolinium chloride (Compound No. 46)

Palmitoyl chloride, instead of isobutyryl chloride of Example 40, was treated by the same process described in Example 40, to give 1.07 g of oily compound, 1-n-pentadecyl-2-(3,4-difluorophenyl)methyl-3,4-dihydro-6,7-dimethoxyisoquinolinium chloride.

¹H-NMR (CDCl₃, 300 MHz): δ 0.88 (t, 3H), 1.25 (m, 22H), 1.49 (m, 2H), 1.66 (m, 2H), 3.20 (t, 2H), 3.34 (t, 2H), 3.96 (s, 3H), 4.02 (s, 3H), 4.18 (t, 2H), 5.73 (s, 2H), 6.92 (s, 1H), 7.18-7.27 (m, 1H), 7.29 (s, 1H), 7.32-7.42 (m, 2H)

### EXAMPLE 47

### Preparation of 1-(2-fluorophenyl)-2-(3,4-difluorophenyl)methyl-3,4-dihydro-6,7-dimethoxyisoquinolinium chloride (Compound No. 47)

2-Fluorobenzoyl chloride, instead of isobutyryl chloride of Example 40, was treated by the same process described in Example 40, to give 1.02 g of oily compound, 1-(2-fluorophenyl)-2-(3,4-difluorophenyl)methyl-3,4-dihydro-6,7-dimethoxyisoquinolinium chloride (m.p. 77˜79° C.).

¹H-NMR (CDCl₃, 300 MHz): d 3.10 (m, 2H), 3.62 (s, 3H), 3.89 (m, 2H), 4.02 (s, 3H), 5.12 (d, J=15 Hz, 1H), 5.60 (d, J=15 Hz, 1H), 6.42 (s, 1H), 6.86 (s, 1H), 7.15-7.23 (3H), 7.32-7.38 (t, 1H), 7.52-7.57 (t, 1H), 7.71-7.73 (q, 1H), 8.47 (t, 1H)

### EXAMPLE 48

### Preparation of 1-(4-t-butylphenyl)-2-(3,4-difluorophenyl)methyl-3,4-dihydro-6,7-dimethoxyisoquinolinium chloride (Compound No. 48)

4-t-Butylphenyl chloride, instead of isobutyryl chloride of Example 40, was treated by the same process described in Example 40, to give 1.41 g of solid compound, 1-(4-t-butylphenyl)-2-(3,4-difluorophenyl)methyl-3,4-dihydro-6,7-dimethoxyisoquinolinium chloride (m.p. 97° C.).

¹H-NMR (CDCl₃, 300 MHz): δ 1.39 (s, 9H), 3.20 (br t, 2H), 3.61 (s, 3H), 4.02 (s, 3H), 4.13 (br t, 2H), 5.13 (s, 2H), 6.44 (s, 1H), 6.88-6.92 (m, 1H), 6.96-7.05 (m, 1H), 7.10-7.20 (m, 2H), 7.61-7.70 (m, 4H)

### EXAMPLE 49

### Preparation of 1-methyl-2-(2-chlorophenyl)methyl-3,4-dihydro-6,7-dimethoxyisoquinolinium chloride (Compound No. 49)

To a 250 ml of methanol solution containing 14.50 g of 3,4-dimethoxyphenethylamine was added 11.8 g of 2-chlorobenzaldehyde and was heated for 2-3 under reflux and cooled to room temperature. Then, 3.03 g of sodium borohydride was added slowly to the reaction mixture obtained in the above in ice bath, and the resulted mixture was stirred for 1 hour at room temperature and methanol was removed from the reaction mixture under reduced pressure. The reaction mixture was suspended into 200 ml of dichloromethane and 200 ml of distilled water was added to the suspension. The phases were separated to obtain organic phase. The aqueous phase was extracted twice with 200 ml of dichloromethane, and the organic phase thus separated was dried over MgSO₄, filtered and then concentrated under reduced pressure to prepare N-(2′-chlorophenyl)methyl-3,4-dimethoxyphenethylamine, quantitatively.

1.22 G of the amine thus obtained was dissolved in 25 ml of 1,2-dichloroethane, and 0.56 ml of triethylamine was added thereto. Then, 0.26 ml of acetyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 0.98 g of oily compound, 1-methyl-2-(2-chlorophenyl)methyl-3,4-dihydro-6,7-dimethoxyisoquinolinium chloride.

¹H-NMR (CDCl₃, 300 MHz): δ 3.03 (s, 3H), 3.11 (br t, 2H), 3.83 (br t, 2H), 3.98 (s, 6H), 5.59 (s, 2H), 6.79 (s, 1H), 7.28-7.45 (m, 4H), 7.78 (m, 1H)

### EXAMPLE 50

### Preparation of 1-n-pentyl-2-(2-chlorophenyl)methyl-3,4-dihydro-6,7-dimethoxyisoquinolinium chloride (Compound No. 50)

To a 25 ml of 1,2-dichloroethane solution containing 1.22 g of N-(2′-chlorophenyl)methyl-3,4-dimethoxyphenethylamine was added 0.50 ml of triethylamine. Then, 0.96 ml of caproyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1) as an elute solvent, to give 1.10 g of oily compound, 1-n-pentyl-2-(2-chlorophenyl)methyl-3,4-dihydro-6,7-dimethoxyisoquinolinium chloride.

¹H-NMR (CDCl₃, 300 MHz): δ 0.87 (t, 3H), 1.31-1.45 (m, 4H), 1.66 (m, 2H), 3.21 (t, 2H), 3.37 (t, 2H), 3.96 (s, 3H), 4.00 (s, 3H), 4.02 (t, 2H), 5.60 (s, 2H), 6.96 (s, 1H), 7.31-7.44 (m, 4H), 7.77 (m, 1H)

### EXAMPLE 51

### Preparation of 1-n-hexyl-2-(2-chlorophenyl)methyl-3,4-dihydro-6,7-dimethoxyisoquinolinium chloride (Compound No. 51)

Heptanoyl chloride, instead of caproyl chloride of Example 50, was treated by the same process described in Example 50, to give 1.13 g of solid compound, 1-n-hexyl-2-(2-chlorophenyl)methyl-3,4-dihydro-6,7-dimethoxyisoquinolinium chloride.

¹H-NMR (CDCl₃, 300 MHz): δ 0.86 (t, 3H), 1.28 (m, 4H), 1.46 (m, 2H), 1.64 (m, 2H), 3.20 (br t, 2H), 3.29 (m, 2H), 3.95 (s, 3H), 4.00 (s, 3H), 4.12 (br t, 2H), 5.54 (s, 2H), 6.94 (s, 1H), 7.28 (s, 1H), 7.36-7.44 (m, 3H), 7.73-7.75 (m, 1H)

### EXAMPLE 52

### Preparation of 1-n-heptyl-2-(2-chlorophenyl)methyl-3,4-dihydro-6,7-dimethoxyisoquinolinium chloride (Compound No. 52)

Octanoyl chloride, instead of caproyl chloride of Example 50, was treated by the same process described in Example 50, to give 1.07 g of solid compound, 1-n-heptyl-2-(2-chlorophenyl)methyl-3,4-dihydro-6,7-dimethoxyisoquinolinium chloride.

¹H-NMR (CDCl₃, 300 MHz): δ 0.88 (t, 3H), 1.21-1.28 (m, 6H), 1.50 (m, 2H), 1.75 (m, 2H), 3.19 (br t, 2H), 3.34 (m, 2H), 3.96 (s, 3H), 4.00 (s, 3H), 4.02 (br t, 2H), 5.77 (s, 2H), 6.81 (s, 1H), 7.25 (s, 1H), 7.41-7.43 (m, 3H), 8.00 (m, 1H)

### EXAMPLE 53

### Preparation of 1-n-undecyl-2-(2-chlorophenyl)methyl-3,4-dihydro-6,7-dimethoxyisoquinolinium chloride (Compound No. 53)

Lauroyl chloride, instead of caproyl chloride of Example 50, was treated by the same process described in Example 50, to give 1.16 g of oily compound, 1-n-undecyl-2-(2-chlorophenyl)methyl-3,4-dihydro-6,7-dimethoxy-isoquinolinium chloride.

¹H-NMR (CDCl₃, 300 MHz): δ 0.87 (t, 3H), 1.24 (m, 14H), 1.47 (m, 2H), 1.68 (m, 2H), 3.19 (br t, 2H), 3.30 (br t, 2H), 3.95 (s, 3H), 4.01 (s, 3H), 4.03 (br t, 2H), 5.53 (s, 2H), 6.92 (s, 1H), 7.29 (s, 1H), 7.40-7.46 (m, 3H), 7.72 (m, 1H)

### EXAMPLE 54

### Preparation of 1-n-pentadecyl-2-(2-chlorophenyl)methyl-3,4-dihydro-6,7-dimethoxyisoquinolinium chloride (Compound No. 54)

Palmitoyl chloride, instead of caproyl chloride of Example 50, was treated by the same process described in Example 50, to give 1.07 g of oily compound, 1-n-pentadecyl-2-(2-chlorophenyl)methyl-3,4-dihydro-6,7-dimethoxyisoquinolinium chloride.

¹H-NMR (CDCl₃, 300 MHz): δ 0.88 (t, 3H), 1.25 (m, 22H), 1.48 (m, 2H), 1.73 (m, 2H), 3.16 (br t, 2H), 3.30 (m 2H), 3.95 (s, 3H), 4.01 (s, 3H), 4.05 (br t, 2H), 5.54 (s, 2H), 6.84 (s, 1H), 7.27 (s, 1H), 7.41-7.47 (m, 3H), 7.72-7.78 (m. 1H)

### EXAMPLE 55

### Preparation of 1-(4-t-butylphenyl)-2-(2-chlorophenyl)methyl-3,4-dihydro-6,7-dimethoxyisoquinolinium chloride (Compound No. 55)

4-t-Butylbenzoyl chloride, instead of caproyl chloride of Example 50, was treated by the same process described in Example 50, to give 1.21 g of solid compound, 1-(4-t-butylphenyl)-2-(2-chlorophenyl)methyl-3,4-dihydro-6,7-dimethoxyisoquinolinium chloride (m.p. 117° C.).

¹H-NMR (CDCl₃, 300 MHz): δ 1.39 (s, 9H), 3.09 (br t, 2H), 3.62 (s, 3H), 4.01 (s, 3H), 4.29 (br t, 2H), 5.57 (s, 2H), 6.49 (s, 1H), 6.85 (s, 1H), 7.35 (m, 3H), 7.65 (d, 2H), 7.78-7.80 (m, 3H)

### EXAMPLE 56

### Preparation of 1-methyl-2-(2,6-difluorophenyl)methyl-3,4-dihydro-6,7-dimethoxyisoquinolinium chloride (Compound No. 56)

To a 250 ml of methanol solution containing 14.50 g of 3,4-dimethoxyphenethylamine was added 11.9 g of 2,6-difluorobenzaldehyde and was heated for 2-3 under reflux and cooled to room temperature. Then, 3.03 g of sodium borohydride was added slowly to the reaction mixture obtained in the above in ice bath, and the resulted mixture was stirred for 1 hour at room temperature and methanol was removed from the reaction mixture under reduced pressure. The reaction mixture was suspended into 200 ml of dichloromethane and 200 ml of distilled water was added to the suspension. The phases were separated to obtain organic phase. The aqueous phase was extracted twice with 200 ml of dichloromethane, and the organic phase thus separated was dried over MgSO₄, filtered and then concentrated under reduced pressure to prepare N-(3′,4′-difluorophenyl)methyl-3,4-dimethoxyphenethylamine, quantitatively.

To a 25 ml of 1,2-dichloroethane solution containing 1.23 g of the amine thus obtained was added 0.56 ml of triethylamine. Then, 0.26 ml of acetyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 1.12 g of oily compound, 1-methyl-2-(2,6-difluorophenyl)methyl-3,4-dihydro-6,7-dimethoxyisoquinolinium chloride.

¹H-NMR (CDCl₃, 300 MHz): δ 3.00 (s, 3H), 3.02 (br t, 2H), 3.84 (t, 2H), 3.87 (s, 3H), 3.90 (s, 3H), 5.41 (s, 2H), 7.13 (s, 1H), 7.27 (t, 2H), 7.56 (s, 1H), 7.57-7.65 (m, 1H)

### EXAMPLE 57

### Preparation of 1-n-pentyl-2-(2,6-difluorophenyl)methyl-3,4-dihydro-6,7-dimethoxyisoquinolinium chloride (Compound No. 57)

To a 25 ml of 1,2-dichloroethane solution containing 1.23 g of N-(2,6-difluorophenyl)methyl-3,4-dimethoxyphenethylamine was added 0.50 ml of tri-ethylamine. Then, 0.96 ml of caproyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1) as an elute solvent, to give 1.10 g of solid compound, 1-n-pentyl-2-(2,6-difluorophenyl)methyl-3,4-dihydro-6,7-dimethoxyisoquinolinium chloride (m.p. 196° C.).

¹H-NMR (CDCl₃, 300 MHz): δ 0.87 (t, 3H), 1.27-1.36 (m, 2H), 1.40-1.45 (m, 2H), 1.50-1.56 (m, 2H), 3.22 (t, 2H), 3.36 (t, 2H), 3.94 (s, 3H), 4.01 (s, 3H), 4.24 (t, 2H), 5.60 (s, 2H), 6.91 (s, 1H), 7.01-7.06 (m 2H), 7.29 (s, 1H), 7.42-7.47 (m, 1H)

### EXAMPLE 58

### Preparation of 1-n-hexyl-2-(2,6-difluorophenyl)methyl-3,4-dihydro-6,7-dimethoxyisoquinolinium chloride (Compound No. 58)

Heptanoyl chloride, instead of caproyl chloride of Example 57, was treated by the same process described in Example 57, to give 1.13 g of solid compound, 1-n-hexyl-2-(2,6-difluorophenyl)methyl-3,4-dihydro-6,7-dimethoxy-isoquinolinium chloride (m.p. 198˜199° C.).

¹H-NMR (CDCl₃, 300 MHz): δ 0.87 (t, 3H), 1.27 (m, 4H), 1.44 (m, 2H), 1.54 (m, 2H), 3.22 (t, 2H), 3.34 (t, 2H), 3.94 (s, 3H), 4.01 (s, 3H), 4.22 (t, 2H), 5.59 (s, 2H), 6.94 (s, 1H), 7.01-7.07 (m, 2H), 7.29 (s, 1H), 7.40-7.51 (m, 1H)

### EXAMPLE 59

### Preparation of 1-n-undecyl-2-(2,6-difluorophenyl)methyl-3,4-dihydro-6,7-dimethoxyisoquinolinium chloride (Compound No. 59)

Lauroyl chloride, instead of caproyl chloride of Example 57, was treated by the same process described in Example 57, to give 1.16 g of oily compound, 1-n-undecyl-2-(2,6-difluorophenyl)methyl-3,4-dihydro-6,7-dimethoxyisoquinolinium chloride.

¹H-NMR (CDCl₃, 300 MHz): δ 0.88 (t, 3H), 1.24 (m, 14H), 1.43 (m, 2H), 1.51 (m, 2H), 3.20 (br t, 2H), 3.32 (br t, 2H), 3.94 (s, 3H), 4.01 (s, 3H), 4.27 (br t, 2H), 5.56 (s, 2H), 6.91 (s, 1H), 7.03 (t, 2H), 7.27 (s, 2H), 7.42-7.47 (m, 1H)

### EXAMPLE 60

### Preparation of 1-n-pentadecyl-2-(2,6-difluorophenyl)methyl-3,4-dihydro-6,7-dimethoxyisoquinolinium chloride (Compound No. 60)

Palmitoyl chloride, instead of caproyl chloride of Example 57, was treated by the same process described in Example 57, to give 1.07 g of solid compound, 1-n-pentadecyl-2-(2,6-difluorophenyl)methyl-3,4-dihydro-6,7-dimethoxyisoquinolinium chloride (m.p. 104° C.).

¹H-NMR (CDCl₃, 300 MHz): δ 0.88 (t, 3H), 1.24 (m, 22H), 1.43 (m, 2H), 1.52 (m, 2H), 3.22 (br t, 2H), 3.34 (m 2H), 3.94 (s, 3H), 4.02 (s, 3H), 4.22 (br t, 2H), 5.57 (s, 2H), 6.97 (s, 1H), 7.01-7.07 (m, 2H), 7.29 (s, 1H), 7.41-7.48 (m. 1H)

### EXAMPLE 61

### Preparation of 1-n-heptyl-2-(2,6-difluorophenyl)methyl-3,4-dihydro-6,7-dimethoxyisoquinolinium chloride (Compound No. 61)

Octanoyl chloride, instead of caproyl chloride of Example 57, was treated by the same process described in Example 57, to give 1.18 g of oily compound, 1-n-heptyl-2-(2,6-difluorophenyl)methyl-3,4-dihydro-6,7-dimethoxy-isoquinolinium chloride.

¹H-NMR (CDCl₃, 300 MHz): δ 0.85 (t, 3H), 1.20 (m, 6H), 1.39 (m, 4H), 3.18 (br t, 2H), 3.30 (m, 2H), 3.93 (s, 3H), 3.99 (s, 3H), 4.20 (m, 2H), 5.48 (s, 2H), 6.97 (s, 1H), 6.98-7.04 (m, 2H), 7.28 (d, 1H), 7.40-7.44 (m, 1H)

### EXAMPLE 62

### Preparation of 1-(2-fluorophenyl)-2-(2,6-difluorophenyl)methyl-3,4-dihydro-6,7-dimethoxyisoquinolinium chloride (Compound No. 62)

2-Fluorobenzoyl chloride, instead of caproyl chloride of Example 57, was treated by the same process described in Example 57, to give 0.94 g of solid compound, 1-(2-fluorophenyl)-2-(2,6-difluorophenyl)methyl-3,4-dihydro-6,7-dimethoxyisoquinolinium chloride (m.p. 79° C.).

¹H-NMR (CDCl₃, 300 MHz): δ 3.29 (m, 2H), 3.61 (s, 3H), 4.04 (s, 6H), 4.12-4.14 (m, 1H), 4.63-4.82 (m, 1H), 5.27 (d, J=15 Hz, 1H), 5.37 (d, J=15 Hz, 1H), 6.42 (s, 1H), 6.92-7.00 (m, 3H), 7.27 (t, 1H), 7.37-7.40 (m, 1H), 7.54 (t, 1H), 7.70-7.72 (m, 1H), 8.32 (t, 1H)

### EXAMPLE 63

### Preparation of 1-(4-t-butylphenyl)-2-(2,6-difluorophenyl)methyl-3,4-dihydro-6,7-dimethoxyisoquinolinium chloride (Compound No. 63)

4-t-Butylbenzoyl chloride, instead of caproyl chloride of Example 57, was treated by the same process described in Example 57, to give 1.23 g of solid compound, 1-(4-t-butylphenyl)-2-(2,6-difluorophenyl)methyl-3,4-dihydro-6,7-dimethoxyisoquinolinium chloride (m.p. 85˜87° C.).

¹H-NMR (CDCl₃, 300 MHz): δ 1.37 (s, 9H), 3.24 (t, 2H), 3.55 (s, 6H), 4.00 (s, 3H), 4.33 (t, 2H), 5.29 (s, 2H), 6.39 (s, 1H), 6.89 (t, 2H), 7.10 (s, 1H), 7.27-7.36 (m, 1H), 7.62 (m, 4H)

### EXAMPLE 64

### Preparation of 1-methyl-2-(2-chloro-6-fluorophenyl)methyl-3,4-dihydro-6,7-dimethoxyisoquinolinium chloride (Compound No. 64)

To a 250 ml of methanol solution containing 14.50 g of 3,4-dimethoxyphenethylamine was added 13.49 g of 2-chloro-6-fluorobenzaldehyde and was heated for 2-3 under reflux and cooled to room temperature. Then, 3.03 g of sodium borohydride was added slowly to the reaction mixture obtained in the above in ice bath, and the resulted mixture was stirred for 1 hour at room temperature and methanol was removed from the reaction mixture under reduced pressure. The reaction mixture was suspended into 200 ml of dichloromethane and 200 ml of distilled water was added to the suspension. The phases were separated to obtain organic phase. The aqueous phase was extracted twice with 200 ml of dichloromethane, and the organic phase thus separated was dried over MgSO₄, filtered and then concentrated under reduced pressure to prepare N-(2-chloro-6-fluorophenyl)methyl-3,4-dimethoxyphenethylamine, quantitatively.

To a 25 ml of 1,2-dichloroethane solution containing 1.30 g of the amine thus obtained was added 0.56 ml of triethylamine. Then, 0.26 ml of acetyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 1.06 g of oily compound, 1-methyl-2-(2-chloro-6-fluorophenyl)methyl-3,4-dihydro-6,7-dimethoxyisoquinolinium chloride.

¹H-NMR (CDCl₃, 300 MHz): δ 3.10 (br t, 2H), 3.17 (s, 3H), 3.88 (br t, 2H), 3.99 (s, 6H), 5.55 (s, 2H), 6.85 (s, 1H), 7.13 (s, 1H), 7.31-7.34 (d, 1H), 7.39-7.46 (m, 2H)

### EXAMPLE 65

### Preparation of 1-i-propyl-2-(2-chloro-6-fluorophenyl)methyl-3,4-dihydro-6,7-dimethoxyisoquinolinium chloride (Compound No. 65)

To a 25 ml of 1,2-dichloroethane solution containing 1.30 g of N-(2-chloro-6-fluorophenyl)methyl-3,4-dimethoxyphenethylamine was added 0.50 ml of triethylamine. Then, 0.46 ml of i-butyryl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 0.89 g of solid compound, 1-i-propyl-2-(2-chloro-6-fluorophenyl)methyl-3,4-dihydro-6,7-dimethoxyisoquinolinium chloride (m.p. 116˜118° C.).

¹H-NMR (CDCl₃, 300 MHz): δ 1.60 (s, 3H), 1.62 (s, 3H), 3.10 (br t, 2H), 3.93 (s, 3H), 3.96 (br s, 3H), 4.01 (s, 3H), 5.64 (s, 2H), 6.93 (s, 1H), 7.13 (t, 1H), 7.36-7.44 (m, 3H)

### EXAMPLE 66

### Preparation of 1-n-pentyl-2-(2-chloro-6-fluorophenyl)methyl-3,4-dihydro-6,7-dimethoxyisoquinolinium chloride (Compound No. 66)

Caproyl chloride, instead of i-butyryl chloride of Example 65, was treated by the same process described in Example 65, to give 1.10 g of oily compound, 1-n-pentyl-2-(2-chloro-6-fluorophenyl)methyl-3,4-dihydro-6,7-dimethoxyisoquinolinium chloride.

¹H-NMR (CDCl₃, 300 MHz): δ 0.88 (t, 3H), 1.25-1.47 (m, 4H), 1.60 (m, 2H), 3.21 (t, 2H), 3.37 (t, 2H), 3.96 (s, 3H), 4.02 (s, 3H), 4.06 (t, 2H), 5.64 (s, 2H), 7.02 (s, 1H), 7.16 (t 1H), 7.34-7.37 (m, 2H), 7.42-7.49 (m, 1H)

### EXAMPLE 67

### Preparation of 1-n-heptyl-2-(2-chloro-6-fluorophenyl)methyl-3,4-dihydro-6,7-dimethoxyisoquinolinium chloride (Compound No. 67)

Octanoyl chloride, instead of i-butyryl chloride of Example 65, was treated by the same process described in Example 65, to give 0.91 g of solid compound, 1-n-heptyl-2-(2-chloro-6-fluorophenyl)methyl-3,4-dihydro-6,7-dimethoxyisoquinolinium chloride (m.p. 170˜172° C.).

¹H-NMR (CDCl₃, 300 MHz): δ 0.85 (t, 3H), 1.23 (m, 6H), 1.40 (m, 2H), 1.52 (m, 2H), 3.02 (t, 2H), 3.36 (m, 2H), 3.74 (t, 2H), 3.87 (s, 3H), 3.92 (s, 3H), 5.57 (s, 2H), 7.15 (s, 1H), 7.42 (t, 1H), 7.51-7.60 (m, 2H)

### EXAMPLE 68

### Preparation of 1-n-undecyl-2-(2-chloro-6-fluorophenyl)methyl-3,4-dihydro-6,7-dimethoxyisoquinolinium chloride (Compound No. 68)

Lauroyl chloride, instead of i-butyryl chloride of Example 65, was treated by the same process described in Example 65, to give 1.16 g of oily compound, 1-n-undecyl-2-(2-chloro-6-fluorophenyl)methyl-3,4-dihydro-6,7-dimethoxyisoquinolinium chloride.

¹H-NMR (CDCl₃, 300 MHz): δ 0.88 (t, 3H), 1.24 (m, 14H), 1.43 (m, 2H), 1.56 (m, 2H), 3.20 (br t, 2H), 3.33 (br t, 2H), 3.94 (s, 3H), 4.01 (s, 3H), 4.10 (br t, 2H), 5.66 (s, 2H), 6.94 (s, 1H), 7.13 (t, 1H), 7.29-7.43 (m, 4H)

### EXAMPLE 69

### Preparation of 1-(2-fluorophenyl)-2-(2-chloro-6-fluorophenyl)methyl-3,4-dihydro-6,7-dimethoxyisoquinolinium chloride (Compound No. 69)

2-Fluorobenzoyl chloride, instead of i-butyryl chloride of Example 65, was treated by the same process described in Example 65, to give 0.92 g of solid compound, 1-(2-fluorophenyl)-2-(2-chloro-6-fluorophenyl)methyl-3,4-dihydro-6,7-dimethoxyisoquinolinium chloride (m.p. 140˜142° C.).

¹H-NMR (CDCl₃, 300 MHz): δ 3.23 (t, 2H), 3.63 (s, 3H), 3.99 (br t, 2H), 4.05 (s, 3H), 5.26 (d, J=15 Hz, 1H), 5.47 (d, J=15 Hz, 1H), 6.43 (s, 1H), 7.04 (s, 1H), 7.08 (t, 1H), 7.29 (t, 1H), 7.34-7.40 (m, 2H), 7.55 (t, 1H), 7.68-7.79 (m, 1H), 8.16 (t, 1H)

### EXAMPLE 70

### Preparation of 1-(4-t-butylphenyl)-2-(2-chloro-6-fluorophenyl)methyl-3,4-dihydro-6,7-dimethoxyisoquinolinium chloride (Compound No. 70)

4-t-Butylbenzoyl chloride, instead of i-butyryl chloride of Example 65, was treated by the same process described in Example 65, to give 1.21 g of solid compound, 1-(4-t-butylphenyl)-2-(2-chloro-6-fluorophenyl)methyl-3,4-dihydro-6,7-dimethoxyisoquinolinium chloride (m.p. 98˜100° C.).

¹H-NMR (CDCl₃, 300 MHz): δ 1.40 (s, 9H), 3.12 (t, 2H), 3.63 (s, 3H), 4.04 (s, 3H), 4.19 (t, 2H), 5.35 (s, 2H), 6.47 (s, 1H, 6.98 (s, 1H), 7.06 (t, 1H), 7.26-7.28 (m, 1H), 7.31-7.40 (m, 1H), 7.58-7.64 (m, 4H)

### EXAMPLE 71

### Preparation of 1-methyl-2-(2-nitrophenyl)methyl-3,4-dihydro-6,7-dimethoxyisoquinolinium chloride (Compound No. 71)

To a 250 ml of methanol solution containing 14.50 g of 3,4-dimethoxyphenethylamine was added 12.69 g of 2-nitrobenzaldehyde and was heated for 2-3 under reflux and cooled to room temperature. Then, 3.03 g of sodium borohydride was added slowly to the reaction mixture obtained in the above in ice bath, and the resulted mixture was stirred for 1 hour at room temperature and methanol was removed from the reaction mixture under reduced pressure. The reaction mixture was suspended into 200 ml of dichloromethane and 200 ml of distilled water was added to the suspension. The phases were separated to obtain organic phase. The aqueous phase was extracted twice with 200 ml of dichloromethane, and the organic phase thus separated was dried over MgSO₄, filtered and then concentrated under reduced pressure to prepare N-(2′-nitrophenyl)methyl-3,4-dimethoxyphenethylamine, quantitatively.

To a 25 ml of 1,2-dichloroethane solution containing 1.30 g of the amine thus obtained was added 0.56 ml of triethylamine. Then, 0.26 ml of acetyl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 0.92 g of solid compound, 1-methyl-2-(2-nitrophenyl)methyl-3,4-dihydro-6,7-dimethoxyisoquinolinium chloride (m.p. 130˜132° C.).

¹H-NMR (CDCl₃, 300 MHz): δ 2.88 (s, 3H), 3.13 (t, 2H), 3.89 (s, 3H), 3.93 (s, 3H), 3.96 (t, 2H), 5.67 (s, 2H), 7.19 (s, 1H), 7.60 (d, 2H), 7.71 (t, 1H), 7.83 (t, 1H), 8.28 (d, 1H)

### EXAMPLE 72

### Preparation of 1-i-propyl-2-(2-nitrophenyl)methyl-3,4-dihydro-6,7-dimethoxyisoquinolinium chloride (Compound No. 72)

To a 25 ml of 1,2-dichloroethane solution containing 1.27 g of N-(2′-nitrophenyl)methyl-3,4-dimethoxyphenethylamine was added 0.50 ml of tri-ethylamine. Then, 0.46 ml of i-butyryl chloride was added slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.56 ml of phosphoryl chloride was added thereto, and the reaction mixture was heated for 8 hours under reflux and cooled to room temperature. The solvent was concentrated under reduced pressure, and the reaction mixture was separated through silica-gel column chromatography eluting with dichloromethane and methanol (10:1), to give 0.81 g of solid compound, 1-i-propyl-2-(2-nitrophenyl)methyl-3,4-dihydro-6,7-dimethoxyisoquinolinium chloride (m.p. 138˜140° C.).

¹H-NMR (CDCl₃, 300 MHz): δ 1.46 (s, 3H), 1.48 (s, 3H), 3.14 (br t, 2H), 3.60 (m, 1H), 3.68 (br t, 2H), 3.88 (s, 3H), 3.84 (s, 3H), 5.67 (s, 2H), 7.25 (s, 1H), 7.47 (s, 1H), 7.58-7.64 (m, 1H), 7.66-7.73 (m, 1H), 7.78-7.86 (m, 1H), 8.26-8.33 (m, 1H)

### EXAMPLE 73

### Preparation of 1-n-heptyl-2-(2-nitrophenyl)methyl-3,4-dihydro-6,7-dimethoxyisoquinolinium chloride (Compound No. 73)

Octanoyl chloride, instead of i-butyryl chloride of Example 72, was treated by the same process described in Example 72, to give 0.80 g of solid compound, 1-n-heptyl-2-(2-nitrophenyl)methyl-3,4-dihydro-6,7-dimethoxyiso-quinolinium chloride (m.p. 145° C.).

¹H-NMR (CDCl₃, 300 MHz): δ 0.88 (t, 3H), 1.27-1.21 (m, 6H), 1.24 (m, 2H), 1.78 (m, 2H), 3.16 (t, 2H), 3.33 (m, 2H), 3.89 (t, 2H), 3.96 (s, 3H), 4.00 (s, 3H), 6.07 (s, 2H), 6.81 (s, 1H), 7.26 (s, 1H), 7.68 (t, 1H), 7.87 (t, 1H), 8.18 (d, 1H), 8.48 (d, 1H)

### EXAMPLE 74

### Preparation of 1-n-undecyl-2-(2-nitrophenyl)methyl-3,4-dihydro-6,7-dimethoxyisoquinolinium chloride (Compound No. 74)

Lauroyl chloride, instead of i-butyryl chloride of Example 72, was treated by the same process described in Example 72, to give 1.16 g of oily compound, 1-n-undecyl-2-(2-nitrophenyl)methyl-3,4-dihydro-6,7-dimethoxyisoquinolinium chloride.

¹H-NMR (CDCl₃, 300 MHz): δ 0.87 (t, 3H), 1.25 (m, 14H), 1.49 (m, 2H), 1.76 (m, 2H), 3.17 (br t, 2H), 3.34 (br t, 2H), 3.87 (br t, 2H), 3.93 (s, 3H), 4.00 (s, 3H), 6.07 (s, 2H), 6.83 (s, 1H), 7.29 (s, 1H), 7.67 (t, 1H), 7.86 (dt, J=1.2 Hz, 1H), 8.17-8.19 (dd, J=1.2 Hz, 1H), 8.41-8.44 (d, 1H)

### EXAMPLE 75

### Preparation of 1-(4-t-butylphenyl)-2-(2-nitrophenyl)methyl-3,4-dihydro-6,7-dimethoxyisoquinolinium chloride (Compound No. 75)

4-t-Butylbenzoyl chloride, instead of i-butyryl chloride of Example 72, was treated by the same process described in Example 72, to give 1.02 g of solid compound, 1-(4-t-butylphenyl)-2-(2-nitrophenyl)methyl-3,4-dihydro-6,7-dimethoxyisoquinolinium chloride (m.p. 78˜80° C.).

¹H-NMR (CDCl₃, 300 MHz): δ 1.38 (s, 9H), 3.11 (t, 2H), 3.63 (s, 3H), 4.02 (s, 3H), 4.12 (t, 2H), 5.86 (s, 2H), 6.50 (s, 1H), 6.85 (s, 1H), 7.61-7.66 (m, 3H), 7.79-7.82 (m, 3H), 8.12 (d, 1H), 8.24 (d, 1H)

### EXAMPLE 76

### Preparation of 1-n-heptyl-2-(4-hydroxy-3-nitrophenyl)methyl-3,4-dihydro-6,7-dimethoxyisoquinolinium chloride (Compound No. 76)

4-Hydroxy-3-nitrobenzaldehyde, instead of 2-nitrobenzaldehyde of Example 71, was treated by the same process described in Example 71, to give N-(4-hydroxy-3-nitrophenyl)methyl-3,4-dimethoxyphenethylamine. Then, the amine was treated except employing octanoyl chloride instead of acetyl chloride by the same process described in Example 71, to give 0.94 g of 1-n-heptyl-2-(4-hydroxy-3-nitrophenyl)methyl-3,4-dihydro-6,7-dimethoxyisoquinolinium chloride (m.p. 189˜190° C.).

¹H-NMR (CDCl₃, 300 MHz): δ 0.80 (t, 3H), 1.15-1.21 (m, 6H), 1.28 (m, 2H), 1.41 (m, 2H), 3.16 (t, 2H), 3.87 (s, 3H), 3.93 (s, 3H), 3.97 (t, 2H), 5.40 (s, 2H), 6.99 (d, 1H), 7.20 (s, 1H), 7.34 (s, 1H), 7.52 (s, 1H), 7.92 (d, 1H)

### EXAMPLE 77

### Preparation of 1-n-undecyl-2-(4-hydroxy-3-nitrophenyl)methyl-3,4-dihydro-6,7-dimethoxyisoquinolinium chloride (Compound No. 77)

Lauroyl chloride, instead of octanoyl chloride of Example 72, was treated by the same process described in Example 72, to give 1.16 g of oily compound, 1-n-undecyl-2-(4-hydroxy-3-nitrophenyl)methyl-3,4-dihydro-6,7-dimethoxyisoquinolinium chloride.

¹H-NMR (CDCl₃, 300 MHz): δ 0.88 (t, 3H), 1.22 (m, 14H), 1.45 (m, 2H), 1.67 (m, 2H), 3.28 (br t, 2H), 3.64 (br t, 2H), 3.95 (s, 3H), 4.02 (s, 3H), 4.20 (br t, 2H), 5.02 (br s, 1H), 5.68 (s, 2H), 6.91 (s, 1H), 7.09 (m, 1H), 7.29 (m, 1H), 7.41 (m, 1H), 8.00 (m, 1H)

### EXAMPLE 78

### Preparation of 1-methyl-2-(3-bromo-4,5-dimethoxy)methyl-3,4-dihydro-6,7-dimethoxyisoquinolinium chloride (Compound No. 78)

3-bromo-4,5-dimethoxybenzaldehyde, instead of 2-nitrobenzaldehyde of Example 71, was treated by the same process described in Example 71, to give N-(3-bromo-4,5-dimethoxyphenyl)methyl-3,4-dimethoxyphenethylamine. Then, the amine was treated employing acetyl chloride by the same process described in Example 71, to give 0.63 g of oily compound, 1-methyl-2-(3-bromo-4,5-dimethoxyphenyl)methyl-3,4-dihydro-6,7-dimethoxyisoquinolinium chloride.

¹H-NMR (CDCl₃, 300 MHz): δ 3.03 (s, 3H), 3.10 (br t, 2H), 3.80 (s, 3H), 3.89 (s, 3H), 3.95 (s, 3H), 3.96 (s, 3H), 4.03 (br t, 2H), 5.32 (s, 2H), 6.89 (s, 1H), 6.98 (s, 1H), 7.15 (s, 1H), 7.38 (s, 1H)

### EXAMPLE 79

### Preparation of 1-n-heptyl-2-(3-bromo-4,5-dimethoxyphenyl)methyl-3,4-dihydro-6,7-dimethoxyisoquinolinium chloride (Compound No. 79)

Octanoyl chloride, instead of acetyl chloride of Example 78, was treated by the same process described in Example 78, to give 0.75 g of oily compound, 1-n-heptyl-2-(3-bromo-4,5-dimethoxyphenyl)methyl-3,4-dihydro-6,7-dimethoxyisoquinolinium chloride.

¹H-NMR (CDCl₃, 300 MHz): δ 0.87 (t, 3H), 1.27 (m, 6H), 1.48 (m 2H), 1.64 (m, 2H), 3.17 (br t, 2H), 3.30 (m, 2H), 3.44 (s, 3H), 3.86 (s, 3H), 3.95 (s, 3H), 3.96 (s, 3H), 4.16 (br t, 2H), 5.57 (s, 2H), 6.87 (s, 1H), 6.91 (s, 1H), 7.28 (s, 1H), 7.37 (s, 1H)

### EXAMPLE 80

### Preparation of 1-methyl-2-(2-fluorophenyl)methyl-3,4-dihydro-6-methoxyisoquinolinium chloride (Compound No. 80)

9.97 G of 3-methoxyphenethylamine, instead of 3,4-dimethoxyphenethylamine of Example 1, was treated by the same process described in Example 1, to synthesize 0.82 g of N-(2-fluorophenyl)methyl-3-methoxyphenethylamine. The resulting mixture was dissolved in 25 ml of 1,2-dichloroethane and 0.50 ml of triethylamine was added thereto. Then, 0.26 ml of acetyl chloride was added dropwise slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄ and filtered and concentrated under reduced pressure to synthesize amide intermediate.

The crude intermediate thus obtained was dissolved in 25 ml of acetonitrile and 0.46 ml of phosphoryl chloride was added thereto, and the reaction mixture was heated for 8 hours under reflux and cooled to room temperature. The solvent was removed under reduced pressure, and the reaction mixture was separated through silica-gel column chromatography eluting with dichloromethane and methanol (10:1), to give 0.78 g of oily compound, 1-methyl-2-(2-fluorophenyl)methyl-3,4-dihydro-6-methoxyisoquinolinium chloride.

¹H-NMR (CDCl₃, 300 MHz): δ 3.08 (s, 3H), 3.08 (m, 2H), 3.93 (s, 3H), 4.11 (m, 2H), 5.72 (s, 2H), 6.81 (d, 1H, J=2.0 Hz), 6.96 (dd, 1H, J=2.0, 8.4 Hz), 7.11 (m, 1H), 7.26 (m, 1H), 7.44 (m, 1H), 7.86 (m, 2H)

### EXAMPLE 81

### Preparation of 1-ethyl-2-(2-fluorophenyl)methyl-3,4-dihydro-6-methoxyisoquinolinium chloride (Compound No. 81)

Propionyl chloride, instead of acetyl chloride of Example 80, was treated by the same process described in Example 80, to give 0.96 g of oily compound, 1-ethyl-2-(2-fluorophenyl)methyl-3,4-dihydro-6-methoxyisoquinolinium chloride.

¹H-NMR (CDCl₃, 300 MHz): δ 1.37 (t, 3H), 3.13 (t, 2H), 3.40 (q, 2H), 3.95 (s, 3H), 4.15 (t, 2H), 5.69 (s, 2H), 6.95 (m, 2H), 7.09 (m, 1H), 7.27 (m, 1H), 7.43 (m, 1H), 7.61 (m, 1H), 7.89 (m, 1H)

### EXAMPLE 82

### Preparation of 1-n-propyl-2-(2-fluorophenyl)methyl-3,4-dihydro-6-methoxyisoquinolinium chloride (Compound No. 82)

N-Butyryl chloride, instead of acetyl chloride of Example 80, was treated by the same process described in Example 80, to give 1.12 g of oily compound, 1-n-propyl-2-(2-fluorophenyl)methyl-3,4-dihydro-6-methoxy-isoquinolinium chloride.

¹H-NMR (CDCl₃, 300 MHz): δ 1.04 (t, 3H), 1.65 (m, 2H), 3.16 (t, 2H), 3.35 (t, 3H), 3.96 (s, 3H), 4.08 (t, 2H), 5.51 (s, 2H), 6.94 (m, 2H), 7.10 (m, 1H), 7.27 (m, 1H), 7.42 (m, 1H), 7.75 (m, 2H)

### EXAMPLE 83

### Preparation of 1-i-propyl-2-(2-fluorophenyl)methyl-3,4-dihydro-6-methoxyisoquinolinium chloride (Compound No. 83)

Isobutyryl chloride, instead of acetyl chloride of Example 80, was treated by the same process described in Example 80, to give 1.25 g of oily compound, 1-i-propyl-2-(2-fluorophenyl)methyl-3,4-dihydro-6-methoxyisoquinolinium chloride (m.p. 102° C.).

¹H-NMR (CDCl₃, 300 MHz): δ 1.59 (d, J=6.9 Hz, 6H), 3.51 (m, 2H), 3.66 (m, 2H), 3.66 (m, 1H), 3.93 (s, 3H), 4.07 (m, 2H), 5.66 (s, 2H), 6.91 (m, 2H), 7.09 (m, 1H), 7.28 (m, 1H), 7.40 (m, 1H), 7.78 (m, 1H), 7.97 (m, 1H)

### EXAMPLE 84

### Preparation of 1-(2-methyl)propyl-2-(2-fluorophenyl)methyl-3,4-dihydro-6-methoxyisoquinolinium chloride (Compound No. 84)

Isovaleryl chloride, instead of acetyl chloride of Example 80, was treated by the same process described in Example 80, to give 0.91 g of oily compound, 1-(2-methyl)propyl-2-(2-fluorophenyl)methyl-3,4-dihydro-6-methoxyisoquinolinium chloride.

¹H-NMR (CDCl₃, 300 MHz): δ 0.99 (s, 3H), 1.01 (s, 3H), 2.17 (m, 1H), 3.24 (br t, 2H), 3.38 (m, 2H), 3.87 (br t, 2H), 3.94 (s, 3H), 5.65 (s, 2H), 6.87 (d, 1H, J=2.1 Hz), 6.97 (m, 1H), 7.12 (m, 1H), 7.24 (m 1H), 7.38 (m, 1H), 7.81 (m, 2H)

### EXAMPLE 85

### Preparation of 1-n-pentyl-2-(2-fluorophenyl)methyl-3,4-dihydro-6-methoxyisoquinolinium chloride (Compound No. 85)

Caproyl chloride, instead of acetyl chloride of Example 80, was treated by the same process described in Example 80, to give 1.10 g of oily compound, 1-n-pentyl-2-(2-fluorophenyl)methyl-3,4-dihydro-6-methoxyisoquinolinium chloride.

¹H-NMR (CDCl₃, 300 MHz): δ 0.87 (t, 3H), 1.47 (m, 4H), 1.55 (m, 2H), 3.16 (t, 2H), 3.21 (t, 2H), 3.95 (s, 3H), 4.20 (t, 2H), 5.45 (s, 2H), 6.89 (d, J=2.0 Hz, 1H), 6.95 (m, 1H), 7.13 (m, 1H), 7.28 (m, 1H), 7.43 (m, 1H), 7.67 (m, 2H)

### EXAMPLE 86

### Preparation of 1-n-hexyl-2-(2-fluorophenyl)methyl-3,4-dihydro-6-methoxyisoquinolinium chloride (Compound No. 86)

Heptanoyl chloride, instead of acetyl chloride of Example 80, was treated by the same process described in Example 80, to give 1.13 g of oily compound, 1-n-hexyl-2-(2-fluorophenyl)methyl-3,4-dihydro-6-methoxyisoquinolinium chloride.

¹H-NMR (CDCl₃, 300 MHz): δ 0.88 (t, 3H), 1.24 (m, 4H), 1.46 (m, 2H), 1.53 (m, 2H), 3.14 (br t, 2H), 3.30 (m, 2H), 3.94 (s, 3H), 4.11 (br t, 2H), 5.49 (s, 2H), 6.89 (d, J=2.1 Hz, 1H), 6.95 (m, 1H) 7.11 (m, 1H), 7.25 (m, 1H), 7.46 (m, 1H), 7.77 (m, 2H)

### EXAMPLE 87

### Preparation of 1-n-heptyl-2-(2-fluorophenyl)methyl-3,4-dihydro-6-methoxyisoquinolinium chloride (Compound No. 87)

Octanoyl chloride, instead of acetyl chloride of Example 80, was treated by the same process described in Example 80, to give 1.06 g of oily compound, 1-n-heptyl-2-(2-fluorophenyl)methyl-3,4-dihydro-6-methoxyisoquinolinium chloride.

¹H-NMR (CDCl₃, 300 MHz): δ 0.86 (t, J=5.4 Hz, 3H), 1.23 (m, 6H), 1.42 (m, 2H), 1.57 (m, 2H), 3.18 (t, 2H), 3.27 (t, 2H), 3.93 (s, 3H), 4.23 (t, 2H), 5.54 (s, 2H), 6.89 (d, J=2.1 Hz, 1H), 6.95 (m, 1H), 7.10 (m, 1H), 7.27 (m, 1H), 7.39 (m, 1H), 7.81 (m, 2H)

### EXAMPLE 88

### Preparation of 1-n-undecyl-2-(2-fluorophenyl)methyl-3,4-dihydro-6-methoxyisoquinolinium chloride (Compound No. 88)

Lauroyl chloride, instead of acetyl chloride of Example 80, was treated by the same process described in Example 80, to give 1.16 g of oily compound, 1-n2-undecyl-2-(2-fluorophenyl)methyl-3,4-dihydro-6-methoxyisoquinolinium chloride.

¹H-NMR (CDCl₃, 300 MHz): δ 0.87 (t, 3H), 1.22 (m, 14H), 1.41 (m, 2H), 1.56 (m, 2H), 3.14 (br t, 2H), 3.32 (br t, 2H), 3.95 (s, 3H), 4.11 (br t, 2H), 5.51 (s, 2H), 6.89 (d, J=2.0 Hz, 1H), 6.95 (dd, J=2.0, 8.4 Hz, 1H), 7.12 (m, 1H), 7.29 (m, 1H), 7.42 (m, 1H), 7.86 (m, 2H)

### EXAMPLE 89

### Preparation of 1-n-pentadecyl-2-(2-fluorophenyl)methyl-3,4-dihydro-6-methoxyisoquinolinium chloride (Compound No. 89)

Palmitoyl chloride, instead of acetyl chloride of Example 80, was treated by the same process described in Example 80, to give 1.07 g of oily compound, 1-n-pentadecyl-2-(2-fluorophenyl)methyl-3,4-dihydro-6-methoxyisoquinolinium chloride.

¹H-NMR (CDCl₃, 300 MHz): δ 0.86 (t, 3H), 1.24 (m, 22H), 1.42 (m, 2H), 1.59 (m, 2H), 3.18 (br t, 2H), 3.32 (m 2H), 3.95 (s, 3H), 4.12 (br t, 2H), 5.55 (s, 2H), 6.89 (d, J=2.1 Hz, 1H), 6.95 (m, 1H), 7.14 (m, 1H), 7.28 (m, 2H), 7.44 (m, 1H), 7.83 (m, 2H)

### EXAMPLE 90

### Preparation of 1-(2-fluorophenyl)-2-(2-fluorophenyl)methyl-3,4-dihydro-6-methoxyisoquinolinium chloride (Compound No. 90)

2-Fluorobenzoyl chloride, instead of acetyl chloride of Example 80, was treated by the same process described in Example 80, to give 1.03 g of oily compound, 1-(2-fluorophenyl)-2-(2-fluorophenyl)methyl-3,4-dihydro-6-methoxy-isoquinolinium chloride.

¹H-NMR (CDCl₃, 300 MHz): δ 2.99 (m, 1H), 3.34 (m, 1H), 3.92 (s, 3H), 4.09 (m, 1H), 4.86 (m, 1H), 5.35 (d, 1H, J=11.7 Hz), 5.60 (d, 1H, J=11.7 Hz), 6.78 (m, 1H), 6.88 (m, 1H), 7.07 (m, 2H), 7.17 (m, 1H), 7.23 (m, 1H), 7.35 (m, 1H), 7.51 (m, 2H), 7.67 (m, 1H), 8.45 (m, 1H)

### EXAMPLE 91

### Preparation of 1-(4-t-butylphenyl)-2-(2-fluorophenyl)methyl-3,4-dihydro-6-methoxyisoquinolinium chloride (Compound No. 91)

4-t-Butylbenzoyl chloride, instead of acetyl chloride of Example 80, was treated by the same process described in Example 80, to give 1.45 g of oily compound, 1-(4-t-butylphenyl)-2-(2-fluorophenyl)methyl-3,4-dihydro-6-methoxyisoquinolinium chloride.

¹H-NMR (CDCl₃, 300 MHz): δ 1.39 (s, 9H), 3.16 (m, 2H), 3.92 (s, 3H), 4.23 (m, 2H), 5.33 (s, 2H), 6.79 (dd, J=2.4, 9.0 Hz, 1H), 6.87 (d, J=2.4 Hz, 1H), 7.05 (m, 2H), 7.20 (m, 2H), 7.38 (m, 1H), 7.59 (m, 5H)

### EXAMPLE 92

### Preparation of 1-methyl-2-(2-fluorophenyl)methyl-3,4-dihydro-6,7-methylenedioxyisoquinolinium chloride (Compound No. 92)

9.97 G of 3,4-methylenedioxyphenethylamine, instead of 3,4-dimethoxyphenethylamine of Example 1, was treated by the same process described in Example 1, to synthesis 0.82 g of N-(2′-fluorophenyl)methyl-3,4-methylenedioxyphenethylamine. The resulting mixture was dissolved in 25 ml of 1,2-dichloroethane and 0.50 ml of triethylamine was added thereto. Then, 0.26 ml of acetyl chloride was added dropwise slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.46 ml of phosphoryl chloride was added thereto, and the reaction mixture was heated for 8 hours under reflux and cooled to room temperature. The solvent was removed under reduced pressure, and the reaction mixture was separated through silica-gel column chromatography eluting with dichloromethane and methanol (10:1), to give 0.78 g of oily compound, 1-methyl-2-(2-fluorophenyl)methyl-3,4-dihydro-6,7-methylenedioxyisoquinolinium chloride.

¹H-NMR (CDCl₃, 300 MHz): δ 3.08 (s, 3H), 3.08 (m, 2H), 4.11 (m, 2H), 5.72 (s, 2H), 6.10 (s, 2H), 6.96 (s, 1H), 7.11 (m, 1H), 7.28 (s, 1H), 7.44 (m, 1H), 7.86 (m, 2H)

### EXAMPLE 93

### Preparation of 1-ethyl-2-(2-fluorophenyl)methyl-3,4-dihydro-6,7-methylenedioxyisoquinolinium chloride (Compound No. 93)

Propionyl chloride, instead of acetyl chloride of Example 92, was treated by the same process described in Example 92, to give 0.96 g of oily compound, 1-ethyl-2-(2-fluorophenyl)methyl-3,4-dihydro-6,7-methoxyisoquinolinium chloride.

¹H-NMR (CDCl₃, 300 MHz): δ 1.36 (t, 3H), 3.13 (t, 2H), 3.37 (m, 2H), 4.15 (m, 2H), 5.69 (s, 2H), 6.11 (s, 2H), 6.85 (s, 1H), 7.01 (m, 1H), 7.27 (m, 1H), 7.43 (m, 1H), 7.59 (m, 1H), 7.87 (m, 1H)

### EXAMPLE 94

### Preparation of 1-n-propyl-2-(2-fluorophenyl)methyl-3,4-dihydro-6,7-methylenedioxyisoquinolinium chloride (Compound No. 94)

n-Butyryl chloride, instead of acetyl chloride of Example 92, was treated by the same process described in Example 92, to give 1.12 g of oily compound, 1-n-propyl-2-(2-fluorophenyl)methyl-3,4-dihydro-6,7-methoxyisoquinolinium chloride.

¹H-NMR (CDCl₃, 300 (MHz): δ 1.01 (t, 3H), 1.32 (s, 2H), 1.64 (m, 2H), 3.16 (m, 2H), 4.08 (m, 2H), 5.55 (s, 2H), 6.12 (s, 2H), 6.94 (m, 1H), 7.10 (m, 1H), 7.29 (m, 1H), 7.44 (m, 1H), 7.75 (m, 2H)

### EXAMPLE 95

### Preparation of 1-i-propyl-2-(2-fluorophenyl)methyl-3,4-dihydro-6,7-methylenedioxyisoquinolinium chloride (Compound No. 95)

i-Butyryl chloride, instead of acetyl chloride of Example 92, was treated by the same process described in Example 92, to give 1.25 g of solid compound, 1-i-propyl-2-(2-fluorophenyl)methyl-3,4-dihydro-6,7-methylenedioxyisoquinolinium chloride (m.p. 114° C.).

¹H-NMR (CDCl₃, 300 MHz): δ 1.57 (d, J=6.9 Hz, 6H), 3.50 (m, 2H), 3.64 (m, 2H), 3.67 (m, 1H), 5.63 (s, 2H), 6.11 (s, 2H), 6.91 (m, 1H), 7.01 (s, 1H), 7.28 (s, 1H), 7.40 (m, 1H), 7.78 (m, 1H), 7.97 (m, 1H)

### EXAMPLE 96

### Preparation of 1-n-pentyl-2-(2-fluorophenyl)methyl-3,4-dihydro-6,7-methylenedioxyisoquinolinium chloride (Compound No. 96)

Hexanoyl chloride, instead of acetyl chloride of Example 92, was treated by the same process described in Example 92, to give 1.10 g of oily compound, 1-n-pentyl-2-(2-fluorophenyl)methyl-3,4-dihydro-6,7-methylenedioxyisoquinolinium chloride.

¹H-NMR (CDCl₃, 300 MHz): δ 0.88 (t, 3H), 1.49 (m, 4H), 1.54 (m, 2H), 3.14 (m, 2H), 3.21 (m, 2H), 4.23 (m, 2H), 5.48 (s, 2H), 6.09 (s, 2H), 6.92 (s, 1H), 7.14 (m, 1H), 7.17 (m, 1H), 7.28 (m, 1H), 7.43 (m, 1H), 7.78 (m, 1H)

### EXAMPLE 97

### Preparation of 1-n-hexyl-2-(2-fluorophenyl)methyl-3,4-dihydro-6,7-methylenedioxyisoquinolinium chloride (Compound No. 97)

Heptanoyl chloride, instead of acetyl chloride of Example 92, was treated by the same process described in Example 92, to give 1.10 g of oily compound, 1-n-hexyl-2-(2-fluorophenyl)methyl-3,4-dihydro-6,7-methoxyisoquinolinium chloride.

¹H-NMR (CDCl₃, 300 MHz): δ 0.87 (t, 3H), 1.24 (m, 4H), 1.44 (m, 2H), 1.51 (m, 2H), 3.17 (m, 2H), 3.28 (m, 2H), (4.12m, 2H), 5.51 (s, 2H), 6.10 (s, 2H), 6.97 (s, 1H), 7.11 (m, 1H), 7.25 (m, 1H), 7.46 (m, 1H), 7.77 (m, 1H), 7.84 (m, 1H)

### EXAMPLE 98

### Preparation of 1-n-heptyl-2-(2-fluorophenyl)methyl-3,4-dihydro-6,7-methylenedioxyisoquinolinium chloride (Compound No. 98)

Octanoyl chloride, instead of acetyl chloride of Example 92, was treated by the same process described in Example 92, to give 1.06 g of solid compound, 1-n-heptyl-2-(2-fluorophenyl)methyl-3,4-dihydro-6,7-methylenedioxyisoquinolinium chloride (m.p. 94° C.).

¹H-NMR (CDCl₃, 300 MHz): δ 0.87 (t, 3H), 1.23 (m, 6H), 1.42 (m, 2H), 1.54 (m, 2H), 3.15 (t, 2H), 3.24 (t, 2H), 4.04 (m, 2H), 5.56 (s, 2H), 6.11 (s, 2H), 6.94 (m, 1H), 7.14 (m, 1H), 7.28 (m, 1H), 7.39 (m, 1H), 7.48 (m, 1H), 7.81 (m, 1H)

### EXAMPLE 99

### Preparation of 1-n2-undecyl-2-(2-fluorophenyl)methyl-3,4-dihydro-6,7-methylenedioxyisoquinolinium chloride (Compound No. 99)

Lauroyl chloride, instead of acetyl chloride of Example 92, was treated by the same process described in Example 92, to give 1.16 g of oily compound, 1-n-undecyl-2-(2-fluorophenyl)methyl-3,4-dihydro-6,7-methylenedioxyisoquinolinium chloride.

¹H-NMR (CDCl₃, 300 MHz): δ 0.88 (t, 3H), 1.23 (m, 14H), 1.41 (m, 2H), 1.56 (m, 2H), 3.14 (m, 2H), 3.32 (m, 2H), 4.10 (m, 2H), 5.57 (s, 2H), 6.11 (s, 2H), 6.92 (s, 1H), 7.12 (m, 1H), 7.29 (m, 1H), 7.42 (m, 1H), 7.54 (m, 1H), 7.86 (m, 1H)

### EXAMPLE 100

### Preparation of 1-n-pentadecyl-2-(2-fluorophenyl)methyl-3,4-dihydro-6,7-methylenedioxyisoquinolinium chloride (Compound No. 100)

Palmitoyl chloride, instead of acetyl chloride of Example 92, was treated by the same process described in Example 92, to give 1.07 g of oily compound, 1-n-pentadecyl-2-(2-fluorophenyl)methyl-3,4-dihydro-6,7-methylenedioxyisoquinolinium chloride.

¹H-NMR (CDCl₃, 300 MHz): δ 0.88 (t, 3H), 1.22 (m, 22H), 1.43 (m, 2H), 1.59 (m, 2H), 3.18 (m, 2H), 3.32 (m 2H), 4.12 (m, 2H), 5.55 (s, 2H), 6.10 (s, 2H), 6.89 (s, 1H), 6.95 (m, 1H), 7.14 (m, 1H), 7.28 (m, 1H), 7.44 (m, 1H), 7.83 (m, 2H)

### EXAMPLE 101

### Preparation of 1-(2-fluorophenyl)-2-(2-fluorophenyl)methyl-3,4-dihydro-6,7-methylenedioxyisoquinolinium chloride (Compound No. 101)

2-Fluorobenzoyl chloride, instead of acetyl chloride of Example 92, was treated by the same process described in Example 92, to give 1.03 g of oily compound, 1-(2-fluorophenyl)-2-(2-fluorophenyl)methyl-3,4-dihydro-6,7-methylenedioxyisoquinolinium chloride.

¹H-NMR (CDCl₃, 300 MHz): δ 3.18 (m, 1H), 3.39 (m, 1H), 4.11 (m, 1H), 4.83 (m, 1H), 5.35 (d, J=12 Hz, 1H), 5.54 (d, J=12 Hz, 1H), 6.11 (s, 2H), 6.44 (s, 1H), 6.99 (s, 1H), 7.07 (m, 1H), 7.17 (m, 1H), 7.32 (m, 1H), 7.40 (m, 1H), 7.49 (m, 2H), 7.70 (m, 1H), 7.85 (m, 1H)

### EXAMPLE 102

### Preparation of 1-(3,4-difluorophenyl)-2-(2-fluorophenyl)methyl-3,4-dihydro-6,7-methylenedioxyisoquinolinium chloride (Compound No. 102)

3,4-Difluorobenzoyl chloride, instead of acetyl chloride of Example 92, was treated by the same process described in Example 92, to give 1.11 g of oily compound, 1-(3,4-difluorophenyl)-2-(2-fluorophenyl)methyl-3,4-dihydro-6,7-methylenedioxyisoquinolinium chloride.

¹H-NMR (CDCl₃, 300 MHz): δ 3.22 (t, 2H), 4.38 (t, 2H), 5.42 (s, 2H), 6.09 (s, 2H), 6.38 (s, 1H), 7.05 (s, 1H), 7.11 (m, 1H), 7.22 (m, 1H), 7.49 (m, 3H), 7.85 (m, 1H), 8.02 (m, 1H)

### EXAMPLE 103

### Preparation of 1-(3-chlorophenyl)-2-(2-fluorophenyl)methyl-3,4-dihydro-6,7-methylenedioxyisoquinolinium chloride (Compound No. 103)

3-Chlorobenzoyl chloride, instead of acetyl chloride of Example 92, was treated by the same process described in Example 92, to give 1.21 g of oily compound, 1-(3-chlorophenyl)-2-(2-fluorophenyl)methyl-3,4-dihydro-6,7-methylenedioxyisoquinolinium chloride.

¹H-NMR (CDCl₃, 300 MHz): δ 3.24 (m, 2H), 4.30 (m, 1H), 4.53 (m, 1H), 5.45 (dd, 2H), 6.09 (s, 2H), 6.41 (s, 1H), 6.98 (s, 1H), 7.08 (t, 1H), 7.20 (t, 1H), 7.41 (m, 1H), 7.52 (t, 1H), 7.63 (m, 2H), 7.76 (s, 1H), 8.05 (m, 1H)

### EXAMPLE 104

### Preparation of 1-(4-chlorophenyl)-2-(2-fluorophenyl)methyl-3,4-dihydro-6,7-methylenedioxyisoquinolinium chloride (Compound No. 104)

4-Chlorobenzoyl chloride, instead of acetyl chloride of Example 92, was treated by the same process described in Example 92, to give 1.23 g of 1-(4-chlorophenyl)-2-(2-fluorophenyl)methyl-3,4-dihydro-6,7-methylenedioxyisoquinolinium chloride.

¹H-NMR (CDCl₃, 300 MHz): δ 3.17 (t, 2H), 4.37 (t, 2H), 5.45 (s, 2H), 6.11 (s, 2H), 6.45 (s, 1H), 6.89 (s, 1H), 7.04 (t, 1H), 7.21 (t, 1H), 7.38 (m, 1H), 7.60 (m, 3H), 7.89 (m, 2H)

### EXAMPLE 105

### Preparation of 1-(4-n-butylphenyl)-2-(2-fluorophenyl)methyl-3,4-dihydro-6,7-methylenedioxyisoquinolinium chloride (Compound No. 105)

4-n-Butylbenzoyl chloride, instead of acetyl chloride of Example 92, was treated by the same process described in Example 92, to give 1.31 g of solid compound, 1-(4-n-butylphenyl)-2-(2-fluorophenyl)methyl-3,4-dihydro-6,7-methylenedioxyisoquinolinium chloride (m.p. 101° C.).

¹H-NMR (CDCl₃, 300 MHz): δ 0.94 (t, 3H), 1.37 (m, 2H), 1.65 (m, 2H), 2.74 (t, 2H), 3.16 (m, 2H), 4.34 (m, 2H), 5.53 (s, 2H), 6.10 (s, 2H), 6.42 (s, 1H), 6.85 (s, 1H), 7.01 (t, 1H), 7.16 (t, 1H), 7.37 (m, 1H), 7.45 (d, J=6.0 Hz, 2H), 7.62 (t, 1H), 7.73 (d, J=6.0 Hz, 2H)

### EXAMPLE 106

### Preparation of 1-(4-t-butylphenyl)-2-(2-fluorophenyl)methyl-3,4-dihydro-6,7-methylenedioxyisoquinolinium chloride (Compound No. 106)

4-t-Butylbenzoyl chloride, instead of acetyl chloride of Example 92, was treated by the same process described in Example 92, to give 1.45 g of solid compound, 1-(4-t-butylphenyl)-2-(2-fluorophenyl)methyl-3,4-dihydro-6,7-methylenedioxyisoquinolinium chloride (m.p. 148° C.).

¹H-NMR (CDCl₃, 300 MHz): δ 1.39 (s, 9H), 3.18 (t, 2H), 4.01 (t, 2H), 5.33 (s, 2H), 6.11 (s, 2H), 6.46 (s, 1H), 6.88 (s, 1H), 7.06 (m, 1H), 7.22 (m, 1H), 7.38 (m, 1H), 7.51 (m, 1H), 7.60 (d, J=9.0 Hz, 2H), 7.66 (d, J=9.0 Hz, 2H)

### EXAMPLE 107

### Preparation of 1-methyl-2-(4-trifluoromethylphenyl)methyl-3,4-dihydro-6,7-methylenedioxyisoquinolinium chloride (Compound No. 107)

3,4-Methylenedioxyphenethylamine, instead of 3,4-dimethoxyphenethylamine of Example 26, was treated by the same process described in Example 26, to give N-(4-trifluoromethylphenyl)methyl-3,4-methylenedioxyphenethylamine. The resulting mixture was dissolved in 25 ml of 1,2-dichloroethane and 0.50 ml of triethylamine was added thereto. Then, 0.26 ml of acetyl chloride was added dropwise slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.46 ml of phosphoryl chloride was added thereto, and the reaction mixture was heated for 8 hours under reflux and cooled to room temperature. The solvent was removed under reduced pressure, and the reaction mixture was separated through silica-gel column chromatography eluting with dichloromethane and methanol (10:1), to give 1.02 g of oily compound, 1-methyl-2-(4-trifluoromethylphenyl)methyl-3,4-dihydro-6,7-methylenedioxyisoquinolinium chloride.

¹H-NMR (CDCl₃, 300 MHz): δ 3.01 (s, 3H), 3.13 (m, 2H), 4.04 (m, 2H), 5.50 (s, 2H), 6.10 (s, 2H), 6.82 (s, 1H), 9.34 (s, 1H), 7.50 (d, 2H), 7.66 (d, 2H)

### EXAMPLE 108

### Preparation of 1-n-heptyl-2-(4-trifluoromethylphenyl)methyl-3,4-dihydro-6,7-methylenedioxyisoquinolinium chloride (Compound No. 108)

Octanoyl chloride, instead of acetyl chloride of Example 107, was treated by the same process described in Example 107, to give 1.13 g of oily compound, 1-n-heptyl-2-(4-trifluoromethylphenyl)methyl-3,4-dihydro-6,7-methylenedioxyisoquinolinium chloride.

¹H-NMR (CDCl₃, 300 MHz): δ 0.82 (t, 3H), 1.21 (m, 6H), 1.25 (m, 2H), 1.34 (m, 2H), 3.16 (m, 2H), 3.25 (m, 2H), 4.10 (m, 2H), 5.57 (s, 2H), 6.11 (s, 2H), 6.99 (s, 1H), 7.28 (s, 1H), 7.54 (d, J=9.0 Hz, 2H), 7.63 (d, J=9.0 Hz, 2H)

### EXAMPLE 109

### Preparation of 1-n-pentadecyl-2-(4-trifluoromethylphenyl)methyl-3,4-dihydro-6,7-methylenedioxyisoquinolinium chloride (Compound No. 109)

Palmitoyl chloride, instead of caproyl chloride of Example 107, was treated by the same process described in Example 107, to give 1.07 g of oily compound, 1-n-pentadecyl-2-(4-trifluoromethylphenyl)methyl-3,4-dihydro-6,7-methylenedioxyisoquinolinium chloride.

¹H-NMR (CDCl₃, 300 MHz): δ 0.86 (t, 3H), 1.23 (m, 22H), 1.44 (m, 2H), 1.65 (m, 2H), 3.16 (m, 2H), 3.22 (m 2H), 4.10 (br t, 2H), 5.57 (s, 2H), 6.09 (s, 2H), 6.84 (s, 1H), 7.27 (s, 1H), 7.53 (d, 2H, J=6.0 Hz), 7.71 (d, 2H, J=6.0 Hz)

799

### EXAMPLE 110

### Preparation of 1-(4-t-butylphenyl)-2-(4-trifluoromethylphenyl)methyl-3,4-dihydro-6,7-methylenedioxyisoquinolinium chloride (Compound No. 110)

4-t-Butylbenzoyl chloride, instead of caproyl chloride of Example 107, was treated by the same process described in Example 107, to give 1.32 g of solid compound, 1-(4-t-butylphenyl)-2-(4-trifluoromethylphenyl)methyl-3,4-dihydro-6,7-methylenedioxyisoquinolinium chloride (m.p. 129˜132° C.).

¹H-NMR (CDCl₃, 300 MHz): δ 1.38 (s, 9H), 3.20 (m, 2H), 4.24 (m, 2H), 5.40 (s, 2H), 6.10 (s, 2H), 6.51 (s, 1H), 6.88 (s, 1H), 7.43 (m, 2H), 7.65 (m, 6H)

### EXAMPLE 111

### Preparation of 1-methyl-2-(3,4-difluorophenyl)methyl-3,4-dihydro-6,7-methylenedioxyisoquinolinium chloride (Compound No. 111)

3,4-Methylenedioxyphenethylamine, instead of 3,4-dimethoxyphenethylamine of Example 39, was treated by the same process described in Example 39, to give N-(3,4-difluorophenyl)methyl-3,4-methylenedioxyphenethylamine. The resulting mixture was dissolved in 25 ml of 1,2-dichloroethane and 0.50 ml of triethylamine was added thereto. Then, 0.26 ml of acetyl chloride was added dropwise slowly to the mixture at 0° C., and the reaction mixture was stirred at room temperature for about 1 hour. The reaction mixture was washed with 25 ml of distilled water and separated into organic phase and aqueous phase. The aqueous phase was extracted twice with 25 ml of dichloromethane, and the organic phase thus separated was dried over MgSO₄, filtered and concentrated under reduced pressure to synthesize amide intermediate.

The crude intermediate thus obtained was dissolved in 25 ml of acetonitrile and 0.46 ml of phosphoryl chloride was added thereto, and the reaction mixture was heated for 8 hours under reflux and cooled to room temperature. The solvent was removed under reduced pressure, and the reaction mixture was separated through silica-gel column chromatography eluting with dichloromethane and methanol (10:1), to give 1.17 g of solid compound, 1-methyl-2-(3,4-difluorophenyl)methyl-3,4-dihydro-6,7-methylenedioxyisoquinolinium chloride (m.p. 88° C.).

¹H-NMR (CDCl₃, 300 MHz): δ 3.01 (s, 3H), 3.12 (t, 2H), 4.04 (t, 2H), 5.42 (s, 2H), 6.12 (s, 2H), 6.81 (s, 1H), 7.14-7.24 (m, 4H), 7.32 (s, 1H)

### EXAMPLE 112

### Preparation of 1-n-heptyl-2-(3,4-difluorophenyl)methyl-3,4-dihydro-6,7-methylenedioxyisoquinolinium chloride (Compound No. 112)

Octanoyl chloride, instead of acetyl chloride of Example 111, was treated by the same process described in Example 107, to give 1.18 g of oily compound, 1-n-heptyl-2-(3,4-difluorophenyl)methyl-3,4-dihydro-6,7-methylene-dioxyisoquinolinium chloride (m.p. 129˜132° C.).

¹H-NMR (CDCl₃, 300 MHz): 0.88 (t, 3H), 1.24 (m, 6H), 1.44 (m, 2H), 1.67 (m, 2H), 3.18 (m, 2H), 3.29 (m, 2H), 4.13 (m, 2H), 5.63 (s, 2H), 6.10 (s, 2H), 6.88 (s, 1H) 7.18-7.31 (m, 4H)

### EXAMPLE 113

### Preparation of 1-n-pentadecyl-2-(3,4-difluorophenyl)methyl-3,4-dihydro-6,7-methylenedioxyisoquinolinium chloride (Compound No. 113)

Palmitoyl chloride, instead of acetyl chloride of Example 111, was treated by the same process described in Example 111, to give 1.07 g of oily compound, 1-n-pentadecyl-2-(3,4-difluorophenyl)methyl-3,4-dihydro-6,7-methylenedioxyisoquinolinium chloride.

¹H-NMR (CDCl₃, 300 MHz): δ 0.86 (t, 3H), 1.24 (m, 22H), 1.47 (m, 2H), 1.66 (m, 2H), 3.20 (t, 2H), 3.34 (t, 2H), 4.18 (t, 2H), 5.73 (s, 2H), 6.11 (s, 2H), 6.92 (s, 1H), 7.24 (m, 1H), 7.29 (s, 1H), 7.38 (m, 2H)

### EXAMPLE 114

### Preparation of 1-(4′-t-butylphenyl)-2-(3,4-difluorophenyl)methyl-3,4-dihydro-6,7-methylenedioxyisoquinolinium chloride (Compound No. 114)

4-t-Butylbenzoyl chloride, instead of acetyl chloride of Example 111, was treated by the same process described in Example 11, to give 1.41 g of solid compound, 1-(4-t-butylphenyl)-2-(3,4-difluorophenyl)methyl-3,4-dihydro-6,7-methylenedioxyisoquinolinium chloride (m.p. 182° C.).

¹H-NMR (CDCl₃, 300 MHz): δ 1.39 (s, 9H), 3.20 (m, 2H), 4.13 (m, 2H), 5.13 (s, 2H), 6.11 (s, 2H), 6.44 (s, 1H), 6.90 (m, 1H), 6.99 (m, 1H), 7.15 (m, 2H), 7.67 (m, 4H)

### EXAMPLE 115

### Preparation of 7,8-dihydro-1-methyl-6-(4-t-butylphenyl)methyl-5-undecyloxazolo[4,5-g]isoquinolinium-2 (1H)-one bromide (Compound No. 115)

5 ML of acetonitrile solution was added to 356 mg of 7,8-dihydro-1-methyl-5-undecyloxazolo[4,5-g]isoquinoline-2 (1H)-one and 227 mg of 1-t-butyl-4-(bromomethyl)benzene. Then, the resulting mixture was heated and stirred for 5 hours. The solvent was concentrated under reduced pressure, and the reaction mixture was separated through silica-gel column chromatography eluting with dichloromethane and methanol (10:1), to give 0.45 g of solid compound, 7,8-dihydro-1-methyl-6-(4-t-butylphenyl)methyl-5-undecyloxazolo[4,5-g]isoquinolinium-2 (1H)-one bromide (m.p. 116° C.).

¹H-NMR (CDCl₃, 300 MHz): δ 0.84 (t, 3H), 1.21 (b, 14H), 1.28 (s, 9H), 1.44 (b, 2H), 1.67 (b, 2H), 3.27 (t, 2H), 3.29 (t, 2H), 3.48 (s, 3H), 4.12 (t, 2H), 5.48 (s, 2H), 7.28 (d, 2H), 7.38 (d, 2H), 7.43 (s, 1H), 7.66 (s, 1H)

### EXAMPLE 1116

### Preparation of ethyl 3,4-dihydro-7-methoxy-6-(4-t-butylphenyl)-1-undecylisoquinolinium-6-ylmethylcabamate bromide (Compound No. 116)

5 ML of acetonitrile solution was added to 417 mg of ethyl 3,4-dihydro-7-methoxy-1-undecylisoquinolin-6-ylmethylcabamate and 227 mg of 1-t-butyl-4-(bromomethyl)benzene. Then, the resulting mixture was heated and stirred for 5 hours. The solvent was concentrated under reduced pressure, and the reaction mixture was separated through silica-gel column chromatography eluting with dichloromethane and methanol (10:1), to give 0.50 g of solid compound, ethyl 3,4-dihydro-7-methoxy-6-(4-t-butylphenyl)-1-undecylisoquinolinium-6-ylmethylcabamate bromide

(m.p. 145° C.).

¹H-NMR (CDCl₃, 300 MHz): δ 0.84 (t, 3H), 1.21 (b, 14H), 1.26 (t, 3H), 1.28 (s, 9H), 1.44 (b, 2H), 1.67 (b, 2H), 3.27 (t, 2H), 3.29 (t, 2H), 3.32 (s, 3H), 4.12 (t, 2H), 4.20 (q, 2H), 5.48 (s, 2H), 7.28 (d, 2H), 7.38 (d, 2H), 7.43 (s, 1H), 7.66 (s, 1H)

### EXAMPLE 117

### Preparation of Ethyl 3,4-dihydro-1-undecylisoquinolinium-2-(4-trifluoromethylphenyl)-6-ylmethylcabamate bromide (Compound No. 117)

5 Ml of acetonitrile solution was added to 386 mg of ethyl 3,4-dihydro-1-undecylisoquinolin-6-ylmethylcabamate and 195 mg of 1-trifluoromethyl-4-(bromomethyl)benzene. Then, the resulting mixture was heated and stirred for 5 hours. The solvent was concentrated under reduced pressure, and the reaction mixture was separated through silica-gel column chromatography eluting with dichloromethane and methanol (10:1), to give 0.40 g of solid compound, ethyl 3,4-dihydro-1-undecylisoquinolinium-2-(4-trifluoromethylphenyl)-6-ylmethylcabamate bromide (m.p. 127° C.).

¹H-NMR (CDCl₃, 300 MHz): δ 0.79 (t, 3H), 1.18 (b, 17H), 1.36 (b, 2H), 1.59 (b, 2H), 3.02 (t, 2H), 3.10 (t, 2H), 3.99 (t, 2H), 4.13 (q, 2H), 5.52 (s, 2H), 7.53 (d, 2H), 7.62 (d, 2H), 7.78 (s, 1H), 7.91 (d, 1H), 9.89 (b, 1H)

### EXAMPLE 118

### Preparation of ethyl 3,4-dihydro-2-(4-t-butylphenyl)-1-undecylisoquinolinium-6-ylmethylcabamate bromide (Compound No. 118)

5 ML of acetonitrile solution was added to 386 mg of ethyl 3,4-dihydro-1-undecylisoquinolinium-6-ylmethylcabamate and 227 mg of 1-t-butyl-4-(bromomethyl)benzene. Then, the resulting mixture was heated and stirred for 5 hours. The solvent was concentrated under reduced pressure, and the reaction mixture was separated through silica-gel column chromatography eluting with dichloromethane and methanol (10:1), to give 0.41 g of solid compound, ethyl 3,4-dihydro-1-undecylisoquinolinium-2-(4-trifluoromethylphenyl)-6-ylmethylcabamate bromide (m.p. 134° C.).

¹H-NMR (CDCl₃, 300 MHz): δ 0.79 (t, 3H), 1.18 (b, 17H), 1.32 (s, 9H), 1.36 (b, 2H), 1.59 (b, 2H), 3.02 (t, 2H), 3.10 (t, 2H), 3.99 (t, 2H), 4.13 (q, 2H), 5.52 (s, 2H), 7.24 (d, 2H), 7.44 (d, 2H), 7.78 (s, 1H), 7.91 (d, 1H), 9.62 (b, 1H)

### EXAMPLE 119

### Preparation of 2-(4-t-butylphenyl)-3,4-dihydro-7-methoxy-N-methyl-1-undecylisoquinolinium-6-amine bromide (Compound No. 119)

5 Ml of acetonitrile solution was added to 386 mg of ethyl 3,4-dihydro-7-methoxy-N-methyl-1-undecylisoquinolinium-6-amine and 227 mg of 1-t-butyl-4-(bromomethyl)benzene. Then, the resulting mixture was heated and stirred for 5 hours. The solvent was concentrated under reduced pressure, and the reaction mixture was separated through silica-gel column chromatography eluting with dichloromethane and methanol (10:1), to give 0.50 g of solid compound, 2-(4-t-butylphenyl)-3,4-dihydro-7-methoxy-N-methyl-1-undecylisoquinolinium-6-amine bromide (m.p. 134° C.).

¹H-NMR (CDCl₃, 300 MHz): δ 0.84 (t, 3H), 1.21 (b, 14H), 1.28 (s, 9H), 1.44 (b, 2H), 1.67 (b, 2H), 3.27 (t, 2H), 3.29 (t, 2H), 3.41 (s, 3H), 3.89 (s, 3H), 4.12 (t, 2H), 5.48 (s, 2H), 6.36 (s, 1H), 6.97 (s, 1H), 7.24 (d, 2H), 7.38 (d, 2H)

### EXAMPLE 120

### Preparation of 2-(4-t-butylalcohol carbonylaminophenyl)methyl-3,4-dihydro-6,7-dimethoxy-1-undecylisoquinolinium chloride (Compound No. 120)

5 ML of acetonitrile solution was added to 345 mg of 3,4-dihydro-6,7-dimethoxy-1-undecylisoquinoline-6-amine and 242 mg of 1-t-butyl-3-chloromethyl)phenylcarbamate. Then, the resulting mixture was heated and stirred for 5 hours. The solvent was concentrated under reduced pressure, and the reaction mixture was separated through silica-gel column chromatography eluting with dichloromethane and methanol (10:1), to give 0.40 g of solid compound, 2-(4-t-butylalcohol carbonylaminophenyl)methyl-3,4-dihydro-6,7-dimethoxy-1-undecylisoquinolinium chloride (m.p. 142° C.).

¹H-NMR (CDCl₃, 300 MHz): δ 0.84 (t, 3H), 1.20 (b, 14H), 1.45 (s, 9H), 1.62 (b, 4H), 3.10 (t, 2H), 3.26 (t, 2H), 3.92 (s, 3H), 3.97 (s, 3H), 4.01 (t, 2H), 5.34 (s, 2H), 6.82 (s, 1H), 7.27 (s, 1H), 7.60 (d, 1H), 7.67 (s, 1H), 7.96 (s, 1H)

### EXAMPLE 121

### Preparation of 3,4-dihydro-6,7-dimethoxy-2-(4-ammoniumphenyl)methyl-1-undecylisoquinolinium dichloride (Compound No. 121)

0.20 G of 3,4-dihydro-6,7-dimethoxy-2-(4-t-butylalcohol carbonylaminophenyl)methyl-1-undecylisoquinolinium chloride was dissolved in 3 ml of dichloromethane, and 0.5 ml of trifluoro acetic acid was added thereto at room temperature. Then, the resulting mixture was stirred for 5 hours and the solvent was concentrated to prepare 0.25 g of 3,4-dihydro-6,7-dimethoxy-2-(4-ammoniumphenyl)methyl-1-undecylisoquinolinium dichloride (m.p. 150° C.).

¹H-NMR (CDCl₃, 300 MHz): δ 0.84 (t, 3H), 1.20 (b, 14H), 1.62 (b, 4H), 3.10 (t, 2H), 3.26 (t, 2H), 3.92 (s, 3H), 3.97 (s, 3H), 4.01 (t, 2H), 5.34 (s, 2H), 6.82 (s, 1H), 7.27 (s, 1H), 7.60 (d, 1H), 7.67 (s, 1H), 7.96 (s, 1H)

### EXAMPLE 122

### Preparation of 6,7-dimethoxy-2-(3,4-bis(ethoxy Carbonylamino)phenyl)methyl-3,4-dihydro-1-undecylisoquinolinium chloride (Compound No. 122)

15 ML of acetonitrile solution was added to 345 mg of 6,7-dimethoxy-3,4-dihydro-1-undecylisoquinoline and 242 mg of 3,4-bis(ethoxycarbonylamino)benzyl chloride. Then, the resulting mixture was heated and stirred for 5 hours. The solvent was concentrated under reduced pressure, and the reaction mixture was separated through silica-gel column chromatography eluting with dichloromethane and methanol (10:1), to give 0.40 g of solid compound, 6,7-dimethoxy-2-(3,4-diethylacohol carbonylaminophenyl)methyl-3,4-dihydro-1-undecylisoquinolinium chloride (m.p. 147° C.).

¹H-NMR (CDCl₃, 300 MHz): δ 0.84 (t, 3H), 1.20 (b, 20H), 1.62 (b, 4H), 3.10 (t, 2H), 3.26 (t, 2H), 3.92 (s, 3H), 3.97 (s, 3H), 4.01 (t, 2H), 4.12 (q, 4H), 5.34 (s, 2H), 6.78 (d, 2H), 6.82 (s, 1H), 7.27 (s, 1H), 7.42 (s, 1H), 7.50 (d, 1H)

### EXAMPLE 123

### Preparation of 1-propyl-2-(4-t-butylphenyl)methyl-3,4-dihydro-6,7-dimethoxyisoquinolinium chloride (Compound No. 123)

233 Mg of 1-propyl-6,7-dimethoxy-3,4-dihydroisoquinoline was reacted with 218 mg of 4-t-butylbenzylchloride to give 361 mg of 1-propyl-2-(4-t-butylphenyl)methyl-3,4-dihydro-6,7-dimethoxy-isoquinolinium chloride (yield: 87%) (m.p. 100° C.).

¹H-NMR (CDCl₃, 300 MHz): δ 1.11 (t, 3H, J=7.5 Hz), 1.84 (dt, 2H, J=7.8 Hz), 3.20 (t, 2H, J=7.2 Hz), 3.32 (t, 2H, J=7.2 Hz), 3.97 (s, 3H), 4.01 (s, 3H), 4.15 (t, 2H, J=7.8 Hz), 5.51 (s, 2H), 6.88 (s, 1H), 7.29 (d, 2H, J=8.4 Hz), 7.31 (s, 1H), 7.43 (d, 2H, J=8.4 Hz)

### EXAMPLE 124

### Preparation of 1-(2-(4-t-butylphenyl))ethyl-2-(2-fluorophenyl)methyl-3,4-dihydro-6,7-dimethoxyisoquinolinium chloride (Compound No. 124)

3-(4-t-Butylphenyl)propionyl chloride, instead of butyryl chloride of Example 1, was treated by the same process described in Example 1, to give 1.41 g of solid compound, 1-(2-(4-t-butylphenyl))ethyl-3,4-dihydro-6,7-dimethoxyisoquinoline chloride. Then, the resulting mixture was reacted with 2-fluorobenzylchloride to give 421 mg of 1-(2-(4-t-butylphenyl))ethyl-2-(2-fluorophenyl)methyl-3,4-dihydro-6,7-dimethoxyisoquinolinium chloride

(m.p. 103° C.).

¹H-NMR (CDCl₃, 300 MHz): δ 1.27 (s, 9H), 3.03 (t, 2H, J=7.2 Hz), 3.16 (t, 2H, J=7.2 Hz), 3.68 (t, 2H, J=7.5 Hz), 3.82 (s, 3H), 3.97 (s, 3H), 4.11 (t, 2H, J=7.5 Hz), 5.65 (s, 2H), 6.76 (s, 1H), 7.10 (s, 1H), 7.00˜7.30 (m, 6H), 7.30˜7.50 (m, 1H), 7.80˜7.95 (m, 1H)

### EXAMPLE 125

### Preparation of 1-(2-(4-t-butylphenyl))ethyl-2-(3,4-dimethoxyphenyl)methyl-3,4-dihydro-6,7-dimethoxyisoquinolinium chloride (Compound No. 125)

351 Mg of 1-(2-(4-t-butylphenyl))ethyl-2-(2-fluorophenyl)methyl-3,4-dihydro-6,7-dimethoxyisoquinolinine of Example 124 was reacted with 223 mg of 3,4-dimethoxybenzylchloride to give 462 mg of 1-(2-(4-t-butylphenyl))ethyl-2-(3,4-dimethoxyphenyl)methyl-3,4-dihydro-6,7-dimethoxyisoquinoliniumchloride (m.p. 100° C.).

¹H-NMR (CDCl₃, 300 MHz): δ 1.28 (s, 9H), 3.00˜3.20 (m, 4H), 3.65 (t, 2H, J=7.4 Hz), 3.85 (s, 3H), 3.87 (s, 3H), 3.91 (s, 3H), 3.99 (s, 3H), 4.15 (t, 2H, J=7.4 Hz), 5.40 (s, 2H), 6.7˜6.9 (m, 2H), 7.00˜7.20 (m, 1H), 7.08 (d, 2H, J=8.1 Hz), 7.17 (s, 1H), 6.87 (s, 1H), 7.29 (d, 2H, J=8.1 Hz)

### EXAMPLE 126

### Preparation of 1-hexyl-2-(4-t-butylphenyl)methyl-3,4-dihydro-6,7-dimethoxy isoquinolinium chloride (Compound No. 126)

Heptanoyl chloride, instead of butyryl chloride of Example 1, was treated by the same process described in Example 1, to give 11.0 mmol of 1-hexyl-2-(4-t-butylphenyl)methyl-3,4-dihydro-6,7-dimethoxy isoquinoline. Then, the resulting product was reacted with 1.2 mmol of 4-t-butylbenzylchloride to give 389 mg of 1-hexyl-2-(4-t-butylphenyl)methyl-3,4-dihydro-6,7-dimethoxy isoquinolinium chloride (m.p. 34° C.).

¹H-NMR (CDCl₃, 300 MHz): δ 0.87 (t, 3H, J=7.6 Hz), 1.15˜1.40 (m, 13H), 1.40˜1.55 (m, 2H), 1.60˜1.75 (m, 2H), 3.20 (t, 2H, J=7.5 Hz), 2.30 (t, 2H, J=8.7 Hz), 3.95 (s, 3H), 4.01 (s, 3H), 4.19 (t, 2H, J=7.8 Hz), 5.56 (s, 2H), 6.86 (s, 1H), 7.27 (s, 1H), 7.31 (d, 2H, J=4.2 Hz), 7.44 (d, 2H, J=4.2 Hz)

### EXAMPLE 127

### Preparation of 1-undecyl-2-(4-t-butylphenyl)methyl-3,4-dihydro-6,7-dimethoxy isoquinolinium chloride (Compound No. 127)

Lauroyl chloride, instead of butyryl chloride of Example 1, was treated by the same process described in Example 1, to give 1.0 mmol of 1-undecyl-3,4-dihydro-6,7-dimethoxy isoquinoline. Then, the resulting product was reacted with 1.2 mmol of 4-t-butylbenzylchloride to give 420 mg of 1-undecyl-2-(4-t-butylphenyl)methyl-3,4-dihydro-6,7-dimethoxy isoquinolinium chloride (m.p. 85° C.).

¹H-NMR (CDCl₃,300 MHz): δ 0.87 (t, 3H, J=6.9 Hz), 1.31 (s, 9H), 1.00˜1.40 (m, 14H), 1.40˜1.50 (m, 2H), 1.60˜1.80 (m, 2H), 3.22 (t, 2H, J=7.8 Hz), 3.35 (t, 2H, J=7.8 Hz), 3.97 (s, 3H), 4.02 (s, 3H), 4.16 (t, 2H, J=7.2 Hz), 5.52 (s, 2H), 6.97 (s, 1H), 7.33 (d, 1H, J=8.7), 7.38 (s, 1H), 7.45 (d, 2H, J=8.7 Hz)

### EXAMPLE 128

### Preparation of 1-pentadecyl-2-(4-t-butylphenyl)methyl-3,4-dihydro-6,7-dimethoxy isoquinolinium chloride (Compound No. 128)

Palmitoyl chloride, instead of butyryl chloride of Example 1, was treated by the same process described in Example 1, to give 11.0 mmol of 1-pentadecyl-3,4-dihydro-6,7-dimethoxy isoquinoline. Then, the resulting product was reacted with 1.2 mmol of 4-t-butyl benzyl chloride to give 465 mg of 1-pentadecyl-2-(4-t-butylphenyl)methyl-3,4-dihydro-6,7-dimethoxyisoquinolinium chloride (m.p. 166° C.).

¹H-NMR (CDCl₃, 300 MHz): δ 0.88 (t, 3H, J=6.6 Hz), 1.00˜1.40 (m, 31H). 1.40˜1.55 (m, 2H), 1.55˜1.75 (m, 2H), 3.21 (t, 2H, J=6.9 Hz), 3.32 (t, 2H, J=7.8 Hz), 3.96 (s, 3H), 4.01 (s, 3H), 4.17 (t, 3H, J=5.55 (s, 2H), 6.93 (s, 1H), 7.30 (s, 1H), 7.32 (d, 2H, J=6.6 Hz), 7.44 (d, 2H, J=6.6 Hz)

### EXAMPLE 129

### Preparation of 1-(2-(4-t-butylphenyl))ethyl-2-(4-trifluoromethylphenyl)methyl-3,4-dihydro-6,7-dimethoxy isoquinolinium chloride (Compound No. 129)

4-(t-Butylphenyl)propionyl chloride, instead of butyryl chloride of Example 1, was treated by the same process described in Example 1, to give 1 mmol of 1-(2-(4-t-butylphenyl))ethyl-3,4-dihydro-6,7-dimethoxy isoquinoline. Then, the resulting product was reacted with 1.2 mmol of 4-trifluoromethylphenyl to give 470 mg of 1-(2-(4-t-butylphenyl))ethyl-2-(4-trifluoromethylphenyl)methyl-3,4-dihydro-6,7-dimethoxy isoquinolinium bromide (m.p. 77° C.).

¹H-NMR (CDCl₃, 300 MHz): δ 1.27 (s, 9H), 3.10 (t, 2H, J=7.2 Hz), 3.17 (t, 2H, J=7.2 Hz), 3.69 (t, 2H, J=7.8 Hz), 3.84 (s, 3H), 3.99 (s, 3H), 4.06 (t, 2H, J=7.8 Hz), 5.60 (s, 2H), 6.80 (s, 1H), 7.05 (d, 2H, J=8.4 Hz), 7.14 (s, 1H), 7.26 (d, 2H, J=8.4 Hz), 7.47 (d, 2H, J=7.8 Hz), 7.65 (d, 2H, J=7.8 Hz)

### EXAMPLE 130

### Preparation of 1-(2-(4-t-butylphenyl))ethyl-2-(2,3,4,5,6-pentafluorophenyl)methyl-3,4-dihydro-6,7-dimethoxy isoquinolinium chloride (Compound No. 130)

2,3,4,5,6-Pentafluorobenzyl bromide, instead of 4-trifluoromethylbenzyl chloride of Example 129, was treated by the same process described in Example 129, to give 488 mg of 1-(2-(4-t-butylphenyl))ethyl-2-(2,3,4,5,6-pentafluorophenyl)methyl-3,4-dihydro-6,7-dimethoxy isoquinolinium chloride

(m.p. 35° C.).

¹H-NMR (CDCl₃, 300 MHz): δ 1.26 (s, 9H), 3.10 (t, 2H, J=7.2 Hz), 3.25 (t, 2H, J=7.2 Hz), 3.70 (t, 2H, J=7.8 Hz), 3.81 (s, 3H), 3.99 (s, 3H), 4.10 (t, 2H, J=7.8 Hz), 5.70 (s, 2H), 6.80 (s, 1H), 7.10 (d, 2H, J=8.4 Hz), 7.14 (s, 1H), 7.26 (d, 2H, J=8.4 Hz)

### EXAMPLE 131

### Preparation of 1-(2-(4-t-butylphenyl))ethyl-2-(2,3,5,6-tetrafluoro-4-trifluoromethylphenyl)methyl-3,4-dihydro-6,7-dimethoxy isoquinolinium chloride (Compound No. 131)

2,3,5,6-Tetrafluoro-4-trifluoromethylbenzyl bromide, instead of 4-trifluoromethylbenzyl chloride of Example 129, was treated by the same process described in Example 129, to give 528 mg of 1-(2-(4-t-butylphenyl))ethyl-2-(2,3,5,6-tetrafluorophenyl-4-trifluoromethylphenyl)methyl-3,4-dihydro-6,7-dimethoxy isoquinolinium chloride (m.p. 38° C.).

¹H-NMR (CDCl₃, 300 MHz): δ 1.26 (s, 9H), 3.11 (t, 2H, J=6.9 Hz), 3.28 (t, 2H, J=6.9 Hz), 3.70 (t, 2H, J=7.5 Hz), 3.81 (s, 3H), 3.99 (s, 3H), 4.56 (t, 2H, J=7.5 Hz), 5.83 (s, 2H), 6.79 (s, 1H), 7.12 (d, 2H, J=8.4 Hz), 7.16 (s, 1H), 7.30 (d, 2H, J=8.4 Hz)

### EXAMPLE 132

### Preparation of 1-(2-(4-t-butylphenyl))ethyl-2-(2-(4-trifluoromethylbenzyloxy)-3-methoxyphenyl)methyl-3,4-dihydro-6,7-dimethoxy isoquinolinium chloride (Compound No. 132)

2-(4-Trifluoromethylbenzyloxy)-3-methoxybenzyl chloride, instead of 4-trifluoromethylbenzyl chloride of Example 129, was treated by the same process described in Example 129, to give 544 mg of 1-(2-(4-t-butylphenyl))ethyl-2-(2-(4-trifluoromethylbenzyloxy)-3-methoxyphenyl)methyl-3,4-dihydro-6,7-dimethoxyisoquinolinium chloride (m.p. 60° C.).

¹H-NMR (CDCl₃, 300 MHz): δ 1.27 (s, 9H), 2.86 (t, 2H, J=7.5 Hz), 3.08 (t, 2H, J=7.5 Hz), 3.31 (t, 2H, J=8.1 Hz), 3.78 (s, 3H), 3.90 (s, 3H), 3.98 (s, 3H), 4.04 (t, 2H, J=8.1 Hz), 5.23 (s, 2H), 5.53 (s, 2H), 6.78 (s, 1H), 6.87 (s, 1H), 6.97 (d, 2H, J=8.4 Hz), 7.0˜7.15 (m, 1H) 7.25˜7.45 (m, 2H), 7.22 (d, 2H, J=8.4 Hz), 7.53 (d, 2H, J=8.7 Hz), 7.56 (d, 2H, J=8.7 Hz)

### EXAMPLE 133

### Preparation of 1-(2-(4-t-butylphenyl))ethyl-2-(2-(2-chloro-6-fluorobenzyloxy)-3-methoxyphenyl)methyl-3,4-dihydro-6,7-dimethoxyisoquinolinium chloride (Compound No. 133)

2-(2-Chloro-4-fluorobenzyloxy)-3-methoxybenzyl chloride, instead of 4-trifluoromethylbenzyl chloride of Example 129, was treated by the same process described in Example 129, to give 531 mg of 1-(2-(4-t-butylphenyl)) ethyl-2-(2-(2-chloro-6-fluorobenzyloxy)-3-methoxyphenyl)methyl-3,4-dihydro-6,7-dimethoxy isoquinolinium chloride (m.p. 63° C.).

¹H-NMR (CDCl₃, 300 MHz): δ 1.28 (s, 9H), 2.75 (t, 2H, J=7.5 Hz), 3.04 (t, 2H, J=7.5 Hz), 3.36 (t, 2H, J=7.2 Hz), 3.81 (s, 3H), 3.93 (s, 3H), 3.95 (t, 3H, J=7.2 Hz), 3.97 (s, 3H), 5.36 (s, 2H), 5.41 (d, 2H, J=2.1 Hz), 6.78 (s, 1H), 6.96 (d, 2H, J=8.7 Hz), 7.24 (d, 2H, J=8.7 Hz), 3.9˜7.1 (m, 3H), 7.10˜7.30 (m, 4H)

### EXAMPLE 134

### Preparation of 1-(2-(4-t-butylphenyl))ethyl-2-(2-octyloxy-3-methoxyphenyl)methyl-3,4-dihydro-6,7-dimethoxy isoquinolinium chloride (Compound No. 134)

2-Octyloxy-3-methoxybenzyl chloride, instead of 4-trifluoromethyl benzyl chloride of Example 129, was treated by the same process described in Example 129, to give 526 mg of 1-(2-(4-t-butylphenyl))ethyl-2-(2-octyloxy-3-methoxyphenyl)methyl-3,4-dihydro-6,7-dimethoxy isoquinolinium chloride (m.p. 76° C.).

¹H-NMR (CDCl₃, 300 MHz): δ 0.65 (t, 3H, J=6.9 Hz), 1.03 (s, 9H), 1.00˜1.40 (m, 12H), 2.79 (t, 3H, 6.9 Hz), 3.53 (t, 2H, J=6.9 Hz), 3.65 (s, 3H), 3.60 (s, 3H), 3.70 (t, 2H, J=7.2 Hz), 3.78 (t, 2H, J=6.9 Hz), 3.89 (s, 3H), 4.05 (t, 2H, J=7.2 Hz), 5.04 (s, 2H), 6.80 (s, 1H), 6.99 (d, 1H, J=7.8 Hz), 7.00˜7.10 (m, 3H), 7.14 (d, 2H, J=7.8 Hz), 7.19 (s, 1H), 7.20˜7.30 (m, 2H), 7.29 (d, 2H, J=7.8 Hz)

### EXAMPLE 135

### Preparation of 1-(2-(4-t-butylphenyl))ethyl-2-(2-(2,3,5,6-tetrafluoro-4-trifluoromethylbenzyloxy)-3-methoxyphenyl)methyl-3,4-dihydro-6,7-dimethoxy isoquinolinium chloride (Compound No. 135)

2-(2,3,5,6-Tetrafluoro-4-trifluoromethylbenzyloxy)-3-methoxy benzyl chloride, instead of 4-trifluoromethylbenzyl chloride of Example 129, was treated by the same process described in Example 129, to give 639 mg of 1-(2-(4-t-butylphenyl))ethyl-2-(2-(2,3,5,6-tetrafluoro-4-trifluoromethylbenzyloxy)-3-methoxyphenyl)methyl-3,4-dihydro-6,7-dimethoxy isoquinolinium chloride (m.p. 140° C.).

¹H-NMR (CDCl₃, 300 MHz): δ 1.26 (s, 9H), 2.99 (t, 2H, J=7.8 Hz), 3.11 (t, 2H, J=7.8 Hz), 3.70 (t, 2H, J=7.2 Hz), 3.84 (s, 3H), 3.86 (s, 3H), 3.98 (s, 3H), 4.05 (t, 2H, J=7.2 Hz), 5.41 (s, 2H), 5.59 (s, 2H), 6.81 (s, 1H), 6.98˜7.04 (m, 1H), 7.04 (d, 2H, J=5.7 Hz), 7.10 (s, 1H), 7.16 (d, 2H, J=5.7 Hz), 7.22˜7.30 (m, 2H)

### EXAMPLE 136

### Preparation of 1-(2-(4-t-butylphenyl))ethyl-2-(2-(2,3-dimethoxybenzyloxy)-3-methoxyphenyl)methyl-3,4-dihydro-6,7-dimethoxy isoquinolinium chloride (Compound No. 136)

2-(2,3-Dimethoxybenzyloxy)-3-methoxybenzyl chloride, instead of 4-trifluoromethylbenzyl chloride of Example 129, was treated by the same process described in Example 129, to give 552 mg of 1-(2-(4-t-butylphenyl))ethyl-2-(2-(2,3-dimethoxybenzyloxy)-3-methoxyphenyl)methyl-3,4-dihydro-6,7-dimethoxy isoquinolinium chloride (m.p. 157° C.).

¹H-NMR (CDCl₃, 300 MHz): δ 1.28 (s, 9H), 3.03 (t, 2H, J=7.5 Hz), 3.16 (t, 2H, J=7.5 Hz), 3.73 (t, 2H, J=7.8 Hz), 3.85 (s, 3H), 3.88 (s, 3H), 3.89 (s, 3H), 3.90 (s, 3H), 3.98 (s, 3H), 4.12 (t, 2H, J=7.8 Hz), 5.48 (s, 2H), 6.80 (s, 1H), 6.99 (d, 1H, J=7.8 Hz), 7.06˜7.16 (m, 3H), 7.10 (d, 2H, J=7.8 Hz), 7.18˜7.26 (m, 2H), 7.26 (s, 1H), 7.29 (d, 2H, J=7.8 Hz)

### EXAMPLE 137

### Preparation of 1-undecyl-2-(4-trifluoromethylphenyl)methyl-3,4-dihydro-6-fluoro isoquinolinium chloride (Compound No. 137)

Lauroyl chloride, instead of butyryl chloride of Example 1, was treated by the same process described in Example 1, to give 11.0 mmol of 1-undecyl-3,4-dihydro-6,7-dimethoxyisoquinoline. Then, the resulting mixture was reacted with 4-trifluoromethylmethylbenzyl chloride to give 450 mg of 1-undecyl-2-(4-trifluoromethylphenyl)methyl-3,4-dihydro-6,7-dimethoxyisoquinolinium chloride (m.p. 122° C.).

¹H-NMR (CDCl₃, 300 MHz): δ 0.88 (t, 3H, J=6.9 Hz), 1.15˜1.35 (m, 3H), 1.45˜1.55 (m, 3H), 1.60˜1.80 (m, 4H), 3.34 (t, 2H, J=7.2), 3.67 (t, 2H, J=8.4 Hz), 4.20 (t, 2H, J=7.2 Hz), 5.81 (s, 2H), 7.10 (dd, 1H, J=8.4 Hz, J=2.4 Hz), 7.18˜7.28 (m, 1H), 7.58 (d, 2H, J=8.4 Hz), 7.72 (d, 2H, J=8.4 Hz), 7.94˜8.02 (m, 1H)

### EXAMPLE 138

### Preparation of 1-methyl-2-(2-(4-t-butylbenzyloxy)-3-methoxyphenyl)methyl-3,4-dihydro-6,7-dimethoxy isoquinolinium chloride (Compound No. 138)

Acetyl chloride, instead of butyryl chloride of Example 1, was treated by the same process described in Example 1, to give 1.0 mmol of 1-methyl-3,4-dihydro-6,7-dimethoxyisoquinoline. Then, the resulting mixture was reacted with 1.2 mmol of 2-(4-t-butylbenzyloxy)-3-methoxy benzylchloride to give 420 mg of 1-methyl-2-(2-(4-t-butylbenzyloxy)-3-methoxyphenyl)methyl-3,4-dihydro-6,7-dimethoxy isoquinolinium chloride (m.p. 105° C.).

¹H-NMR (CDCl₃, 300 MHz): δ 1.02 (s, 9H), 3.22 (t, 2H, J=7.8 Hz), 3.35 (t, 2H, J=7.8 Hz), 3.40 (s, 3H), 3.89 (s, 3H), 3.90 (s, 3H), 4.10 (s, 3H), 6.30 (s, 2H), 6.20 (s, 2H), 6.97 (s, 1H), 7.00˜7.20 (m, 3H), 7.33 (d, 2H, J=8.7 Hz), 7.38 (s, 1H), 7.45 (d, 2H, J=8.7 Hz).

### EXAMPLE 139

### Preparation of 1-propyl-2-(2-chloro-6-fluorophenyl)methyl-6,7-dimethoxy-isoquinolinium chloride (Compound No. 139)

were added To a 250 ml of dichloromethane solution containing 3,4-dimethoxyphenethylamine was added 12.14 g of triethylamine and 11.7 g of butyryl chloride at 0° C., and stirred for 2-3 hours and then warmed to room temperature. The reaction mixture was washed with 250 ml of thin hydrochloric acid solution and separated into organic phase and aqueous phase. The organic phase thus separated was dried and filtered and concentrated. The reaction mixture was separated through silica-gel column chromatography eluting with hexane and ethylacetate (1:3), to give 90% more of 3,4-dimethoxy phenethyl propionylamide.

The amide thus obtained was dissolved in 250 ml of acetonitrile, and 12.7 ml of phosphoryl chloride was added thereto, and the reaction mixture was heated for 4 hours under reflux and concentrated under reduced pressure. The resulting mixture was neutralized with saturated sodium carbonate solution and extracted with dichloromethane therefrom. The concentrated reaction mixture thus obtained was separated through silica-gel column chromatography eluting with dichloromethane and methanol (20:1), to give 18.9 g of 1-propyl-3,4-dihydro-6,7-dimethoxyisoquinoline (yield: 90%)

2.33 g of 1-propyl-3,4-dihydro-6,7-dimethoxyisoquinoline thus obtained was dissolved in 40 ml of tetrahydrofuran and 2.2 g of potassium t-butoxide was added thereto. The resulting mixture was heated to proceed the reaction for 24 hours at reflux temperature.

The resulting mixture was cooled to room temperature and washed with water then extracted with ethyl acetate. The concentrated reaction mixture thus obtained was separated through silica-gel column chromatography eluting with dichloromethane and methanol (40:1), to give 2.08 g of 6,7-dimethoxyisoquinoline (yield: 90%) 231 Mg of 6,7-dimethoxyisoquinoline thus obtained was dissolved in 10 ml of acetonitrile and 214 mg of 2′-chloro-6′-fluorobenzyl chloride was added thereto to proceed the reaction for 12 hours. The reaction mixture was cooled to room temperature and the solvent was removed from the reaction mixture under reduced pressure. The concentrated reaction mixture was separated through silica-gel column chromatography eluting with dichloromethane and methanol (10:1), to give 350 mg of 1-propyl-2-(2-chloro-6-fluorophenyl)methyl-6,7-dimethoxy-isoquinolinium chloride (yield: 85%) (m.p. 78° C.).

¹H-NMR (CDCl₃, 300 MHz): δ 1.16 (t, 3H, J=7.5 Hz), 1.31 (s, 9H), 1.6˜1.8 (m, 2H), 3.52 (t, 2H, J=7.5), 4.13 (s, 3H), 4.20 (s, 3H), 6.27 (s, 2H), 7.15 (d, 2H, J=6.9 Hz), 7.35 (d, 2H, J=6.9 Hz), 7.40 (s, 1H), 7.60 (s, 1H), 8.34 (d, 1H, J=6.9 Hz), 8.17 (d, 1H, J=6.9 Hz)

### EXAMPLE 140

### Preparation of 1-methyl-2-(2-chloro-6-fluorophenyl)methyl-6,7-dimethoxyisoquinolinium chloride (Compound No. 140)

Acetic anhydride, instead of butyryl chloride of Example 139, was treated by the same process described in Example 139, to give 203 mg of 1-methyl-6,7-dimethoxyisoquinoline. The resulting mixture was dissolved in 10 ml of acetonitrile and 214 mg of 2′-chloro-6′-fluorobenzyl chloride was added thereto to proceed the reaction for 12 hours at reflux temperature. The reaction mixture was cooled to room temperature and the solvent was removed from the reaction mixture under reduced pressure. The concentrated reaction mixture was separated through silica-gel column chromatography eluting with dichloromethane and methanol (10:1), to give 320 mg of 1-methyl-2-(2-chloro-6-fluorophenyl)methyl-6,7-dimethoxyisoquinolinium chloride (m.p. 96° C.).

¹H-NMR (CDCl₃, 300 MHz): δ 3.39 (s, 3H), 4.15 (s, 3H), 4.16 (s, 3H), 6.30 (s, 2H), 7.10˜7.15 (m, 1H), 7.25˜7.35 (m, 1H), 7.35˜7.45 (m, 1H), 7.64 (s, 1H), 7.66 (s, 1H), 8.24 (d, 1H, J=7.2 Hz), 8.48 (dd, 1H, J=7.2 Hz, 1.5 Hz)

### EXAMPLE 141

### Preparation of 2-(2-chloro-6-fluorophenyl)methyl-6,7-dimethoxyisoquinolinium chloride (Compound No. 141)

Methyl formate, instead of butyryl chloride of Example 139, was treated by the same process described in Example 139, to give 189 mg of 6,7-dimethoxyisoquinoline. The resulting mixture was dissolved in 10 ml of acetonitrile and 214 mg of 2-chloro-6-fluorobenzyl chloride was added thereto to proceed the reaction for 12 hours at reflux temperature. The reaction mixture was cooled to room temperature and the solvent was removed from the reaction mixture under reduced pressure. The concentrated reaction mixture was separated through silica-gel column chromatography eluting with dichloromethane and methanol (10:1), to give 312 mg of 2-(2-chloro-6-fluorophenyl)methyl-6,7-dimethoxy-isoquinolinium chloride (m.p. 147° C.).

¹H-NMR (CDCl₃, 300 MHz): δ 4.06 (s, 3H), 4.13 (s, 3H), 6.08 (s, 2H), 7.31˜7.37 (m, 1H), 7.47 (d, 1H, J=8.7 Hz), 7.54˜7.59)m, 1H), 7.67 (s, 1H), 7.82 (s, 1H), 9.51 (s, 1H)

### EXAMPLE 142

### Preparation of 2-(4-t-butylphenyl)methyl-6,7-dimethoxy-isoquinolinium chloride (Compound No. 142)

4-t-Butylbenzyl chloride, instead of 2-chloro-6-fluorobenzyl chloride obtained from Example 139, was treated by the same process described in Example 139, to give 342 mg of 2-(4-t-butylphenyl)methyl-6,7-dimethoxyisoquinolinium chloride (yield: 92%) (m.p. 47° C.).

¹H-NMR (CDCl₃, 300 MHz): δ 1.25 (s, 9H), 4.08 (s, 3H), 4.11 (s, 3H), 6.07 (s, 2H), 7.33 (s, 1H), 7.36 (d, 2H, J=8.4 Hz), 7.56 (d, 2H, J=8.4 Hz), 8.00 (d, 1H, J=6.9 Hz), 8.01 (s, 1H), 8.38 (d, 1H, J=6.9 Hz), 10.80 (s, 1H)

### EXAMPLE 143

### Preparation of 1-methyl-2-(4-t-butylphenyl)methyl-6,7-dimethoxyisoquinolinium chloride (Compound No. 143)

203 Mg of 1-methyl-6,7-dimethoxyisoquinoline obtained from Example 139 was reacted with 4-t-butylbenzyl chloride to give 341 mg of 1-methyl-2-(4-t-butylphenyl)methyl-6,7-dimethoxyisoquinolinium chloride (m.p. 350° C.).

¹H-NMR (CDCl₃, 300 MHz): δ1.27 (s, 9H), 3.27 (s, 3H), 4.11 (s, 3H), 4.45 (s, 3H), 6.25 (s, 2H), 7.15 (d, 2H, J=7.8 Hz), 7.35 (d, 2H, J=7.8 Hz), 7.53 (s, 1H), 7.56 (s, 1H), 8.24 (d, 1H, J=6.3 Hz), 8.88 (d, 1H, J=6.3 Hz)

### EXAMPLE 144

### Preparation of 1-propyl-2-(4-t-butylphenyl)methyl-6,7-dimethoxyisoquinolinium chloride (Compound No. 144)

1.0 Mmol of 1-propyl-6,7-dimethoxyisoquinoline obtained from Example 139 was reacted with 1.2 mmol of 4-t-butylbenzyl chloride to give 353 mg of 1-propyl-2-(4-t-butylphenyl)methyl-6,7-dimethoxyisoquinolinium chloride (m.p. 79° C.).

¹H-NMR (CDCl₃, 300 MHz): δ 1.16 (t, 3H, J=7.6 Hz), 1.20˜1.40 (s, 9H), 1.60˜1.8 (m, 2H), 3.50 (t, 2H, J=7.4 Hz, 4.13 (s, 3H), 4.20 (s, 3H), 6.27 (s, 2H), 7.15 (d, 2H, J=8.2 Hz), 7.35 (d, 2H, J=8.2 Hz), 7.40 (s, 1H), 7.60 (s, 1H), 8.34 (d, 1H, J=6.9 Hz), 9.17 (d, 1H, J=6.9 Hz)

### EXAMPLE 145

### Preparation of 1-hexyl-2-(4-t-butylphenyl)methyl-6,7-dimethoxyisoquinolinium chloride (Compound No. 145)

Heptanoyl chloride, instead of butyryl chloride of Example 139, was treated by the same process described in Example 139, to give 1.0 mmol of 1-hexyl-6,7-dimethoxyisoquinoline. Then, the resulting product was reacted with 1.2 mmol of 4-t-butylbenzyl chloride to give 346 mg of 1-hexyl-2-(4-t-butylphenyl)methyl-6,7-dimethoxyisoquinolinium chloride (m.p. 105° C.).

¹H-NMR (CDCl₃, 300 MHz): δ 0.87 (t, 3H, J=6.9 Hz), 1.0˜1.4 (m, 4H), 1.262 (s, 9H), 1.40˜1.60 (m, 4H), 3.40˜3.60 (m, 2H), 4.08 (s, 3H), 4.13 (s, 3H), 6.20 (s, 2H), 7.15 (d, 2H, J=8.4 Hz), 7.31 (d, 2H, J=8.4 Hz), 7.42 (s, 1H), 7.69 (s, 1H), 8.37 (d, 1H, J=6.6 Hz), 9.04 (d, 1H, J=6.6 Hz)

### EXAMPLE 146

### Preparation of 1-undecyl-2-(4-t-butylphenyl)methyl-6,7-dimethoxyisoquinolinium chloride (Compound No. 146)

Lauroyl chloride, instead of butyryl chloride of Example 139, was treated by the same process described in Example 139, to give 1.0 mmol of 1-undecyl-6,7-dimethoxyisoquinoline. Then, the resulting product was reacted with 1.2 mmol of benzyl chloride to give 420 mg of 1-undecyl-2-(4-t-butyl-phenyl)methyl-6,7-dimethoxyisoquinolinium chloride (m.p. 91° C.).

¹H-NMR (CDCl₃, 300 MHz): δ 0.91 (t, 3H, J=6.0 Hz), 1.00˜4.40 (m, 23H), 1.40˜1.60 (m, 4H), 3.40˜3.55 (bs, 2H), 4.12 (s, 3H), 4.19 (s, 3H), 6.26 (s, 2H), 7.18 (d, 2H, J=4.2 Hz), 7.38 (d, 2H, J=4.2 Hz), 7.46 (s, 1H), 7.77 (s, 1H), 8.42 (d, 1H, J=6.3 Hz), 9.11 (d, 1H, J=6.3 Hz)

### EXAMPLE 147

### Preparation of 1-pentadecyl-2-(4-t-butylphenyl)methyl-6,7-dimethoxyisoquinolinium chloride (Compound No. 147)

Palmitoyl chloride, instead of butyryl chloride of Example 139, was treated by the same process described in Example 139, to give 11.0 mmol of 1-pentadecyl-6,7-dimethoxyisoquinoline. Then, the resulting mixture was reacted with 1.2 mmol of 4-t-butylbenzyl chloride to give 483 mg of 1-pentadecyl-2-(4-t-butylphenyl)methyl-6,7-dimethoxyisoquinolinium chloride.

(m.p. 120° C.).

¹H-NMR (CDCl₃, 300 MHz): δ 0.89 (3H, t, J=6.9 Hz), 1.00˜1.40 (m, 31H), 1.40˜1.60 (m, 4H), 3.4˜3.6 (bs, 2H), 4.12 (s, 3H), 4.17 (s, 3H), 6.25 (s, 2H), 7.19 (d, 2H, J=8.1 Hz), 7.35 (d, 2H, J=8.1 Hz), 7.49 (s, 1H), 7.81 (s, 1H), 8.46 (d, 1H, J=4.5 Hz), 9.00 (d, 1H, J=4.5 Hz)

### EXAMPLE 148

### Preparation of 1-(2-(4-t-butylphenyl))ethyl-2-(2-octyloxy)-3-dimethoxyphenyl)methyl-6,7-dimethoxyisoquinolinium chloride (Compound No. 148)

2-Octyloxy-3-methoxybenzyl chloride, instead of 2-(2,3-dimethoxybenzyloxy)-3-methoxybenzyl chloride of Example 139, was treated by the same process described in Example 139, to give 526 mg of 1-(2-(4-t-butylphenyl)) ethyl-2-(2-octyloxy)-3-dimethoxyphenyl)methyl-6,7-dimethoxyisoquinolinium chloride (m.p. 143° C.).

¹H-NMR (CDCl₃, 300 MHz): δ 0.61 (t, 3H, J=6.9 Hz), 1.03 (s, 9H), 1.00˜1.40 (m, 12H), 2.78 (t, 3H, J=6.9 Hz), 3.53 (t, 2H, J=6.9 Hz), 3.60 (s, 3H), 3.65 (s, 3H), 3.78 (t, 2H, J=6.9 Hz), 3.89 (s, 3H), 5.86 (s, 2H), 6.41 (dd, 1H, J=7.5 Hz, J=1.2 Hz), 7.01 (d, 2H, J=8.1 Hz), 6.75˜6.90 (m, 2H), 6.70 (d, 2H, J=8.1 Hz), 7.31 (s, 1H), 8.14 (d, 1H, J=6.6 Hz), 9.04 (d, 1H, J=6.6 Hz)

### EXAMPLE 149

### Preparation of 1-(2-(4-t-butylphenyl))ethyl-2-(2-(4-trifluoromethylbenzyloxy)-3-methoxyphenyl)methyl-6,7-dimethoxyisoquinolinium chloride (Compound No. 149)

2-(4-Trifluoromethylbenzyloxy)-3-methoxybenzyl chloride, instead of 2-(2,3-dimethoxybenzyloxy)-3-methoxybenzyl chloride of Example 139, was treated by the same process described in Example 139, to give 578 mg of 1-(2-(4-t-butylphenyl))ethyl-2-(2-(4-trifluoromethylbenzyloxy)-3-methoxyphenyl)methyl-6,7-dimethoxyisoquinolinium chloride (m.p. 110° C.).

¹H-NMR (CDCl₃, 300 MHz): δ 1.27 (s, 9H), 2.80 (t, 2H, J=7.5 Hz), 3.63 (t, 2H, J=7.5 Hz), 3.89 (s, 3H), 3.90 (s, 3H), 4.15 (s, 3H), 5.17 (s, 2H), 6.22 (s, 2H), 6.68 (dd, 1H, J=7.5 Hz, J=0.9 Hz), 6.87 (d, 2H, J=8.4 Hz), 6.97 (d, 1H, J=7.5 Hz), 7.06 (d, 1H, J=11.4 Hz), 7.02˜7.10 (m, 1H), 7.21 (d, 2H, J=7.5 Hz), 7.50˜7.62 (m, 5H), 8.27 (d, 1H, J=6.9 Hz), 9.19 (d, 1H, J=6.9 Hz)

### EXAMPLE 150

### Preparation of 1-(2-(4-t-butylphenyl))ethyl-2-(2-(2-chloro-6-fluorobenzyloxy)-3-methoxyphenyl)methyl-6,7-dimethoxyisoquinolinium chloride (Compound No. 150)

2-(2-Chloro-4-fluorobenzyloxy)-3-methoxybenzyl chloride, instead of 2-(2,3-dimethoxybenzyloxy)-3-methoxybenzyl chloride of Example 139, was treated by the same process described in Example 139, to give 424 mg of 1-(2-(4-t-butylphenyl))ethyl-2-(2-(2-chloro-6-fluorobenzyloxy)-3-methoxyphenyl)methyl-6,7-dimethoxyisoquinolinium chloride (m.p. 60° C.).

¹H-NMR (CDCl₃, 300 MHz): δ 1.27 (s, 9H), 2.83 (t, 2H, J=6.9 Hz), 3.66 (t, 2H, J=6.9 Hz), 3.86 (s, 3H), 3.94 (s, 3H), 4.14 (s, 2H), 5.91 (s, 1H), 6.88 (d, 2H, J=8.1 Hz), 7.21 (d, 2H, J=8.1 Hz), 6.90˜7.10 (m, 2H), 7.20˜7.40 (m, 1H), 7.67 (s, 1H), 8.49 (d, 1H, J=4.8 Hz), 8.92 (d, 1H, J=4.8 Hz)

### EXAMPLE 151

### Preparation of 1-(2-(4-t-butylphenyl))ethyl-2-(2-(2,3,5,6-tetrafluoro-4-trifluoromethylbenzyloxy)-3-methoxyphenyl)methyl-6,7-dimethoxyisoquinolinium chloride (Compound No. 151)

2-(2,3,5,6-Tetrafluoro-4-trifluoromethylenebenzyloxy)-3-methoxy benzyl chloride, instead of 2-(2,3-dimethoxybenzyloxy)-3-methoxy benzyl chloride of Example 139, was treated by the same process described in Example 139, to give 500 mg of 1-(2-(4-t-butylphenyl))ethyl-2-(2-(2,3,5,6-tetrafluoro-4-trifluoromethylbenzyloxy)-3-methoxyphenyl)methyl-6,7-dimethoxyisoquinolinium chloride (m.p. 72° C.).

¹H-NMR (CDCl₃, 300 MHz): δ 1.27 (s, 9H), 2.87 (t. 2H, J=6.9 Hz), 3.71 (t, 2H, J=6.9 Hz), 3.88 (s, 3H), 3.89 (s, 3H), 4.16 (s, 2H), 6.40 (s, 1H), 6.91 (d, 2H, J=8.4 Hz), 7.23 (d, 2H, J=8.4 Hz), 7.43 (s, 1H)

### EXAMPLE 152

### Antifungal Efficacy of Cream Formulation Against Local Fungal Skin Infection

SPF (Specific Pathogen Free) SKH/1 male mouse (hairless) of which weight is 30-35 g and age is 5 weeks, was reared with sterilized water and feed for 12 night/day in the condition of 21-23° C. and of 50% of relative humidity. Five mice were allocated by each group. After skin infection, each mouse was taken in separate cage.

Epidermophyton floccosum was cultivated on a flat medium of SDA (Sabouraud Dextrose Agar) for 5-7 days and after confirmation of macrocornidia, 3 ml of PRMI (Rosewell Park Memorial Institute) 1640 media per each flat medium was added thereto and then scraped well using loop to remove hyphae from media. The floating liquid was suspended briefly and then diluted with PRMI 1640 media to adjust the concentration of hyphae to 2×106 CFU/ml. Mouse was anesthetized by ethyl ester and marked on back site (lumbosacral area) in a shape of circle of which diameter is 1.5 cm. Then, the inner part of marked skin was scratched with sand paper. The scratched part was covered with a filter paper to preserve inoculated microorganism for a long time and thereby stimulating the skin continuously. 0.2 ml of fungus solution of which concentration is adjusted as described above, was inoculated between the skin and filter paper.

Days after the inoculation, the filter paper was removed and the infection of skin was examined. The test formulations, 0.5% creamy formulations of the compounds No. 12 and 0.5% creamy formulations of the compounds No. 25, 1.0% creamy formulation of Terbinafine (Lamisil cream and a placebo were applied on the infected areas in the same amount once a day for 5 days. The clinical evaluation on the change of the infected area in 5 days after the inoculation was performed and expressed numerically from 0 to 4. The daily change of the infected area was checked every day. The result of each group was compared with each other.

0: Normal state

1: Mild erythema or small number of skin eruption

2: Well-demarcated erythema with scales or mild skin eruption of infected area

3: Wide area of marked skin eruption, scales, swelling or severe skin eruption with partial swelling and scales

4: The same as those of control, or severe skin eruption in entire lesion

T: Average score of clinical evacuation in drug treated area

The result was calculated as follows;

Efficacy (%)=100−(T×100)/K)

(K: Average score of clinical evaluation in placebo control group)

### EXAMPLE 153

### Preparation of Pharmaceutically Available Tablets of Isoquinoline Salt derivatives

The raw drug materials corresponding to an amount of 10,000 tablets were weighted and passed into 20 mesh sieve and the mixture was then blended for 10 minutes. The mixture was transferred to a compressor and was tableted under suitable pressure so as to give average weight of 200 mg per tablet.

1) Composition of the raw drug materials per tablet (200 mg):

Component amount

Compound 12 10 mg

Calcium carboxymethylcellulose 5 mg

Lactose #100 (100 mesh) 147.5 mg

Hydroxypropylcellulose 5 mg

Ludipress (BASF AG) 30 mg

Magnesium stearate 2.5 mg

2) Composition of the raw drug materials per tablet (200 mg):

Component amount

Compound No. 119 10 mg

Calcium carboxymethylcellulose 5 mg

Lactose #100 (100 mesh) 147.5 mg

Hydroxypropylcellulose 5 mg

Kollidon VA64 (BASF AG) 30 mg

Magnesium stearate 2.5 mg

3) Composition of the raw drug materials per tablet (200 mg):

Component amount

Compound No. 134 5 mg

Calcium carboxylmethylcellulose 5 mg

Lactose #100 (100 mesh) 152.5 mg

Hydroxypropylcellulose 5 mg

Ludipress (BASF AG) 30 mg

Magnesium stearate 2.5 mg

4) Composition of the raw drug materials per tablet (200 mg):

Component amount

Compound No. 148 5 mg

Calcium carboxylmethylcellulose 5 mg

Lactose #100 (100 mesh) 152.5 mg

Hydroxypropylcellulose 5 mg

Kollidon VA64 (BASF AG) 30 mg

Magnesium sterate 2.5 mg

5) Composition of the raw drug materials per tablet (200 mg):

Component amount

Compound No. 149 2 mg

Calcium carboxylmethylcellulose 5 mg

Lactose #100 (100 mesh) 155.5 mg

Hydroxypropylcellulose 5 mg

Ludipress (BASF AG) 30 mg

Magnesium stearate 2.5 mg

6) Composition of the raw drug materials per tablet (200 mg):

Component amount

Compound No. 150 2 mg

Calcium carboxylmethylcellulose 5 mg

Lactose #100 (100 mesh) 155.5 mg

Hydroxypropylcellulose 5 mg

Kollidon VA64 (BASF AG) 30 mg

Magnesium sterate 2.5 mg

### EXAMPLE 154

### Preparation of 0.5% Creamy Formulation of Compound No. 12

Tefose 63 (80 g) produced by GATTEFOSSE (France), 15.32 g of Labaril M 1944 CS and 14.4 g of liquid paraffine were heated to 70° C. and 2.0 g of the compound No. 12 was added thereto and then suspended with stirring (8,000 rpm) for 10 minutes. The suspension thus obtained was added to water solution at 70° C. wherein 2.0 g of disodium hydrogen phosphate (Na2HPO4) was dissolved in 300 g of purified water, and emulsified with stirring (8,000 rpm) for 20 minutes. The emulsion thus obtained was cooled to 35° C. with stirring and charged in tube by suitable amount.

### EXAMPLE 155

### Preparation of 0.5% Creamy Formulation of Compound No. 119

Tefose 63 (80 g) produced by GATTEFOSSE (France), 15.32 g of Labaril M 1944 CS and 14.4 g of liquid paraffine were heated to 70° C. and 2.0 g of the compound No. 12 was added thereto and then suspended with stirring (8,000 rpm) for 10 minutes. The suspension thus obtained was added to water solution at 70° C. wherein 2.0 g of disodium hydrogen phosphate (Na2HPO4) was dissolved in 300 g of purified water, and emulsified with stirring (8,000 rpm) for 20 minutes. The emulsion thus obtained was cooled to 35° C. with stirring and charged in tube by suitable amount.

### EXAMPLE 156

### Preparation of Vaginal Suppository of Compound No. 12

The compound No. 12 (10 g), 50 g of succinic acid, 100 g of potassium sulphate, 20 g of silicon dioxide (SiO2) and 180 g of lactose #100 (100 Mesh) were mixed in mixer for 5 minutes, and 8,560 g of lactose #100 (100 Mesh) and 1,000 g of Ludipress were added thereto and then mixed for 10 minutes. Magnesium stearate (80 g) was added to the mixture and further mixed for 5 minutes. The resulting mixture was tableted using a punch to prepare 10,000 tablets of which thickness is 6.0 mm and weight is 1,000 mg (Hardness: 8 KP, Friction loss: 0.2%, Disintegration rate: 120 seconds).

### EXAMPLE 157

### Preparation of Vaginal Suppository of Compound No. 119

The compound No. 12 (10 g), 50 g of succinic acid, 100 g of potassium sulphate, 20 g of silicon dioxide (SiO2) and 180 g of lactose #100 (100 Mesh) were mixed in mixer for minutes, and 8,560 g of lactose #1.0 (100 Mesh) and 1,000 g of Ludipress were added thereto and then mixed for 10 minutes. Magnesium stearate (80 g) was added to the mixture and further mixed for 5 minutes. The resulting mixture was tableted using a punch to prepare 10,000 tablets of which thickness is 6.0 mm and weight is 1,000 mg (Hardness: 8 KP, Friction loss: 0.2%, Disintegration rate: 110 seconds).

### ADVANTAGEOUS EFFECTS

3,4-Dihydroisoquinolinium salt derivatives and isoquinolinium salt derivatives of the above Table 1 can effectively inhibit a Chitin synthetase which take part in biosynthesis of a component of cell wall, Chitin and 24-methyl transferase which is one of main enzyme for distal biosynthetic pathway of a component of the cell membrane, Ergosterol and thus, are effective in treating fungal infections.

The MIC (Minimal Inhibitory Concentration), data of the compound Nos. 12, 119, 120, 121, 127, 132, 134, 135, 148, 149, 150, 151 of the above Table. 1 and various kinds of Candida such as, Miconazole of azole compounds and Amphotericin B of polyene compounds, are described in the following Table 11.

TABLE 11
MIC data of the compounds

| | Compound | Compound | Compound | Compound | Compound | Compound | Compound |
|---|---|---|---|---|---|---|---|
| Fungus | No. 12 | No. 119 | No. 120 | No. 121 | No. 127 | No. 132 | No. 134 |
| C. albicnas ATCC | 1.56 | 1.56 | 3.125 | 3.125 | 1.56 | 1.56 | 1.56 |
| 10231 | | | | | | | |
| C. albicans IFO 1385 | 3.125 | 3.125 | 3.125 | 1.56 | 0.78 | 6.25 | 3.125 |
| C. albicans ATCC | 1.56 | 1.56 | 1.56 | 3.125 | 0.78 | 3.125 | 3.125 |
| 11651 | | | | | | | |
| C. albicans ATCC | 1.56 | 1.56 | 1.56 | 0.78 | 1.56 | 1.56 | 1.56 |
| 28838 | | | | | | | |
| C. albicans OY-003 | 1.56 | 1.56 | 1.56 | 3.125 | 1.56 | 3.125 | 1.56 |
| C. albicans OY-019 | 3.125 | 3.125 | 3.125 | 3.125 | 0.78 | 3.125 | 1.56 |
| C. albicans U.K | 1.56 | 1.56 | 1.56 | 3.125 | 1.56 | 1.56 | 1.56 |
| C. glabrata | 1.56 | 3.125 | 6.25 | 1.56 | 1.56 | 12.5 | 3.125 |
| C. krusei | 6.25 | 1.56 | 1.56 | 1.56 | 1.56 | 1.56 | 3.125 |
| (KCTC7273) | | | | | | | |
| C. glulliermondi | 1.56 | 3.125 | 3.125 | 0.78 | 1.56 | 3.125 | 1.56 |
| C. parapsilosis | 1.56 | 3.125 | 3.12 | 1.56 | 1.56 | 6.5 | 6.25 |

TABLE 12
MIC data of the compounds

| | Compound | Compound | Compound | Compound | Compound | | |
|---|---|---|---|---|---|---|---|
| Fungus | No. 135 | No. 148 | No. 149 | No. 150 | No. 151 | Minacozole | Amphotericin B |
| C. albicnas ATCC | 6.25 | 0.78 | 0.78 | 1.56 | 1.56 | 6.25 | 1.56 |
| 10231 | | | | | | | |
| C. albicans IFO 1385 | 6.25 | 1.56 | 3.125 | 6.25 | 6.25 | 3.125 | 1.56 |
| C. albicans ATCC | 6.25 | 1.56 | 0.78 | 1.56 | 3.125 | 3.125 | 1.56 |
| 11651 | | | | | | | |
| C. albicans ATCC | 3.125 | 1.56 | 1.56 | 1.56 | 3.125 | 0.78 | 3.125 |
| 28838 | | | | | | | |
| C. albicans OY-003 | 3.125 | 1.56 | 0.78 | 0.78 | 1.56 | 1.56 | 0.78 |
| C. albicans OY-019 | 6.25 | 1.56 | 1.56 | 1.56 | 3.125 | 1.56 | 0.4 |
| C. albicans U.K | 6.25 | 1.56 | 0.78 | 0.78 | 1.56 | >100 | 3.125 |
| C. glabrata | 12.5 | 3.125 | 6.25 | 12.5 | 3.25 | 100 | 1.56 |
| C. krusei(KCTC7273) | 1.56 | 1.56 | 1.56 | 1.56 | 1.56 | 3.125 | 0.78 |
| C. glulliermondi | 3.125 | 1.56 | 1.56 | 1.6 | 1.56 | 3.125 | 1.56 |
| C. parapsilosis | 12.5 | 3.125 | 6.25 | 12.5 | 12.5 | 3.125 | 3.125 |

Moreover, the relative activity for sterol-24-methyl transferase of the above compound in Table 11 is described in Table 13.

TABLE 13
The invitro relative activity against main compound of the above
chemical fomula (I) (+: 50 μM or more of IC50 value, ++: 5~50 μM
of IC50 value, +++: 5 μM or below of IC50 value)

| Compound No. | Relative Activity |
|---|---|
| 11 | + |
| 12 | ++ |
| 13 | +++ |
| 27 | + |
| 28 | + |
| 29 | +++ |
| 30 | +++ |
| 34 | + |
| 35 | + |
| 36 | ++ |
| 37 | ++ |
| 38 | +++ |
| 43 | + |
| 44 | +++ |
| 45 | + |
| 46 | +++ |
| 51 | + |
| 52 | + |
| 53 | ++ |
| 54 | +++ |
| 58 | + |
| 59 | +++ |
| 60 | +++ |
| 67 | + |
| 68 | ++ |
| 74 | ++ |
| 77 | ++ |
| 88 | ++ |
| 89 | +++ |
| 99 | ++ |
| 100 | +++ |
| 108 | + |
| 109 | +++ |
| 112 | + |
| 113 | +++ |
| 115 | ++ |
| 118 | ++ |
| 119 | +++ |
| 120 | +++ |
| 121 | ++ |
| 122 | ++ |
| 124 | ++ |
| 125 | ++ |
| 127 | +++ |
| 128 | +++ |
| 132 | +++ |
| 133 | +++ |
| 134 | +++ |
| 135 | ++ |
| 137 | +++ |
| 146 | ++ |
| 147 | +++ |
| 148 | +++ |
| 149 | ++ |
| 150 | +++ |
| 151 | +++ |

Meanwhile, the toxicity test of the compound in table II of the present invention was performed using mouse. The compound was suspended in propylene glycol. The resulting suspension was medicated respectively on 6 female rats and 5 male rats (SD) of which age are 5 weeks, via oral after 12 hours starvation. The general symptoms, weight change and lethal case of the above rats were investigated.

In cases of the tests of the compounds (Compound No. 12, 119, 134, 148, 150)(delivery of 1,000 mg/kg), the general symptom and weight change were normal and the lethal case was not observed. Moreover, the bacterial reverse mutation test (Ames test) using salmonella typhimurium, the chromosome aberration test using cultured lung cells derived from chinese hamster and the micronucleus test using male ICR mice on the compounds (Compound No. 12, 119, 134, 148, 150) exhibit negative results without exception.

The toxicity data for the compounds (Compound No. 12, 119, 134, 148, 150) is described in the following Table 14.

TABLE 14
The toxicity data on compounds Nos. 12, 119, 134, 148, 150

| | | | | | Genetic toxicity | | |
|---|---|---|---|---|---|---|---|
| | Acute toxicity | | | | | Chromosome | |
| Compound | | Delivery | | | AMES | abmormality | Nucleus |
| No. | Animal | route | Sex | LD50 | test | test | test |
| Compound | rats | oral | male female | >1500 | negative | negative | negative |
| No. 12 | | | | >1000 | | | |
| Compound | rats | oral | male female | >2500 | negative | negative | negative |
| No. 119 | | | | >1500 | | | |
| Compound | rats | oral | male female | >1200 | negative | negative | negative |
| No. 134 | | | | >1000 | | | |
| Compound | rats | oral | male female | >1500 | negative | negative | negative |
| No. 148 | | | | >1200 | | | |
| Compound | rats | oral | male female | >2000 | negative | negative | negative |
| No. 150 | | | | >1500 | | | |

As evident from the above descriptions, the present invention is effective in treatment of fungal infections and safe in an aspect of toxicity.

1. A 3,4-dihydroisoquinolinium salt derivative of following chemical formula (I): 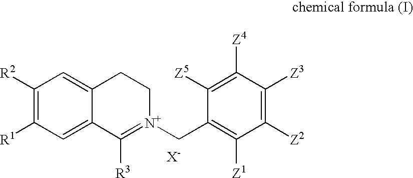
wherein
R¹ and R² which may be the same or different from each other, represent a hydrogen, halogen or alkoxy or together represent a methylenedioxy group, C₁-C₂ alkoxycarbonylamino group or C₁-C₃ alkylamino group;
R³ represents hydrogen, alkyl group, C₁-C₁₈ alkenyl, phenyl, substituted phenyl, benzyl or aryalkyl group;
Z¹, Z², Z³, Z⁴ and Z⁵ which may be the same or different, represent hydrogen, halogen, C₁-C₅ alkyl, trifluoromethyl, phenyl, substituted phenyl, nitro group, C₁-C₄ alkoxy, trifluoromethoxy, hydroxy, phenoxy, substituted benzyloxy, methoxycarboxyl group, C₁-C₄ alkoxycarbonyl or ammonium; and
X⁻ represents an inorganic acid ion, an organic acid ion or a halide.

2. The 3,4-dihydroisoquinolinium salt derivative according to claim 1, wherein R¹ and R² which may be the same or different from each other, represent C₁-C₁₀ alkoxy group.

3. The 3,4-dihydroisoquinolinium salt derivative according to claim 1, wherein R³ represents C₁-C₁₈ alkyl group.

4. The 3,4-dihydroisoquinolinium salt derivative according to claim 1, wherein R³ represents substituted arylalkyl group.

5. An antifungal compound of 3,4-dihydroisoquinolinium salt derivative of following chemical formula (I). 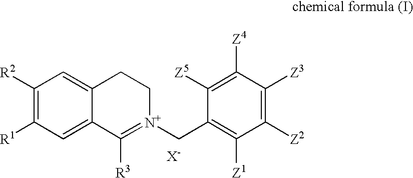
wherein, R¹, R², R³, Z¹, Z², Z³, Z⁴, Z⁵ and X⁻ are the same as defined in claim 1.

6. An isoquinolinium salt derivative of following chemical formula (II): 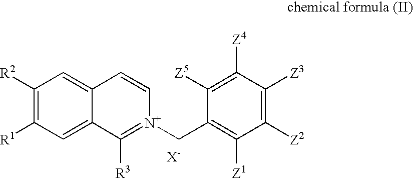
wherein
R¹ and R² which may be the same or different from each other, represent a hydrogen, halogen or alkoxy or together represent a methylenedioxy group, C₁-C₂ alkoxycarbonylamino group or C₁-C₃ alkylamino group;
R³ represents hydrogen, alkyl group, C₁-C₁₈ alkenyl, phenyl, substituted phenyl, benzyl or arylakyl group;
Z¹, Z², Z³, Z⁴ and Z⁵ which may be the same or different, represent hydrogen, halogen, C₁-C₅ alkyl, trifluoromethyl, phenyl, substituted phenyl, nitro group, C₁-C₄ alkoxy, trifluoromethoxy, hydroxy, phenoxy, substituted benzyloxy, methoxycarboxyl group, C₁-C₄ alkoxycarbonyl or ammonium; and
X⁻ represents an inorganic acid ion, an organic acid ion or a halide.

7. The isoquinolinium salt derivative according to claim 6, R¹ and R² which may be the same or different from each other, represent C₁-C₁₀ alkoxy group.

8. The isoquinolinium salt derivative according to claim 6, R³ represents C₁-C₁₈ alkyl group.

9. The isoquinolinium salt derivative according to claim 6, R³ represents substituted arylalkyl group.

10. An antifungal compound of isoquinolinium salt derivative of following chemical formula (II). 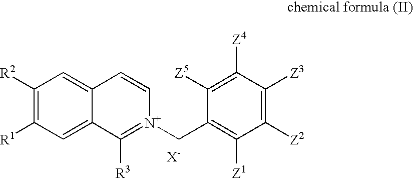
wherein, R¹, R², R³, Z¹, Z², Z³, Z⁴, Z⁵ and X⁻ are the same as defined in claim 6.

11. A pharmaceutical formulation which comprises pharmaceutically effective amount of 3,4-isoquinolinium salt derivative of following chemical formula (I). 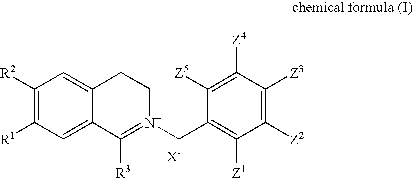
wherein, R¹, R², R³, Z¹, Z², Z³, Z⁴, Z⁵ and X⁻ are the same as defined in claim 1.

12. The pharmaceutical formulation according to claim 11, wherein the formulation has antifungal activity.

13. The pharmaceutical formulation which comprises pharmaceutically effective amount of isoquinolinium salt derivative of following chemical formula (II): 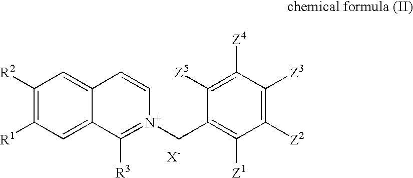
wherein, R¹, R², R³, Z¹, Z², Z³, Z⁴, Z⁵ and X⁻ are the same as defined in claim 5.

14. The pharmaceutical formulation according to claim 11, wherein the formulation has antifungal activity.

15. A process for preparing a 3,4-isoquinolinium salt derivative which comprises, □) a step for preparing a compound of following chemical formula (VI) by reacting a compound of following chemical formula (ml) with a compound of following formula (IV);
□) a step for preparing a compound of chemical formula (VII) by reacting the compound of formula (VI) obtained in the above step □) with a acyl halide; and
□) a step for reacting the compound of chemical formula (VII) obtained in the above step □) in the presence of catalyst: 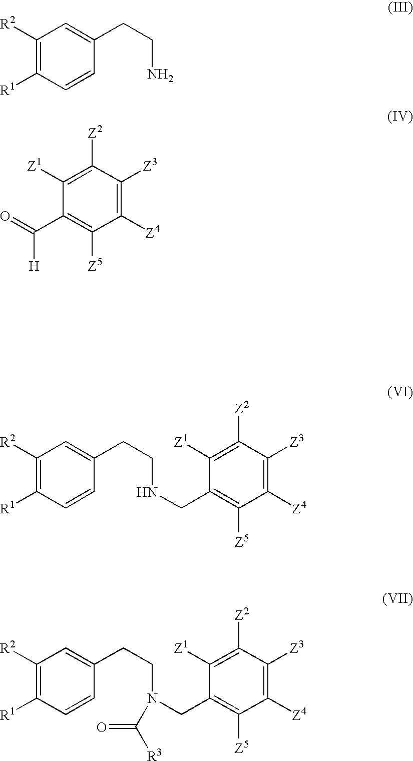
wherein, R¹, R², R³, Z¹, Z², Z³, Z⁴, Z⁵ and X⁻ are the same as defined in claim 1.

16. A process for preparing a 3,4-isoquinolinium salt derivative which comprises, □) a step for preparing a compound of following chemical formula (VI) by reacting a compound of following chemical formula (ml) with a compound of following chemical formula (V);
□) a step for preparing a compound of chemical formula (VII) by reacting the compound of chemical formula (VI) obtained in the above step □) with a acyl halide; and
□) a step for reacting the compound of chemical formula (VII) obtained in the above step □) in the presence of catalyst: 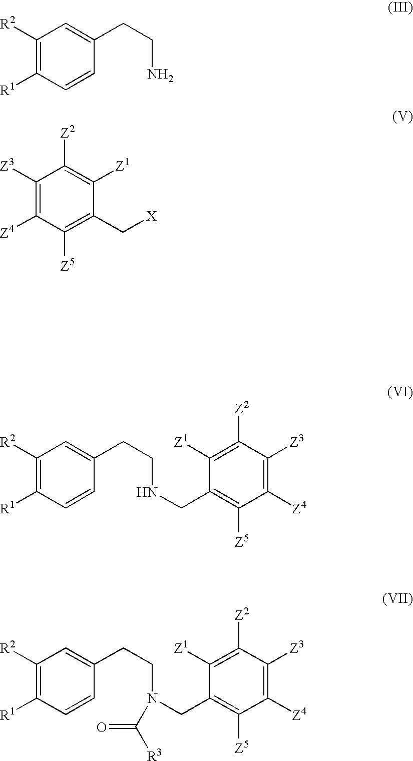
wherein, R¹, R², R³, Z¹, Z², Z³, Z⁴, Z⁵ and X⁻ are the same as defined in claim 1.

17. A process for preparing a 3,4-isoquinolinium salt derivative which comprises, □) a step for preparing a compound of following chemical formula (□) by reacting a compound of following chemical formula (III) with a acyl halide;
□) a step for preparing a compound of chemical formula (IX) by reacting the compound of chemical formula (n) obtained in the above step □) in the presence of catalyst; and
□) a step for reacting the compound of chemical formula (IX) obtained in the above step □) with a compound of following chemical formula (V): 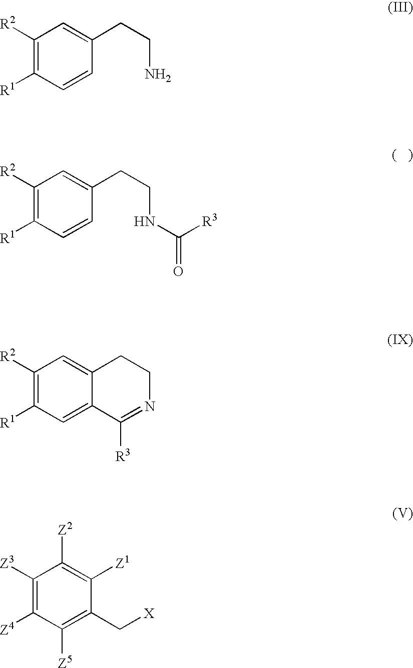
wherein, R¹, R², R³, Z¹, Z², Z³, Z⁴, Z⁵ and X⁻ are the same as defined in claim 1.

18. A process for preparing an isoquinolinium salt derivative by reacting a compound of following chemical formula (X) with a compound of following chemical formula (V): 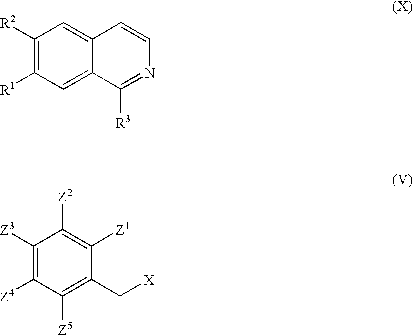
wherein, R¹, R², R³, Z¹, Z², Z³, Z⁴, Z⁵ and X⁻ are the same as defined in claim 6.
